(19) United States
(12) Patent Application Publication
(10) Pub. No.: US 2009/0076368 A1
(43) Pub. Date: Mar. 19, 2009

(54) Integrated imaging workstation and a method for improving, objectifying and documenting in vivo examinations of the uterus

(75) Inventor: Constantinos Balas, Athens (GR)

Correspondence Address: LAHIVE & COCKFIELD, LLP;FLOOR 30, SUITE 3000, ONE POST OFFICE SQUARE, BOSTON, MA, 02109, US

(73) Assignee: Forth Photonics Ltd., London (GB)

(21) Appl. No.: 12/082,672

(22) Filed: Apr. 11, 2008

Related U.S. Application Data
(60) Provisional application No. 60/923,121, filed on Apr. 11, 2007.

(51) Int. Cl. A61B 5/05

(52) U.S. Cl. 600/407

(57) ABSTRACT

Exemplary embodiments provide an imaging workstation with improved ergonomics, and a method for improving, objectifying and documenting in vivo examinations of the uterus. The imaging workstation may include an imaging head and a display, and may include mechanical supporting structures that allow the imaging head, display, and examination area to be simultaneously placed in an examiner's field of view. The imaging workstation may include means for the uniform application of a diagnostic marker. The method may involve acquiring a reference image of a tissue surface, applying a diagnostic marker and initiating image acquisition, acquiring tissue images in time sequence, aligning the captured images, and calculating dynamic optical curves and dynamic optical parameters from the images. The curves and parameters may be used to create a pseudocolor map representing different functional or structural features represented in the images, or different pathologies.

### RELATED APPLICATION

This application claims the benefit of U.S. Provisional Application No. 60/923,121, filed on Apr. 11, 2007, the contents of which are incorporated herein by reference.

### TECHNICAL FIELD

The present disclosure relates to a device for performing in vivo examinations of the uterus. In particular, an imaging workstation and a method for improving, objectifying and documenting such examinations is disclosed.

### BACKGROUND

Women with abnormal Pap-tests are often referred for colposcopic examination. The purpose of the examination is to locate abnormal areas for biopsy sampling. Colposcopy is an established procedure involving the examination of a woman's lower genital track, and in particular the area in the vicinity of the transformation zone. Colposcopic examination involves the insertion of a speculum to open the vagina to allow an examiner to observe the cervix. A colposcopy is performed with the aid of either a low magnification microscope or a camera lens with or without zoom optics.

Diagnostic chemical markers, such as acetic acid solutions, assist an examiner in localizing abnormal areas. These diagnostic chemical markers are administered topically and provoke a transient alteration of the optical properties of the tissue. The alterations appear as color alterations (the acetowhitening (AW) effect), thus enhancing contrast for the examiner. This allows the examiner to localize and identify suspicious areas for diagnosis, biopsy sampling, and treatment. The induced alterations are observed in various magnifications performed during the evolution of the AW effect. Recent clinical trials have shown that the measured in vivo dynamic optical phenomena and parameters are highly statistically correlated with the cervical neoplasia grade. The AW effect lasts 3-8 minutes depending on the neoplasia grade.

However, conventional colposcopic workstations suffer from poor ergonomics. Generally, the examiner holds the speculum in a proper position with one hand, providing the optimum field-of-view. With the other hand, the examiner manipulates the colposcope for microscopic examination, while observing through binoculars. This can be uncomfortable for the examiner.

Colposcopes equipped with a camera and display monitor improve the comfort of the examiner, but have their own ergonomic problems. Due to the space restrictions of the examination field, the monitor is normally located outside the examiner's viewing angle. In many cases the monitor may be located behind the examiner, which forces the examiner to turn around to view the monitor. Another drawback of existing digital and video colposcopes is that they may not provide stereo imaging, which is essential for performing treatment and biopsy and for observing surface elevation effects associated with the AW phenomenon.

Further, both optical and digital colposcopes may not enable inspection of the endocervical canal. This is a significant clinical limitation, because many neoplasias are developed in the vicinity of the transformation zone of the endocervical canal.

Another drawback of existing colposcopes involves their optical zooming facilities, which are used to magnify suspicious sub areas of the examined tissue. Optical zooming may cause a loss of examined area overview. That is, the viewable area may be reduced when optical zooming is used. As a result, the AW responsive area may be located outside the zooming window, and therefore may remain undetected. Zooming in and out cannot address this limitation since AW evolution is relatively fast. This limitation of existing colposcopes is directly associated with the high risk for abnormal areas to remain undetected and to progress to invasiveness and metastases.

Further, conventional colposcopes do not provide a control on the quantity and application uniformity of the diagnostic chemical marker. In order to maintain the AW effect for longer times, the examiner repeatedly applies the marker. The lack of control over quantity and uniformity substantially affects the AW effect, which may result in over-diagnosis and unnecessary biopsies. In addition, multiple applications of the marker results in the excess accumulation of the marker, which may obstruct the area under examination.

Further, conventional colposcopes may not provide quantitative diagnostic information. Rather, diagnostic performance relies totally on the experience and visual acuity of the examiner. Due to the dynamic nature of the AW effect and to the visual limitations of the human optical system in memorizing dynamic phenomena, colposcopy is subject to a high biopsy sampling error rate. Various studies have reported high levels of inter- and intra-observer disagreement, while average diagnostic performance is very low. Conventional colposcopes may not provide guidance for biopsy sampling, nor recording and documentation of the biopsy sampling procedure. Such documentation is useful in order to elucidate whether a negative histological assessment refers to a healthy tissue sample or to a sampling error.

Due to the subjective nature of the procedure, colposcopy does not provide a definitive diagnosis. Instead, examiners use colposcopy to locate abnormal areas for biopsy sampling. The obtained biopsy samples are then submitted for histological examination, which provides the diagnosis. Colposcopy's sensitivity is reported to range from 56-67% and its specificity from 54-80%. It is a subjective process, dependent on the skill and experience of the operator.

The diagnostic deficiencies of colposcopy are largely attributed to two factors. First, the degree of correlation between observable macroscopic tissue features and actual tissue pathology is not well known. Second, there are few quantitative methods for assessing these tissue features in vivo.

Recent clinical trials have shown that the measurement and mapping of dynamic optical phenomena provoked by the topical application of diagnostic markers could provide a means for improving, objectifying and documenting colposcopy.

### BRIEF SUMMARY

Exemplary embodiments provide an imaging workstation with improved ergonomics, and a method for improving, objectifying and documenting in vivo examinations of the uterus. The imaging workstation may include an imaging head and a display, and may further include mechanical supporting structures that allow the imaging head, display, and examination area to be simultaneously placed in an examiner's field of view. The imaging workstation may further include means for the uniform application of a diagnostic marker. The method may involve acquiring a reference image of a tissue surface, applying a diagnostic marker and initiating image acquisition, acquiring tissue images in time sequence, aligning the captured images, and calculating dynamic optical curves and dynamic optical parameters from the images. These dynamic optical curves and dynamic optical parameters may be used to create a pseudocolor map representing different functional or structural features represented in the images or different pathologies.

According to exemplary embodiments, an imaging workstation with improved ergonomics is disclosed. According to one embodiment, the imaging workstation allows an examiner to inspect a digital image on electronic display means. With the aid of mechanical supporting structures, the electronic display means, an examination area, an imaging sensor and imaging optics may be simultaneously located within the examiner's viewing angle. The imaging workstation may be portable.

According to another embodiment, both stereo digital and endoscopy are integrated in one imaging workstation. This allows the imaging workstation to image the cervix and the endocervical canal of the uterus through a dual sensor stereo display integrated with an endoscope.

According to another embodiment, the imaging workstation mechanically stabilizes the speculum in relation to the imaging sensor. The imaging workstation may use lockable supporting structures in conjunction with an imaging head unit and a speculum. This allows the examiner to apply the diagnostic marker uniformly. Further, the examiner can maintain substantially the same field-of-view while monitoring dynamic optical phenomena of diagnostic importance.

According to another embodiment, the imaging workstation includes an imaging unit. The imaging unit provides a shadow-free, high-quality overview image, image-enhancing optics and executable software improving image quality. The imaging unit also allows for local magnification by providing a properly designed imaging unit image, display size, and resolution.

According to another embodiment, the imaging workstation includes mechanical structures, such as a base member, a planar positioning structure, a space micro-positioning structure, and a pivoting structure. The base member provides a stable platform for these structures. The planar positioning structure allows critical components to be manually translated in close proximity to the examination area. The space micro-positioning and pivoting structures allow the examiner to make micromanipulations useful for mechanically connecting an optical imaging module with a speculum. After establishing the connection, motion-locking mechanisms can be activated to insure stable imaging conditions for the duration of the examination.

According to another embodiment, the imaging workstation standardizes marker application uniformity and quantity. The marker application may also be synchronized with the image capturing procedure. These objects may be achieved with proper marker applicators, sensors and control electronics mounted on lockable supporting structures.

According to another embodiment, the imaging workstation increases the objectivity of colposcopic diagnosis. This is achieved by reliably and quantitatively assessing dynamic optical characteristics of the tissue (i.e., optical characteristics of tissue that change over time). These optical characteristics may be provoked by topically applying diagnostic markers. Reliable measurements are achieved with proper mechanical stabilization and marker application standardization, combined with digital image and signal processing. This digital signal processing eliminates artifacts and calculates, and maps, dynamic optical parameters with high diagnostic value.

According to another embodiment, the workstation automatically detects abnormal areas. The workstation may automatically detect lesion quantitative information, such as the lesion's size distribution as a function of the grade. This may be achieved by automatically segmenting a dynamic map.

According to another embodiment, the workstation guides biopsy sampling and treatment by automatically detecting abnormal areas and super-positioning digital markings onto a real time displayed image. This enables dynamic-map-guided surgical treatment, laser treatment and biopsy sampling.

According to another embodiment, the workstation provides biopsy sampling and treatment procedures, together with dynamic imaging data, a patient's personal data, past examinations, and diagnostic tests. This may enable a review of the examination. It may also facilitate off-site digital window-based microscopy, telemedicine and comparison with subsequent examinations for objective follow-up.

According to one embodiment, a supporting structure for an integrated portable imaging workstation operable by an examiner for improving, objectifying and documenting in vivo examination of the uterus is provided. The workstation may include at least an imaging head module operably-connected to the supporting structure, for imaging an examination area of a patient situated on an examination platform, wherein the supporting structure controls movement and positioning of at least the imaging head module in to an imaging position in close proximity to said examination area and away from said examination area allowing for the patient's access to the examination area and comprises control means for locking the imaging head module in position in the examination area and unlocking to allow translation away from the examination area.

According to one embodiment, a supporting structure for an integrated portable imaging workstation operable by an examiner for improving, objectifying and documenting in vivo examination of the uterus is provided. The workstation may include at least an imaging head module operably-connected to the supporting structure, for imaging an examination area of a patient situated on an examination platform. The supporting structure may include: (a) a base member; (b) a planar positioning structure mounted onto the said base member in a manner such that said planar positioning structure can move, relative to the base member, from a position away from the examination area, allowing for the patient's access to the examination platform, to an imaging position, translating at least said imaging head module in close proximity with the examination area; (c) a space micro-positioning structure disposed directly onto the said planar positioning structure; (d) a weight counterbalancing mechanism integrated in said space micro-positioning structure; (e) a pivoting structure disposed directly onto said space micro-positioning structure, wherein the imaging head module is disposed directly on the pivoting structure, wherein motion of the space micro-positioning structure and the pivoting structure may be locked to fix the imaging head module in position in the examination area and unlocked to allow translation away from the examination area; and (g) a handle for the control of the position of said space micro-positioning and pivoting structures.

According to one embodiment, an integrated portable imaging workstation for improving, objectifying and documenting in vivo examinations of the uterus is provided. The integrated portable imaging workstation may include a supporting structure, which may include one or more of: a base member including an eccentric ellipsoid shape, further comprising rotational members with an allowable range of motion of about 90°; a planar positioning structure including an articulating extension mounted onto the rotating members of the base member and wherein the planar positioning structure is a relatively longish member with a vertically supporting foot, fixed near to its other end, with a lockable, integrated wheel, and wherein following the range of motion allowed by the rotating members, the planar positioning structure rotates from its extended (rest) position, allowing for the patient's access to the examination platform, to its closed (imaging) position, translating at least the imaging head module in close proximity with the examination area; a space micro-positioning structure including an XYZ translator disposed directly onto the planar positioning structure; a weight counterbalancing mechanism integrated in the space micro-positioning structure, wherein the suspended weight is balanced using constant force springs mounted fixedly to the Z-axis motion element; a pivoting structure disposed directly on the space micro-positioning structure wherein the pivoting structure comprises a limited ball joint, and wherein XY motion of the XYZ translator is locked/unlocked using electromagnetic means, Z motion of the XYZ translator is locked/unlocked using a motor coupled with a timing belt and pulley, and the pivoting structure motion is locked/unlocked using counteracting compression springs and a cam-follower mechanism; and a handle for the control of the position of the space micro-positioning and pivoting structures is disposed onto the pivoting structure, further incorporating a microswitch to trigger substantially the locking/unlocking of the XY, Z and ball joint motions.

The integrated portable imaging workstation may also include an imaging head module disposed directly onto the pivoting structure. The imaging head module may include: an imaging sensor including at least one CCD sensor, coupled with a polarizer with a first orientation of its polarization plane; an imaging lens including a lens with at least 20 mm focal length; a light source means including a white LED light source equipped with optical elements for light beam focusing on an examination area and wherein the light source is coupled with a polarizer with a second orientation of its polarization plane and wherein the second orientation is adjusted to become substantially perpendicular with the first polarization plane, where at least one of the imaging sensor and the illumination means are affixed on the second mechanical support and wherein the second mechanical support is affixed on the pivoting structure through a linear slider for fine focusing; beam manipulation optics including at least one light deflector for deflecting the light rays of at least one of the imaging and illumination means to become substantially co-axial and wherein the light deflector is placed distantly enough from the one of the imaging and illumination means, that is subjected light ray deflection, forming a clear aperture from which the light rays of the other of the imaging and illumination means pass substantially unobstructed; a diagnostic marker dispenser including a bottle containing a volume of the diagnostic marker and is connected via a 2-way valve and tubing to a syringe-like mechanism of fixed volume, and a narrow angle, full-cone, axial spray nozzle, and wherein the nozzle is detachably connected with the extension bracket and aligned properly so that the marker is uniformly applied onto an examination area covering at least the imaging sensor's field-of-view and wherein the nozzle is connected with the syringe-like mechanism via tubes and the valves for transferring to and dispensing from the nozzle the marker, and wherein the syringe-like mechanism is housed in an appropriately designed casing comprising one or more photosensors for detecting the complete depression of the syringe-like mechanism and wherein the output signal of the photosensors is used to synchronize image capturing with application of the diagnostic marker; a speculum shaft detachably connectable with the first mechanical support via mechanical locking means disposed onto the first mechanical support via an extension bracket and wherein the locking means is a bayonet type mechanism and wherein the bayonet type mechanism comprises a pre-loaded sleeve with an incorporated angled groove, and a pre-load mechanism for the sleeve, by means of which an extension shaft at the back side of the vaginal speculum is locked into the sleeve, and wherein the pre-loaded sleeve comprises a receptacle for the extension shaft attached to the speculum shaft and wherein the speculum shaft has a dowel pin pressed through it close to its distal end and perpendicular to the axis of the speculum shaft and wherein the dowel pin mates with the receptacle, and wherein the speculum extension shaft comprises shape features to spatially position the speculum longitudinal axis substantially coaxially with the central imaging and illumination axes inside the speculum, when the speculum shaft is locked on the first mechanical support; computer means disposed directly onto the XY member of the space micro-positioning structure, wherein the computer means is based on multiple core microprocessor which different cores handling different tasks in parallel, and wherein the computer means further includes control means for controlling at least the locking mechanisms and for synchronization and triggering image capturing with agent application, computer memory means, and hardware interface means for connecting computer peripherals including but not limited to: one or more displays, user interface means, a local network, hospital data bases, the internet, and/or printers; user interface means, wherein the user interface means are selected from among touch-screen, a keyboard, a wireless keyboard, a voice interface, a foot-switch or combinations thereof; display means, wherein the display means are selected from among, monitors, a touch-screen monitors, head-mounted displays, video goggles and combinations thereof, and wherein the monitor is placed on one side of an examination platform and is disposed directly onto the base member and wherein the monitor is positioned spatially so as to be within the viewing angle (or field of vision) of the user and wherein the viewing angle (or field of vision) also includes the examined area and the imaging head module; and software means wherein the software is used for programming the computer to perform at least in part one or more of the following functions: image calibration; image capturing initialization; image registration; dynamic curve calculation; processing and analysis; dynamic pseudocolor map calculation and segmentation; biopsy sampling/treatment guiding documentation; image magnification; and/or data base operations for storing, retrieval and post-processing images and data.

According to one embodiment, an integrated portable imaging workstation for improving, objectifying and documenting in vivo examinations of the uterus is provided. The integrated portable imaging workstation may include a diagnostic marker dispenser. The integrated portable imaging workstation may include an imaging head module for imaging an examination area including one or more of: an imaging sensor, imaging optics and/or a light source; a means for generating a triggering signal for activating image capturing in a synchronized manner with the application of the diagnostic marker; computer means connected at least to the imaging head module; display means connected to the computer means for displaying an image of said examination area; user interface means; and a computer readable medium holding computer program instructions. The computer readable medium may hold computer program instructions, causing the workstation to do one or more of the following actions: store a reference image in the computer memory means of the computer; capture and store a new reference image replacing the previously stored reference image in the computer memory means; repeat this procedure until receiving a triggering signal and use the signal for triggering and synchronization of initiation of the image capturing procedure, generated with completion of the application of the diagnostic marker; store the most recently captured image, just before the arrival of the triggering signal, to be used as reference image; and initiate the capture, store and display images in time sequence; and at predetermined time intervals and duration. The computer readable medium may hold computer program instructions, causing the workstation to do one or more of the following actions: align the reference image and the images captured in time-sequence; calculate and display the remitted light intensity versus time curves smooth said defuse reflectance vs. time curves using algorithms selected from a group comprising: Butterworth, Fast Fourier Transformation, single and multiple exponential fitting based filters, difference based filters or combinations thereof; calculate from the original or fitted/smoothed curves a group of dynamic optical parameters including: time integral, defined as the area under a curve of the remitted light intensity versus time curve calculated for at least in part of the predetermined time duration of the acquisition process; maximum; time-to-max the curve slopes or combinations thereof; assign pseoudocolors to the parameter value ranges, to generate the dynamic pseudocolor map representing the spatial distribution of the parameter ranges; display and overlay the map onto the tissue image; and align the map with at least the reference image for highlighting abnormal areas and for documenting dynamic optical effects through a single image.

According to one embodiment, an integrated portable imaging workstation for improving, objectifying and documenting in vivo examinations of the uterus is provided. The integrated portable imaging workstation may include: an imaging head module for imaging an examination area, comprising one or more of an imaging sensor, imaging optics and/or a light source; computer means connected to the imaging head module; display means connected to the computer means for displaying an image of said examination area; user interface means, and; software means installed in the computer means, which cause the computer means to capture, store and process images obtained by the imaging head module to permit display of an image of the examination area by the display means. The imaging sensor may have a first spatial resolution, the imaging optics may be a lens providing a constant first magnification, the display means may have a given size and a second spatial resolution and the entire image captured by the sensor may be displayed at lesser than or equal to the first resolution on the display means providing a first magnification, and a second magnification may be achieved by displaying and overlaying selected image sub-areas at a resolution at least equal with the first resolution, for allowing magnification of multiple sub-areas, without moving the imaging head and without changing magnification optics, and for post examination magnification and analysis of the captured images, while maintaining the image overview.

According to one embodiment, an in vivo examination workstation for in vivo examination of a uterus is provided. The workstation may include: a diagnostic marker dispenser for dispensing a diagnostic marker; an imaging head module for acquiring an image of an examination area within the uterus, the module comprising, an imaging sensor, imaging optics, and a light source; a triggering means for generating a triggering signal for activating image acquisition of the examination area in synchronization with dispensing of said diagnostic marker from said diagnostic marker dispenser; a computer means connected at least to the imaging head module, the computer means programmed to cause said workstation to, acquire a reference image, store the reference image, in response to the triggering signal, synchronizing the dispensing of the diagnostic marker and the acquiring of a plurality of images of the examination area, store and display the acquired images in time sequential manner at predetermined time intervals and duration, align said reference image and the images captured in time-sequence, calculate and display one or more remitted light intensity vs. time curves, smooth said remitted light intensity vs. time curves using an algorithm selected from a group consisting of: Butterworth, Fast Fourier Transformation, single and multiple exponential fitting based filters, difference based filters or combinations thereof, calculate from said original or smoothed curves a group of dynamic optical parameters having one or more values, the group including: time integral, defined as area under the curve of said remitted light intensity vs. time curve calculated for at least in part of said predetermined time duration of the acquisition process, maximum, time-to-max curve slopes, or combinations thereof, assign pseoudocolors to multiple ranges of said parameter values, to generate a dynamic pseudocolor map representing a spatial distribution of said parameter ranges, display and overlay said dynamic pseudocolor map onto one of the acquired images, and align said map with at least said reference image for highlighting abnormal areas and for documenting dynamic optical effects through a single image of the tissue, a display means connected to the computer means for displaying an image of the examination area; and a user interface.

### BRIEF DESCRIPTION OF THE FIGURES

The following figures provide a better understanding of the present disclosure, when considered in conjunction with the detailed description. The following figures depict illustrative embodiments of the disclosed imaging workstation.

FIG. 1 depicts an exemplary imaging workstation for colposcopic examination.

FIG. 2 depicts an exemplary imaging head module that may be employed in the imaging workstation.

FIG. 3A depicts the imaging head module, where the imaging lens means is a microlens positioned parallel to the illumination source.

FIG. 3B depicts the imaging head module with two imaging sensors placed in close proximity to each other and coupled with the microlens of FIG. 3A

FIG. 4 depicts the imaging means and the illumination means placed at substantially right angles to each other within the imaging head module.

FIG. 5 depicts an overview of an exemplary imaging workstation, including manual means for delivering the diagnostic marker to the application probe.

FIG. 6 depicts an exemplary space micro-positioning structure suitable for use in the imaging workstation disclosed herein.

FIG. 7 depicts a way to temporarily fix the space micro-positioning structure along its axes of motion.

FIG. 8 depicts an exemplary pivoting structure suitable for practicing the illustrative embodiments taught herein.

FIG. 9 depicts exemplary imaging head modules suitable for practicing the illustrative embodiments taught herein.

FIG. 10 depicts an exemplary base member suitable for practicing the illustrative embodiments taught herein.

FIG. 11 depicts some of the capabilities of an exemplary imaging workstation according to the teachings disclosed herein

FIG. 12 depicts an algorithm for overlaying circular markings onto a real-time displayed image of an examined area.

FIG. 13 depicts a displayed image of the examined area.

FIG. 14 depicts a flow chart illustrating the basic steps in a method suitable for practicing the teachings discussed herein.

FIG. 15 depicts DOC curves obtained from cervical tissue sites interacting with acetic acid solution (the diagnostic marker) corresponding to HPV

FIG. 16 depicts DOC curves obtained from cervical tissue sites interacting with acetic acid solution (the diagnostic marker) corresponding to Inflammation

FIG. 17 depicts DOC curves obtained from cervical tissue sites interacting with acetic acid solution (the diagnostic marker) corresponding to CIN1

FIG. 18 depicts DOC curves obtained from cervical tissue sites interacting with acetic acid solution (the diagnostic marker) corresponding to High Grade Lesions

FIG. 19 depicts four parameters on the curve of a DOC: ‘Max’, ‘Tmax’, ‘SlopeA’, and ‘SlopeB.’

FIG. 20 depicts the LG/HG ROC analysis of the cumulative results for the ‘Integral’ parameter.

FIG. 21 depicts the sensitivity and specificity plots derived from the ROC analysis for various values of the ‘Integral’ parameter used for the quantification of the acetowhitening characteristics.

FIGS. 22-26 depict the mean values, with corresponding error-bars representing 95% confidence intervals, for parameters for the LG and HG diagnostic conditions, as concluded through biopsy examination performed by the histologists.

FIG. 27 depicts DOP values correlated with quantitative data expressing nuclear density obtained through quantitative pathology methods, with structural phenomena for the case of cervical cancer where acetic-acid solution is used as a biomarker.

FIG. 28 depicts DOP values correlated with quantitative data expressing nuclear density obtained through quantitative pathology methods, with structural phenomena for the case of cervical cancer where acetic-acid solution is used as a biomarker.

FIG. 29 depicts the DOC values obtained from a normal tissue site.

FIG. 30 depicts an overview of aspects of the imaging workstation and methods described herein.

### DESCRIPTION

As used herein, the term “dynamic optical curve” or “DOC” includes a curve representing an optical characteristic of tissue under observation, such as intensity of backscattered light from a tissue or portion thereof, reflectance of light, diffusive reflectance of light from a tissue or a portion thereof, or fluorescence from a tissue or a portion thereof that has been exposed to a diagnostic marker over time.

As used herein, the term “diagnostic marker” includes any chemical agent capable of altering an optical signal from the tissue sample being tested. Non-limiting examples of such agents include, but are not limited to acetic acid, formic acid, propionic acid, butyric acid, Lugol's iodine, Shiller's iodine, methylene blue, toluidine blue, osmotic agents, ionic agents, and indigo carmine. Any solutions of the foregoing agents may be used. In a preferred embodiment, the diagnostic marker is an acetic acid solution, e.g., a 3-5% acetic acid solution.

As used herein, the term “dynamic optical parameter” includes one or more parameters based on which one of ordinary skill in the art may characterize, e.g., grade, a tissue. As described herein such parameters may be derived via a mathematical analysis of one or more of the dynamic optical curves plotted based on the intensity of backscattered light from a cancer tissue, or portion thereof, that has been exposed to a diagnostic marker over time. Such parameters may also be derived by an empirical, manual, or visual analysis of one or more of the dynamic optical curves. Non-limiting examples of the dynamic optical parameters contemplated by the present invention are ‘Integral’, ‘Max’, ‘Time to Max’, ‘Area to Max’, ‘SlopeA’, and ‘SlopeB’.

The articles “a” and “an” are used herein to refer to one or to more than one (i.e. to at least one) of the grammatical object of the article. That is, the singular includes the plural. Further, the plural form of a word includes the singular form of that word. By way of example, “a dynamic optical parameter” means one or more dynamic optical parameters, while “dynamic optical parameters” may mean a single dynamic optical parameter.

As used herein, the term “tissue” includes any tissue, or portions thereof, including cancerous and pre-cancerous tissues. For example, the tissue may be an epithelial tissue, a connective tissue, a muscular tissue or a nervous tissue. The tissue may be an epithelial tissue, or a portion thereof, e.g., covering and lining epithelium or glandular epithelium. For example, the tissue may be cervical tissue; skin tissue; gastrointestinal tract tissue, e.g., oral cavity tissue, stomach tissue, esophageal tissue, duodenal tissue, small intestine tissue, large intestine tissue, pancreatic tissue, liver tissue, gallbladder tissue or colon tissue; or nasal cavity tissue. The tissue is may be a pre-cancer or cancer tissue, such as, for example, a dysplasia, a neoplasia or a cancerous lesion.

As used herein, the phrase “characterizing” a cancer tissue includes the characterization of a cancer tissue using the methods described herein such that the screening, clinical diagnosis, guided biopsy sampling and/or treatment of a cancer tissue is facilitated. For example, a cancer tissue may be graded, e.g., characterized as a low grade (LG) lesion (i.e., an HPV infection, an inflammation or a CIN Grade I lesion, or a combination thereof) or a high grade (HG) lesion (e.g. a CIN Grade II lesion, a CIN Grade III lesion, or Invasive Carcinoma (CA) or a combination thereof).

There are various degrees of cervical intraepithelial neoplasia (CIN), formerly called dysplasia. Histologically evaluated lesions are typically characterized using the CIN nomenclature; cytologic smears are typically classified according to the Bethesda system; and cervical cancer is typically staged based on the International Federation of Gynecology and Obstetrics (FIGO) system. CIN Grade I (mild dysplasia) is defined as the disordered growth of the lower third of the epithelial lining; CIN Grade II (moderate dysplasia) is defined as the abnormal maturation of two-thirds of the lining; CIN Grade III (severe dysplasia): encompasses more than two thirds of the epithelial thickness with carcinoma in situ (CIS) representing full-thickness dysmaturity. There are well known classification systems for the characterization of cervical dysplasia, i.e., the disordered growth and development of the epithelial lining of the cervix (see, for example, DeCherney, A. et al., Current Obstetric & Gynecologic Diagnosis & Treatment, 9^{th} ed., The McGraw-Hill Companies, New York, N.Y. (2003), the contents of which are incorporated herein by reference).

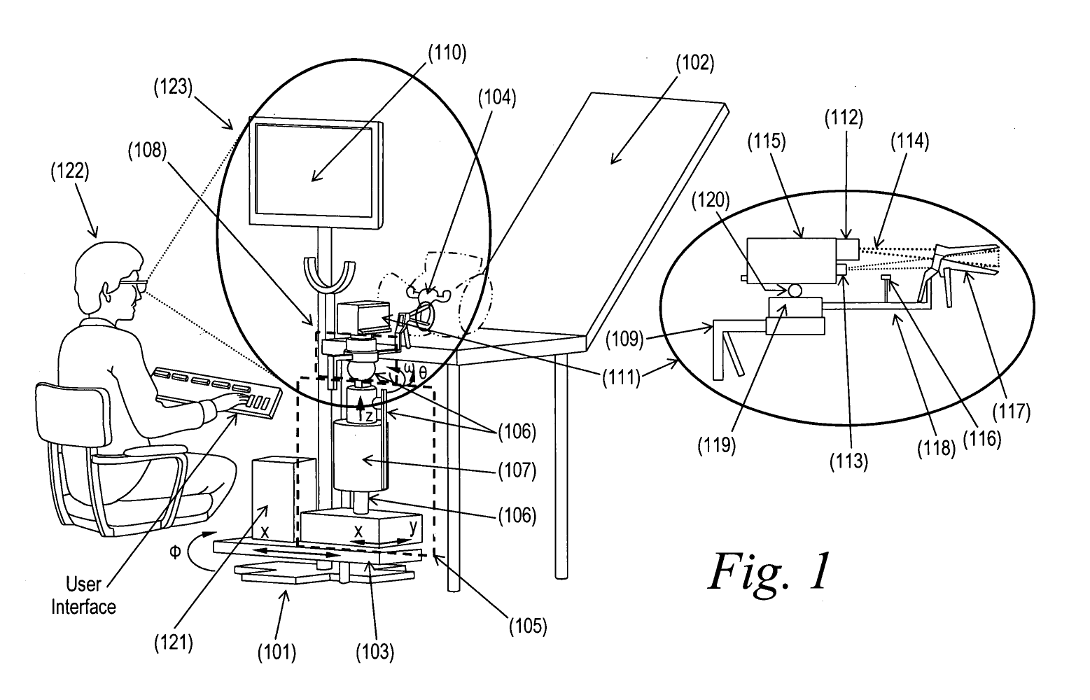

FIG. 1 depicts an exemplary imaging workstation for colposcopic examination. The exemplary imaging workstation allows an examiner 122 to inspect a digital image on electronic display means 110. The electronic display means 110, an examination area 104, an imaging sensor 115 and imaging optics 112 may be simultaneously located within the examiner's viewing angle 123. This can be achieved with the aid of mechanical supporting structures as taught herein.

A supporting structure can include a base member 101 that provides a stable platform for the workstation. The base member 101 further acts as a chassis for the mounting and coupling of components of the imaging workstation. The base member 101 can be a means of mounting the workstation on a solid datum such as a floor or a permanent fixture in the environment such as an examination platform 102 (e.g., a gynecological bed). Alternatively, the base member 101 can be an independent base member capable of being temporarily or permanently affixed to the abovementioned fixtures. Thus, the imaging workstation may be portable.

The supporting structure can further include a planar positioning structure 103, which may provide mechanical support and allow some of the components of the imaging workstation to be brought in proximity with the examination area 104. The planar positioning structure 103 may be an articulating arm with one or more articulation joints capable of positioning the arm in a two-dimensional space. The planar positioning structure 103 may be moved linearly using slides (e.g., along the x axis as shown in FIG. 1) or rotationally using articulation joints (e.g., rotated at an angle Φ as shown in FIG. 1). The articulation joints may disposed on the base member 101. The range of motion of the planar positioning structure 103 may be limited to a pre-specified range of motion. The planar positioning structure 103 may provide coarse positioning for some of the components of the workstation, such as an imaging head module 111, with respect the examination area 104. The examination area 104, imaging head module 111 and display 110 may be located substantially within the user's field-of-view.

The supporting structure may further include a space micro-positioning structure 105, which may be affixed to the planar positioning structure 103. The space micro-positioning structure 105 accurately positions components of the imaging workstation in relation to the target area to be examined. The space micro-positioning structure 105 may work in the Cartesian (x,y,z), Polar or Spherical space, or combinations thereof, to position the components of the imaging workstation. These components may include sensors and light sources, which are mounted on the space micro-positioning structure 105. The micro-positioning structure 105 may employ, for example, counteracting compression springs, rotational springs, self compensating gas dampers, hydraulic suspension elements or pneumatic means, or a combination thereof.

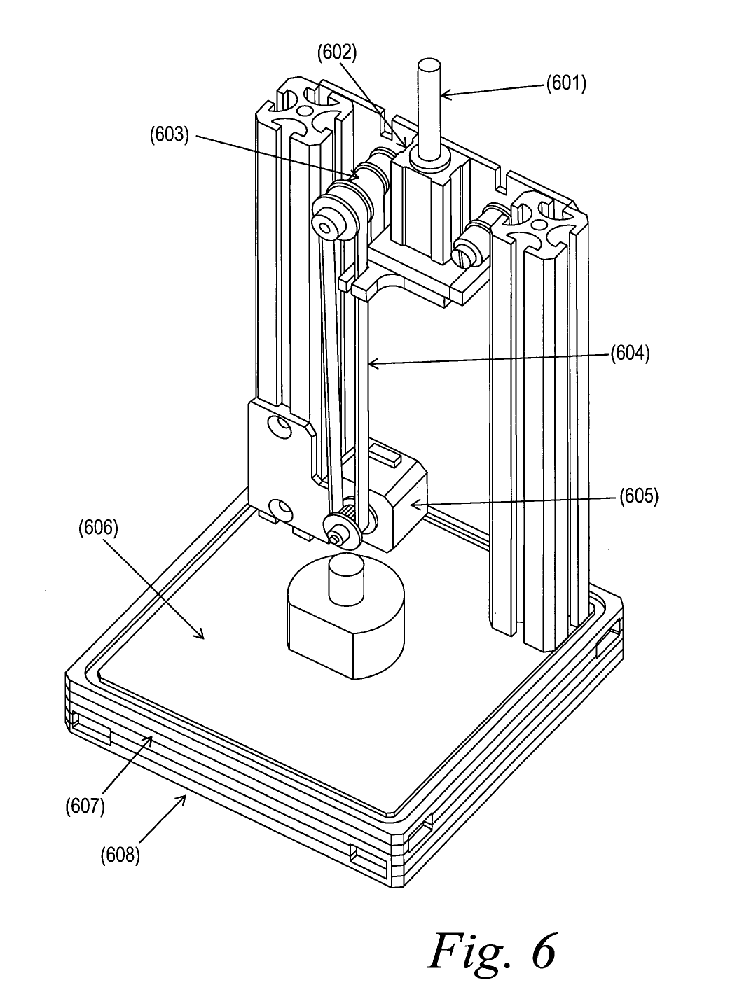

The space micro-positioning structure 105 may include a mechanism to balance the weight and the torque exerted on it. A weight counterbalance 107 assists the user in performing micromanipulations for connecting or disconnecting the imaging head module 111 with a speculum extension shaft 1010. The weight counterbalance 107 may include constant force springs 603 (FIG. 6), constant torque spring sets, counteracting compression springs, self compensating gas dampers, multi-chamber hydraulic dampers, or active pneumatic circuits and circulating and suspended pulley weights in the configuration of an Atwood's machine.

Locking mechanisms 106, for locking and unlocking the positioning structures, may allow all or some of the degrees of freedom of the planar positioning structure 103 and space micro-positioning structure 105 to be temporarily locked once the desired position has been achieved. In some embodiments, temporary locks may be activated or released by a single user action.

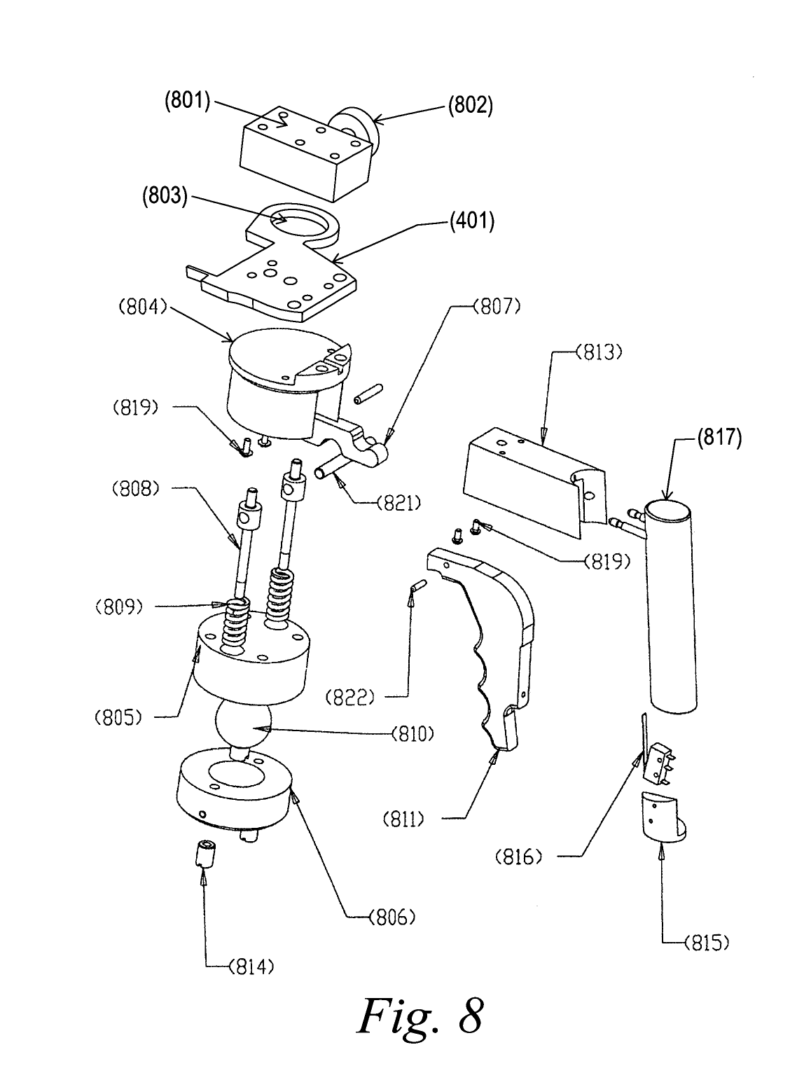

The locking may be effected by, for example, mechanical, electrical, electro-mechanical, electromagnetic, pneumatic, or hydraulic means, or electrical drive means of activating and deactivating friction inducing elements, or a combination thereof. The mechanical means may include a cam 807 (FIG. 8), mechanical stops, a high tension steel cable actuated lever, or multi-pivoting mechanisms. The electrical means may include servomotors supplied with holding torque inducing current or current to induce or change polarities in ferro-magnetic elements. The pneumatic means may include pneumatically actuated clutches to engage and disengage relatively mobile members or pneumatically actuated friction elements.

Furthermore, the positioning structures of the exemplary imaging workstation may have a number of moving parts. In some embodiments, the claimed workstation may include triggering means for controlling the friction level of one or more moving parts amongst the planar positioning structure 103, the space micro-positioning structure 105, or the pivoting structure 108. The triggering means may include manually actuated screws or knobs. Alternatively, the triggering means may be actuated with a remotely-activated mechanism, such as an actuation signal located on the handle 109. The triggering means may be analogous to the mechanism used for activating and deactivating the friction elements, and may include the use of a high leverage ratio pivoted lever, a microswitch, or a pneumatic pilot line to activate and deactivate respective pneumatic components. The handle 109 may be located directly on the pivoting structure 108, or anywhere that allows the handle 109 to be used to position the various elements.

In some embodiments, the triggering means can be a high leverage ratio pivoted hand lever 811 (FIG. 8) that compresses and decompresses suitable springs to activate and deactivate a direct manual brake for the pivoting structure 108. The hand lever 811 also acts as a means of remotely triggering the brakes of mobile members. The hand lever 811 may use remote activation and deactivation means including, mechanical, electrical, hydraulic, or pneumatic means.

In some embodiments, a manual force may be supplied to the triggering handle 109. The force may be transmitted from the triggering handle 109 to remotely located brakes using a high tension steel cable housed in an appropriately sized external sheath. The sheath may be substantially flexible but incompressible. The sheath may include an outer covering made of hardened polymeric compounds, and the inner portion of the sheath may include a continuous compression spring.

The imaging workstation's supporting structure may include a pivoting structure 108 capable of providing tilting, pitching and yawing motions (e.g., θ, ω in FIG. 1) for the components attached to it. Additionally, the pivoting structure 108 may include a temporary locking mechanism to allow the user to lock the motion of the pivoting structure 108 in one or more degrees of freedom with a single user action. This allows the user to fix the position of the components attached to the pivoting structure 108 when the desired position has been achieved. The user action may be the same user action required to activate or release the locks on the space micro-positioning structure 105, thereby allowing the locks on both the space micro-positioning structure 105 and the pivoting structure 108 to be activated or released with a single user action. The locks incorporated into the pivoting structure 108 may be, for example, mechanical, electromechanical, hydraulic, pneumatic, or a combination thereof. Additionally, a handle 109 may be provided for manually manipulating the positioning structures. The single user action may be performed using the handle 109.

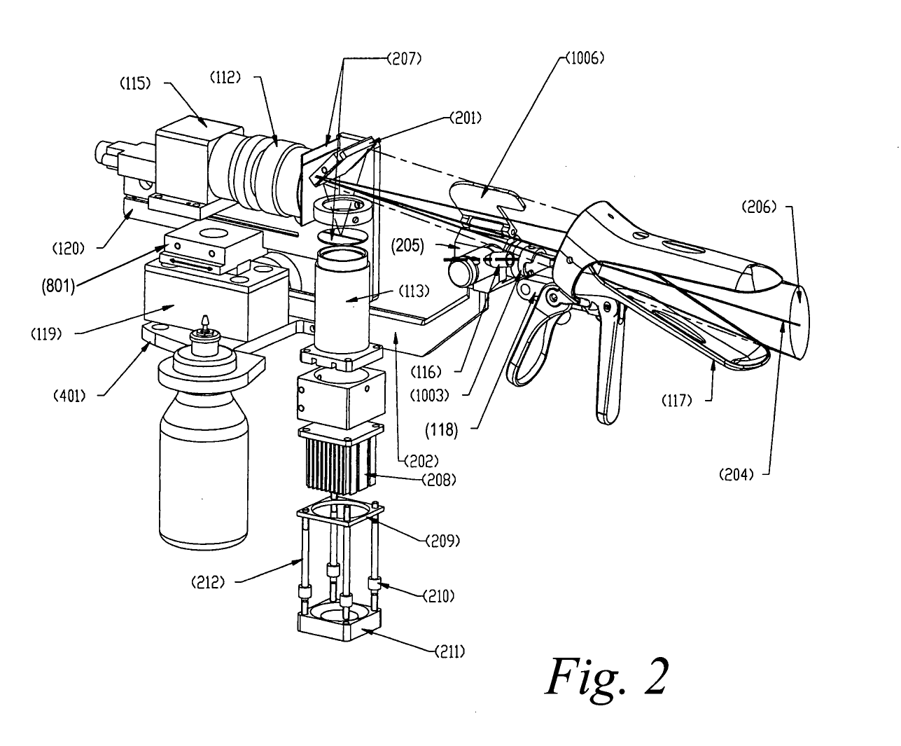

Further, one or more of the planar positioning structure 103, the space micro-positioning structure 105 and the pivoting structure 108 may have at least two translation modes. A first translation mode is a free moving mode, allowing for the manual free and counterbalanced spatial movement of the imaging head module 111 in and out of the examination area 104 before the connection and after the disconnection of the imaging module with the speculum extension shaft 1010. A second translation mode is a substantially locked mode; when the connection is established, the imaging, illumination ray symmetry axes and the agent dispensing pattern longitudinal axis become substantially collinear with the speculum's longitudinal axis 204 (seen in FIG. 2). This is achieved through proper focusing and mounting of the corresponding components at proper positions on the first and second mechanical supports, so that the imaging field-of-view, illumination from a light source 113 and the tissue area covered by the agent are substantially overlapping.

Additionally, the supporting structure may also include a display 110 for displaying images and data captured by an imaging head module 111. In one embodiment, the display's supporting structures are disposed either on the base member 101 or on the other positioning structures, so that the display 110 is within the viewing angle 123 of the user 122. The viewing angle 123 may also include the examination area 104 and the imaging head module 111.

The imaging workstation additionally may include a computer 121 interfaced with at least one imaging sensor 115 and with some or all of the positioning structure locking means. The computer 121 and imaging sensor 115 may be interfaced using, for example, video, USB, IEEE1394 (A, or B), camera link Ethernet, etc., or any combinations thereof. Additionally, the computer 121 may interface with the display 110 to display images and data. Software may be installed in the computer 121, including modules for hardware control, image and data capturing, image processing, analysis and display, and image and data storage for retrieval and review.

As mentioned above, in some embodiments, the base member 101 may be a mobile base. The base member 101 may use one or more individually lockable castors. Additionally, at least one of the planar positioning structure 103, space micro-positioning structure 105 or the imaging head module 111 may be mounted directly on the base member 101. The space micro-positioning structure 105 may include a vertically telescoping columnar member ending in a pivoting structure 108. The imaging head module 111 may be affixed to the pivoting structure 108. As a result, the workstation itself may be mobile.

Thus, in some embodiments of the imaging workstation, the planar positioning structure 103 may be affixed to a mobile base, and the space micro-positioning structure 105 may be affixed to the planar positioning structure 103. In some embodiments, the base member 101 may be an immobile datum such as a floor or ceiling, or examination bed, and the planar positioning structure 103 can be mounted fixedly to the datum. In some embodiments, the space micro-positioning structure 105 can be affixed directly on to the base member 101 and the planar positioning structure 103 can be affixed to the space micro-positioning structure 105.

In one embodiment, the base member 101 includes an eccentric, ellipsoid shaped base-plate mounted on individually lockable wheels. Additional braking and stabilization members may be integrated in the base-plate. The braking and stabilization members may temporarily fix the base to the datum. The base member 101 may further consist of two tubular elements, one of which is fixed on to the base plate while the second rotates around the fixed tubular member with the help of a self lubricating bushing or a set of axial thrust bearings. The rotation of the tubular assembly may be limited to a maximum of 90° by a press-fit dowel pin moving in a machined groove. A vertical columnar member may be mounted to the fixed tubular member. The vertical columnar member may support a large format image display unit.

In some embodiments, the planar positioning structure 103 may be a movable structure, rotating (e.g., rotating at an angle Φ as shown in FIG. 1) around appropriately fixed and stable vertical members on the base member 101. The rotating part may include, for example, one or more roller bearings, a set of axial thrust bearings, or self lubricating bushings. Additionally, the planar positioning structure 103 may include an extension.

In other embodiments of the claimed workstation, the planar positioning structure 103 may be a mechanical slider (moving, for example, in the x plane in FIG. 1). The movable slider which may include a stable platform and a movable carriage which may be brought in close proximity to the target area to be examined. The motion may be accomplished, for example, with a movable carriage mounted on a closed circuit of rolling balls, rotating rollers moving on guide rails or bushing elements sliding on corresponding guide elements.

In other embodiments, the planar positioning structure 103 may be a wheeled trolley upon which other components are mounted. The trolley may include two platforms supported on columns. The first platform may serve as the mounting platform for other structures of the workstation, and the second platform may serve as the location surface of the wheels in the trolley. Additionally, the trolley wheels may be individually lockable. In some embodiments, the trolley may be collapsible. For example, the trolley may possess collapsible or telescoping columns.

The planar positioning structure 103 may be mounted fixedly at one end to the rotating tubular member. The planar positioning structure 103 may be a relatively long member with a vertically supporting foot, fixed near its other end, with a lockable, integrated wheel capable of swiveling through 360°. Following the range of motion allowed by the two tubular sections, the planar positioning structure 103 may rotate from its extended (rest) position, which allows the patient to access the examination platform 102, to its closed (imaging) position. In the course of this rotation, the planar positioning structure may translate the imaging head module 111 in close proximity with the examination area 104.

In some embodiments, the space micro-positioning structure 105 may be a linear translator working in the Cartesian space (x,y,z). The space micro-positioning structure 105 may include linear guide elements, which may be, for example, linear slideways or pillow blocks mounted on suitable guide rails. The linear guide elements may move on, for example, incorporated roller balls, cross-rollers or self-lubricating bushings.

In some embodiments, the planar positioning structure 103 and the space micro-positioning structure 105 include a multi-jointed articulating arm. The arm may position the imaging head module 111 with the use of horizontal and vertical rotational elements. These elements may be, for example, roller bearings of the axial thrust or rotational type, or self lubricating bushings, or a combination thereof. Additionally, the arm may be lockable at some or all of its articulating joints using, for example, pneumatic, electrical, mechanical, electro-magnetic or hydraulic means.

In some embodiments, the pivoting structure 108 is an axial joint having at least one degree of freedom, and may be mounted directly onto the planar positioning structure 103 or the base member 101. The at least one degree of freedom may provide the pivoting structure 108 with the capability of pitch, yaw or tilt. The pivoting structure may include a solid rod-like member to accomplish this motion.

In other embodiments, the pivoting structure 108 may be a ball joint structure attached to the planar positioning structure 103, the space micro-positioning structure 105 or to the base member 101. The ball joint may include a ball 810 and a suitable casing 810 to encase the ball, and suitable means for attaching the ball joint to the planar positioning structure 103, the space micro-positioning structure 105 or the base member 101.

FIG. 2 depicts an exemplary imaging head module 111 that may be employed in the imaging workstation. The imaging head module 111 may capture images from the examination area 104 and may provide illumination of the examination area.

The imaging head module 111 may also house suitable imaging and illumination optics and optomechanical elements for manipulating a light beam. Images may be captured with an imaging sensor 115, which may be, for example, a CCD, a CMOS imager or a combination thereof. The imaging sensor 115 may be configurable to capture images in color or black and white. The imaging sensor 115 may operate in conjunction with suitable imaging optics 112. Additionally, the imaging optics 112 may provide an imaging field of view substantially equal to the size of the examination area 104. Additionally, a light source 113 may provide illumination. The light source 113 may be mounted substantially at a right angle, or substantially parallel to the imaging sensor 115 and imaging optics 112, or at any angle in between. The illumination source may include suitable optical elements to focus the beam to provide an illumination spot 206 substantially equal to the imaging field of view and the size of the target area.

The imaging head module 111 may include beam manipulation optical elements to allow the imaging and illumination spots to substantially overlap, regardless of the angle formed between the imaging sensor 115 and light source 113. The beam manipulation optical elements may be, for example, a partly or fully reflective mirror element, a prism, a polarizing beam splitter, or a combination thereof. The light beam may be manipulated to illuminate the target examination area from, for example, a location above the imaging optics means. Manipulating the light beam in this manner may provide a shadow-free examination area so that the target area can be substantially illuminated.

The imaging head module 111 may include a means of dispensing a diagnostic marker. The means of dispensing a diagnostic marker may include a spray nozzle, full cone or hollow cone, and a means of pressurizing the marker before delivery to the spray nozzle. The pressurizing means may include, for example, a manual, pneumatic, or electrical mechanism such that sufficient back pressure can be built up at the inlet to the spray nozzle so that a proper spray pattern can be fully developed. The diagnostic marker may be stored in a container 402 pre-filled with the marker. The container 402 may be attached to the supporting and pivoting structures, or the marker may be introduced to the dispensing system at the moment of examination.

The imaging head module 111 may include a speculum 117 with an extension shaft temporarily attached to the imaging head module 111 for the duration of the examination in a releasable way. The extension shaft may be designed so that, when attached to the imaging head module 111, the imaging, illumination ray symmetry axes, and the agent dispensing pattern longitudinal axis become substantially collinear with the speculum's longitudinal axis 204. In this way, the imaging field-of-view, illumination from a light source 113, and the tissue area covered by the agent substantially overlap.

Additionally, the imaging module may include a first mechanical support 119 so that a speculum 117 and its extension shaft can be attached in a releasable way. The mechanical support 119 may also include means of attaching the diagnostic marker system. Additionally, the imaging module may include a second mechanical support 120 for permanently fixing the imaging head module 111 to the supporting structure.

In some embodiments of the workstation, the imaging head module 111 may be attached to the pivoting structure 108.

The imaging head module 111 may be configured so that a focused, shadow-free and glare-free tissue overview image can be obtained once the imaging head module 111 is connected with the speculum extension shaft 1010. To achieve such an image through the relatively small rear aperture of the speculum 117, suitably sized imaging and illumination elements may be employed. These imaging and illumination elements may be mounted in close proximity to the second mechanical support 120 so that their respective light spots substantially overlap on the examined area, without the corresponding light ray being obstructed by the speculum 117. The second mechanical support 120 may be affixed onto the first mechanical support 119, which may be detachably connected with the speculum extension shaft 1010 through a sleeve 1004. At the top of the sleeve 1004 may be an injector cap 1006. Fine focusing may be achieved through auto or manual focusing optics. Alternatively, fine focusing may be achieved through a linear translator that translates the first mechanical support 119 in relation to the second mechanical support 120 through a fine focusing knob.

For a realistic and complete documentation, and to facilitate treatment, the imaging workstation may be configured to provide stereo imaging. This can be accomplished by employing, for example, two imaging sensors, image focusing optics, and appropriate display means. Furthermore, the imaging workstations may be configured with two imaging sensors, one coupled with magnifying optics for imaging of the cervix, and the other with an endoscope probe to image the endocervix.

In some embodiments, the imaging sensor 115 in the imaging head module 111 may include, for example, a CCD camera, a CMOS camera, or a combination thereof. The cameras can provide color images or black and white images. Additionally, the imaging sensor 115 may have a spatial resolution of at least 640×420 pixels and the sensor data can be transmitted using a protocol such as video, USB, IEEE1394a, IEEE1394b, camera link, or Ethernet.

In some embodiments, the imaging head module 111 may include imaging optics 112 such as constant magnification optics, zoom optics, scalable magnification optics and endoscope optics. In some embodiments, the imaging optics 112 used in conjunction with the imaging sensor 115 may be a 25-35 mm lens or a zoom lens and may be of the type C-mount, CS mount or of any other mount type.

In some embodiments, the imaging head module 111 may include an illumination source 113 such as Xenon, Light Emitting Diodes (LED), Halogen, or any other light source that can emit light. In some embodiments, the emitted light is in the spectral range of 400 nm-700 nm.

Additionally, the imaging head module 111 may include first and second polarizers 207. The first polarizer may be placed in the imaging sensor's imaging path, while the second polarizer may be placed in the light path of the illumination source, with their polarization planes being substantially at right angles to each other. The polarizers may be placed in the paths by temporary or permanent means and may be adjusted to achieve the desired angle between their polarizations planes.

Furthermore, the imaging head module 111 may include a camera to image the vagina and the cervix of the uterus, while an imaging sensor 115 may be coupled with an endoscope for the imaging of endocervical canal and the endocervix.

In some embodiments the camera and the light source 113 may be mounted on the second mechanical support 120. The second mechanical support 120 may be mounted on the first mechanical support 119, which in turn may be mounted on the pivoting structure 108 through a linear translator. The linear translator may allow for fine focusing.

In some embodiments the beam manipulation optics 114 may include a light deflector 201 such as a prism, polarization beam splitters, dichroic mirrors, dichroic reflectors, fully or partially reflective mirrors, or combinations thereof. In some cases, the sizes of the imaging sensor 115 and the light source 113 preclude side-by-side placement. In these cases, light may be deflected so that the light rays of the imaging sensor 115 and the light source 113 become substantially coaxial with each other and with the speculum longitudinal axis 204 (when connected). The light deflector 201 may deflect the light of either the imaging sensor 115, or of the light source 113, or of both.

In some embodiments, the beam manipulation optics 114 include a planar mirror which is oriented so as to achieve coaxial illumination with the imaging field of view. The planar mirror may be supported on an off-center axis along its surface so as to be fixed in a desired position by fastener means or by permanent means. In some embodiments, the beam manipulation optics 114 may include a non-planar mirror, which is encased and held in a position to provide a coaxial illumination beam with the imaging field of view

In some embodiments, the beam manipulation optics 114 includes laser beam manipulation optics to manipulate a laser beam for image guided laser treatment. Beam manipulation may be carried out by altering the relative orientation of these elements with respect to the illumination source. The orientation may be altered by, for example, mechanical or electrical means. The orientation may be achieved using pre-determined coordinates or using electrical feedback for the imaging data from sources external to the imaging workstation. In some embodiments, the beam manipulation optics 114 include at least one mirror controlled with a joystick to manipulate a laser beam. In this case, the beam manipulation optics 114 may be driven by electrical drive means such as micro-motors, servomotors or stepper motors that interface directly with the joystick to achieve the desired orientation of the beam manipulation optics 114 and the laser beam.

In some embodiments, the beam manipulation optics 114 may be a set of galvanic mirrors to manipulate a laser beam for tissue treatment. The galvanic mirrors may be added in a retro-fit fashion to the imaging workstation.

In some embodiments, as depicted in FIG. 2, the light deflector 201 and the light source 113 are located on the same side of the central ray axis of the imaging means. Both the light deflector 201 and the light source 113 are positioned so as not to obstruct the field of the imaging means. At the same time, the light deflector 201 and the light source 113 provide illumination that interacts with the light deflector 201 and then becomes substantially coincident with the field of the imaging means at the surface of the examined tissue. This is accomplished by maintaining the light deflector 201 on one side of the central ray axis of the imaging means, but as close as possible to it, and positioning it at 45° to the central ray axis. Additionally, the light deflector 201 is also positioned at 45° to the central axis such that, until the light from the illumination source interacts with the light deflector 201, the central axis of the emanating light is at 90° to the central axis of the illumination means.

In some embodiments, the imaging head module 111 may include a heat sink 208. The heat sink may have a heat sink flange 209, spacers 210, a fan 211, and threaded shafts 212.

In one embodiment the imaging head module 111 includes an imaging sensor 115 and associated imaging optics 112. The imaging sensor 115 may be an at least one color CCD sensor of at least 1024×768 resolution coupled with an appropriate imaging lens of at least 20 mm focal length with a 20-35 cm working distance. The imaging lens may provide an appropriately sized field of view at a desired axial distance, and may provide variable and lockable aperture settings.

Additionally, the imaging head module 111 may include a light source 113, such as an LED light source, of suitable intensity and spectral range so that the light source 113 may cover at least the range of about 400 nm-700 nm. The light source 113 may work in conjunction with the color CCD. The light source 113 may also include suitable focusing optics for illuminating the imaging field of view. Additionally, the light source 113 may include a mechanism to allow beam manipulation to achieve coaxial illumination with the imaging field of view. In one embodiment, the imaging head module 111 includes the light source 113, positioned at substantially right angles to the CCD and the imaging lens. The beam output from the light source 113 is reflected towards the target area with the use of a suitable reflective mirror. Coaxial illumination within the imaging field of view may be achieved by manipulating the relative angle of the mirror, the relative angle of the light source 113, or both. Additionally, vertical adjustments to the position of the CCD and imaging lens may provide a coaxial field of view. The net result of the provided adjustments is that the illumination cone and the imaging cone may be substantially coincident.

In one embodiment, at least one of the imaging sensor 115 and the illumination means may be affixed to the second mechanical support 120, and the second mechanical support 120 may be affixed to the pivoting structure 108 through a linear slider, for fine focusing.

In one embodiment, the light deflector 201 is placed distantly enough from the imaging means such that the subjected light ray deflection forms a clear aperture, from which the light rays of the illumination means pass substantially unobstructed. In another embodiment, the light deflector 201 is placed distantly enough from the illumination means such that the subjected light ray deflection forms a clear aperture, from which the light rays of the imaging means pass substantially unobstructed.

In one embodiment, the CCD imaging sensor 115 is coupled with a polarizer 203 with a first orientation of its polarization plane. The light source 113 may be a white LED light source equipped with optical elements for light beam focusing on the examination area 104. In addition, the light source 113 may be coupled with a polarizer 203 with a second orientation of its polarization plane with the second orientation adjusted to become substantially perpendicular with the first polarization plan.

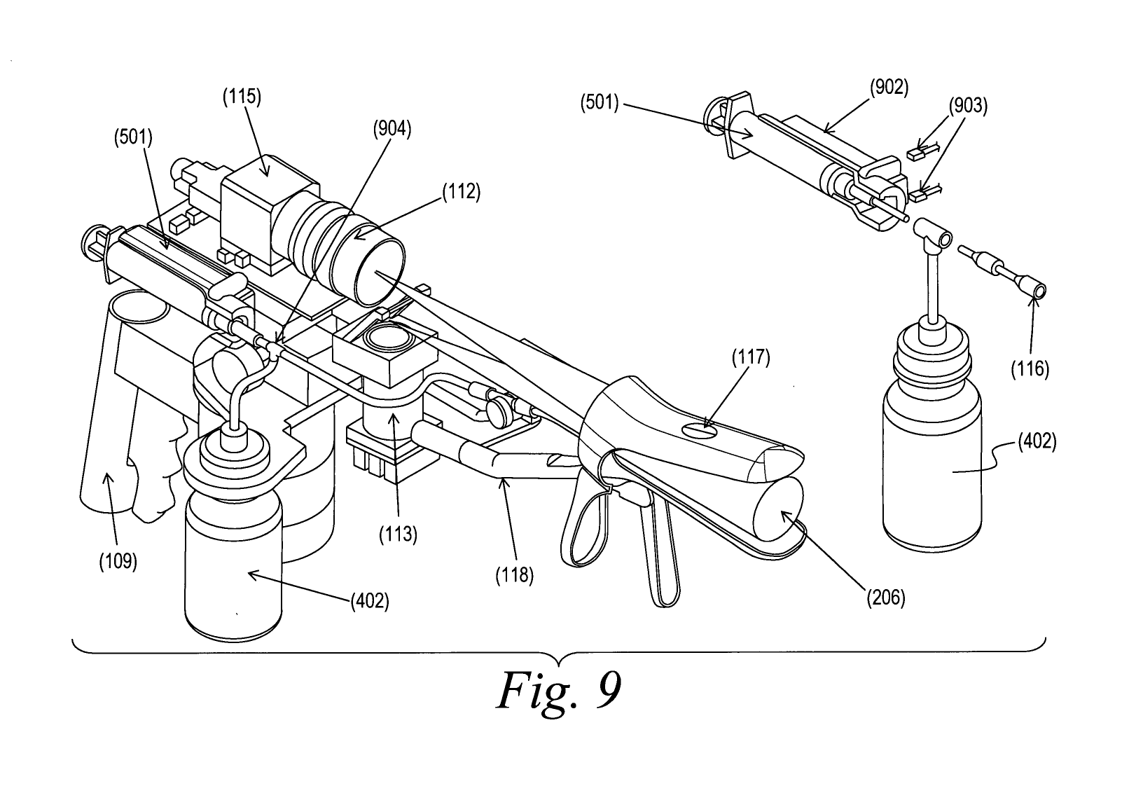

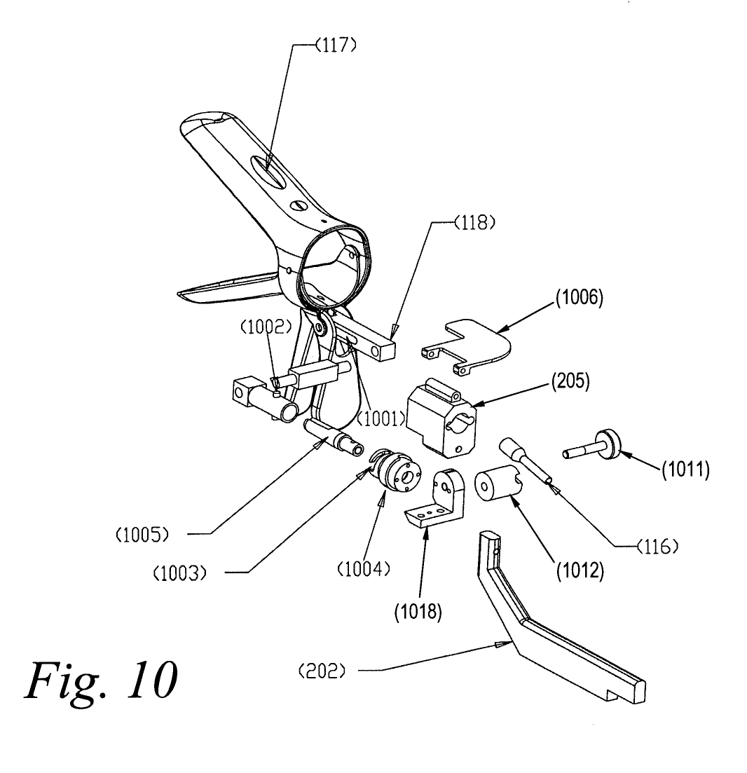

In one embodiment, the imaging head module 111 includes a diagnostic marker dispenser system. The system includes a container 402 mounted fixedly to an asymmetric bracket 401 with a suitable opening for supporting the container 402, located on top of a ball joint. The diagnostic marker dispenser system may further include a medical syringe of fixed capacity which is temporarily mounted in its dedicated holder mounted on the imaging head module 111. Furthermore, the syringe may be connected to the container 402 via a two way valve 904 (FIG. 9) affixed directly to the syringe. The second port of the two way valve 904 may be connected to a flexible tube terminating in a permanently bonded, narrow angle, full-cone axial spray nozzle. The nozzle may spray uniformly sized droplets of the diagnostic marker onto the target tissue area. Additionally, the nozzle may be aligned such that the spray cone of the nozzle is substantially coincident with the illumination and imaging cones. The nozzle may be fixed in a detachable way to the speculum attachment block to allow the nozzle to be changed while maintaining its position and angle of spray.

Additionally, the imaging head module 111 may include a mechanism to detachably attach a vaginal speculum 117 to it. The speculum 117 may be attached to a multi-member block via an extension bracket 202 fixedly attached to the asymmetric bracket 401. At the distal end of the extension bracket 202 a vaginal speculum 117 may be supported in a releasable way.

In one embodiment, the base member 101 includes a bayonet type mechanism including a sleeve 1004 with an incorporated angled groove 1003. A pre-load mechanism for the sleeve 1004, which in one embodiment consists of screw type, spring loaded balls, may allow an extension shaft at the back side of the vaginal speculum 117 to be locked into the sleeve 1004. The extension shaft attached to the speculum 117 may be substantially hollow and have a dowel pin 1002 pressed through it close to its distal end, perpendicular to the axis of the shaft. Inside the pre-loaded sleeve 1004 may be a receptacle 1005 for the dowel pin 1002 that guides the motion of the extension shaft and the speculum 117 without allowing for rotation. While engaged, the pin may be aligned with the opening in the angled groove 1003 in the sleeve 1004 and with the inner receptacle 1005. The provided lever may then be turned counterclockwise to force the dowel to move back along the receptacle 1005 through the distance governed by the angled groove 1003. Since the entire sleeve 1004 is pre-loaded using spring loaded balls, the effect is to provide a positive pressure between the dowel pin 1002 and the angled groove 1003 to prevent accidental release of the speculum 117 from the system. Additionally, both the extension bracket 202 and the speculum extension shaft 1010 may be designed so that the central axis of the speculum 117 may be coincident with the axis of the CCD and also with the axis of the imaging cone. Additionally, the speculum extension shaft 1010 may include a groove at around its midpoint shaped to follow the motion of the speculum 117, thereby spatially maintaining the axis of the speculum 117 and maintaining alignment with the CCD axis and the illumination cone.

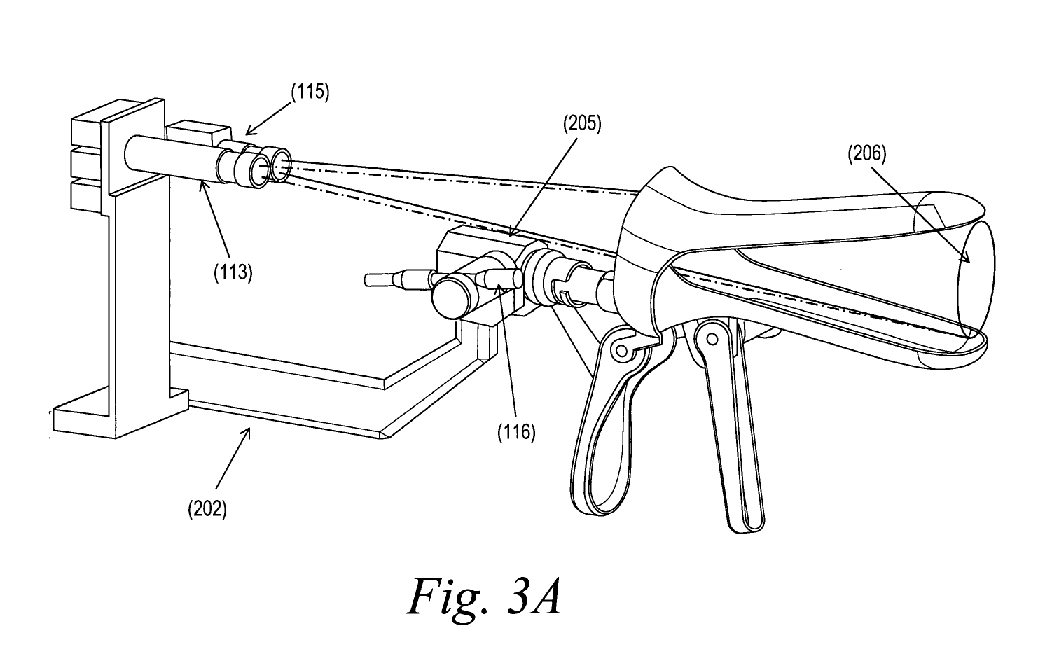

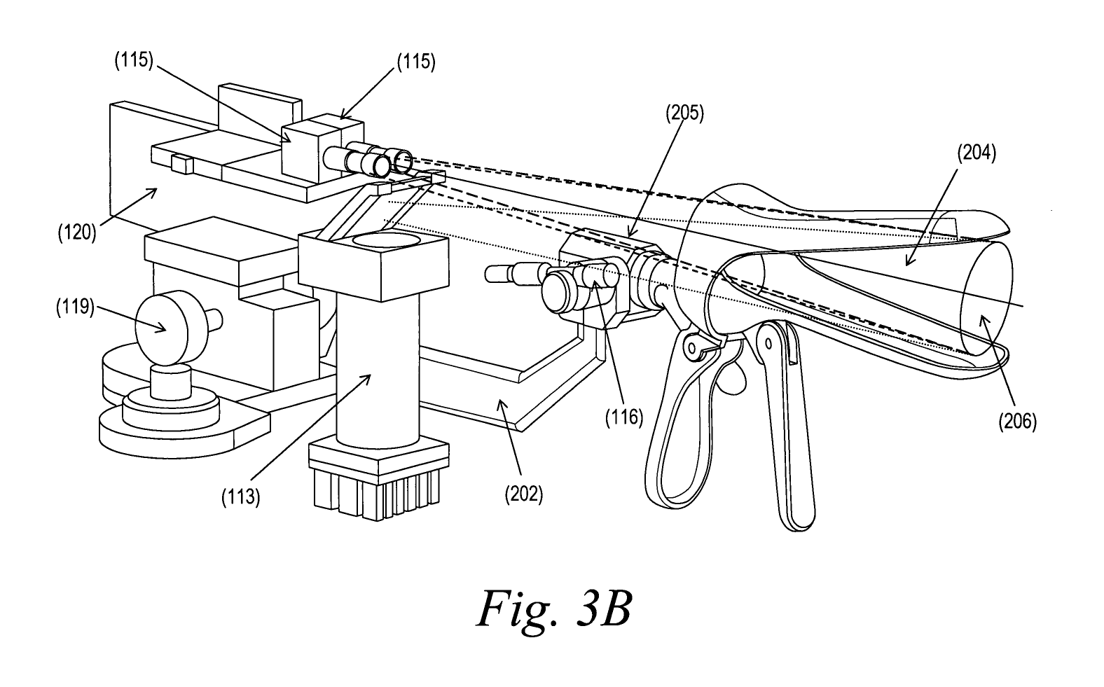

FIG. 3A depicts the imaging head module 111, where the imaging lens means is a microlens with a diameter less than 1 cm and is positioned parallel to the illumination source. This allows the imaging field of view and the illumination field to be substantially coaxial at the target area. This is achieved by the use of members in the illumination source that possess a size envelope similar to the microlens so as to be in close proximity with the imaging means.

FIG. 3B depicts the imaging head module 111 with two imaging sensors placed in close proximity to each other and coupled with the microlens of FIG. 3A. This configuration allows for stereo vision of the vagina and that of the cervix. The images may be displayed on display means providing stereo perception.

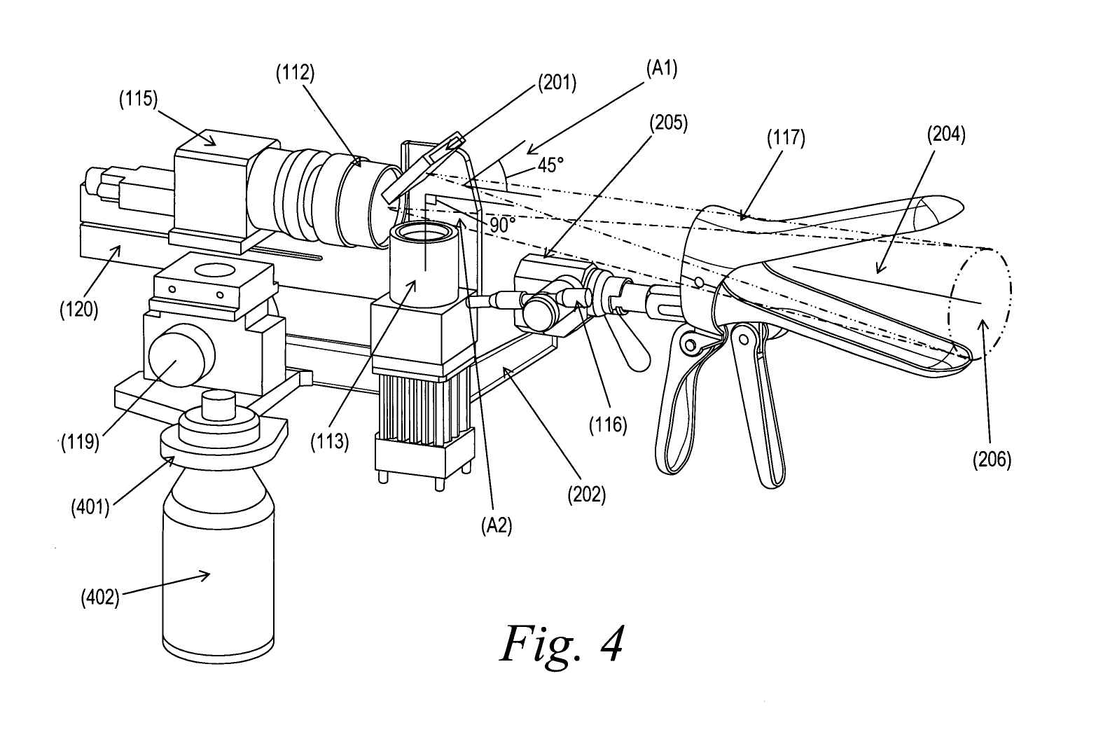

FIG. 4 depicts the imaging means and the illumination means placed at substantially right angles to each other within the imaging head module 111. Additionally, the beam manipulation optics 114 are at an approximately 45° angle to one of the axes of either the imaging means or of the illumination means. This has the effect of reflecting the rays incident to the beam manipulation optics 114 approximately 90°, thereby making the rays substantially parallel with the other axis.

In some embodiments, as depicted in FIG. 4, the light deflector 201 and the light source 113 are located on the opposite sides of the central ray axis from the imaging means. This is preferable in cases where the upper half of the rear aperture of the speculum 117 is wider. In this way, the entering light beam is not obscured. Both the light deflector 201 and the light source 113 are positioned so as not to obstruct the field of the imaging means. At the same time, the light deflector 201 and the light source 113 provide illumination that, after interacting with the light deflector 201, becomes substantially coincident with the filed of view of the imaging means at the surface of the examined tissue. This is accomplished by maintaining the light deflector 201 on one side of the central ray axis of the imaging means, but as close as possible to it. Additionally, the light deflector 201 is positioned on the opposite side of the central ray axis from the illumination means and at 45° to the central axis of the illumination module, such that until the light from the illumination source interacts with the light deflector 201, the central axis of the emanating light is at 90° to the central axis of the illumination means.

In some embodiments, the imaging workstation may also incorporate a mechanism for the uniform and standardized application of a diagnostic marker, such as acetic acid solution, onto the surface of the tissue. In cases where the recording of the dynamic optical phenomena, provoked by the marker, is required, means for synchronizing the initiation of the image capturing procedure with the completion of the marker application may also be included in the imaging workstation.

In some embodiments, the agent dispenser 116 may be a mechanism for dispensing the diagnostic marker onto the surface of the examined tissue. The mechanism includes an application probe, which may be a narrow angle full cone or hollow cone, an axial spray nozzle, a container 402 for the diagnostic marker, and a means for delivering the diagnostic marker from the container 402 to the application probe. Furthermore, the application probe may be fixed on a mount. The mount may be disposed directly or indirectly, through an extension bracket 202, at a position on the first mechanical support 119. The longitudinal axis of the application probe may be prefixed so that when the imaging head module 111 is connected with the speculum extension shaft 1010, the marker is applied substantially homogeneously onto a tissue area of at least equal size with illumination from light source 113 and the imaging sensor's field-of-view.

In some embodiments, the application probe may be mounted on a mechanical mount which includes a pre-aligned fixture for aligning the probe. The pre-aligned fixture is designed so that, when the probe is locked into the fixture, its orientation ensures a substantially homogeneous application of the diagnostic marker onto the examined tissue.

In some embodiments, the container 402 is a single compartment container. The container may be filled with a standardized volume of the diagnostic marker and delivered to the application probe using means appropriate for creating the necessary pressure and flow conditions required to effect the desired homogeneous application onto the examined tissue.

In other embodiments, the diagnostic marker container 402 is a dual compartment arrangement where the first compartment is a reservoir volume of the diagnostic marker and the second compartment contains a standardized fraction of the volume of the diagnostic marker. The two compartments may be connected via appropriate means, such as valves and pressure and vacuum creation members. Additionally, the agent dispenser 116 includes means of delivering the diagnostic marker from the second compartment to the application probe.

In some embodiments, the means for enabling the application of the diagnostic marker are manual. In these embodiments, manually delivered force is used for the creation of the requisite back pressure at the inlet to the application probe in order to create the desired spray pattern. The spray pattern may allow for the homogeneous application of the diagnostic marker onto the examined tissue.

In some embodiments, the means for enabling the application of the diagnostic marker are electromechanical and include drive components such as stepper motors and servomotors. These means may be connected directly or indirectly to a pumping mechanism such as a reciprocating positive displacement pump, peristaltic pump, centrifugal pump or diaphragm pump. The motors may be programmed and the pumps may be calibrated so as to deliver a standardized volume of the diagnostic marker to the inlet of the application probe flow conditions appropriate to develop the spray pattern so as to achieve the desired homogeneous application of the diagnostic marker onto the examined tissue surface. Additionally, the motors may be driven by an electrical signal which may be generated by the computer 121.

In some embodiments, an electrical signal is used to initiate image capture by the imaging means, and to synchronize the image capture with the end of the application of the diagnostic marker. The computer 121 may be programmed to record the completion of the application of the diagnostic marker, or may be pre-programmed to initiate the image capturing at a pre-determined time interval after the commencement of the application of the diagnostic marker.

In some embodiments, sensors are incorporated to detect the completion of the application of the diagnostic marker onto the examined tissue surface. The sensors may be, for example, optical sensors, capacitive sensors, proximity sensors, motion sensors, pressure sensors, flow sensors, displacement sensors, or a mechanical toggle switch. Activating the sensors may initiate image capture using the imaging means, thereby synchronizing the image capturing with the completion of the application of the diagnostic marker onto the examined tissue surface.

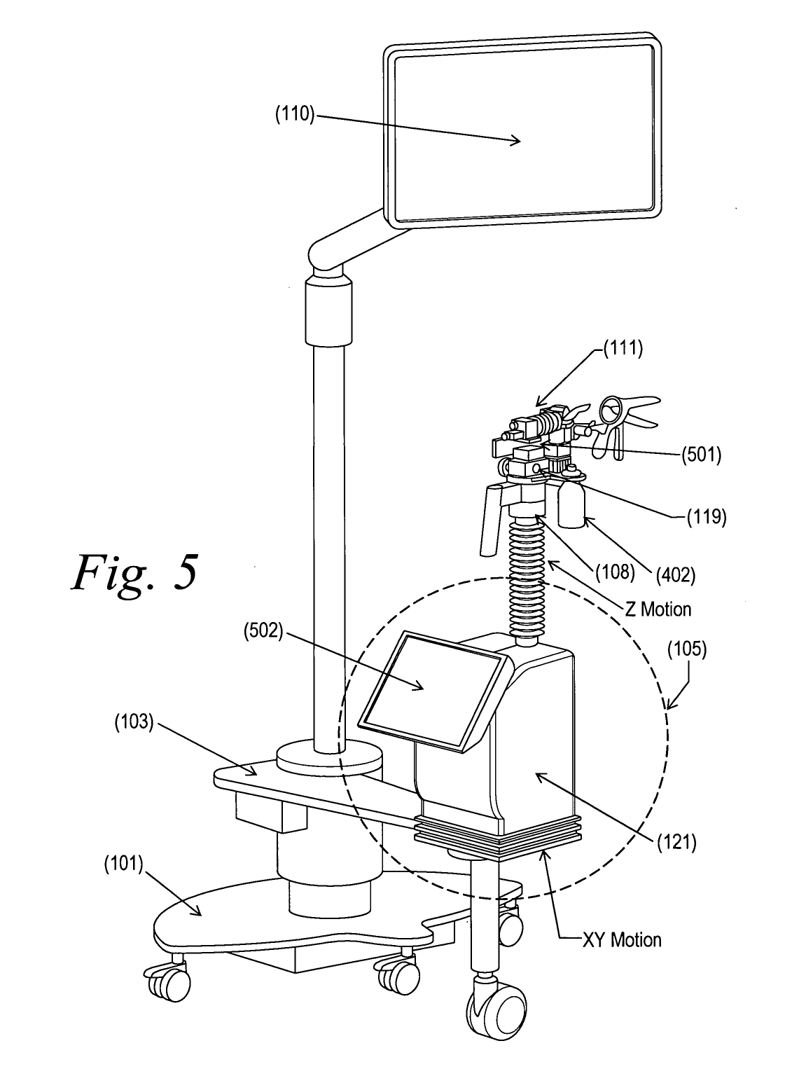

FIGS. 5 and 9 depict the manual means for delivering the diagnostic marker to the application probe, which include a manually depressible syringe-type mechanism 501. The end of the syringe-type mechanism 501 may be detachably connected to the application probe. Manual force may be used to depress the syringe plunger and create the requisite back pressure at the inlet to the application probe in order to achieve the desired homogeneous application of the diagnostic marker onto the examined tissue surface. In some embodiments, the syringe-type mechanism 501 includes an integrated piston.

In some embodiments, the syringe-type mechanism 501 includes an integrated piston with an opaque and air tight end. Furthermore, the syringe-type mechanism 501 may be supported on a structure that fully or partially covers the container 402 of the syringe-type mechanism 501 along its length. Moreover, the structure may include a sensor to detect motion of the moving parts in the syringe-type mechanism 501. Additionally, the sensor may be a combination of a light source 113 and a photo-sensor 903 (FIG. 9) which may be of the normally on (NO) type. Furthermore, manually depressing the plunger of the syringe-type mechanism 501 may cause the interruption of the photo contact between the light source 113 and the photo-sensor 903 by the opaque and air tight end, causing a triggering signal to initiate the image capturing process.

The syringe-type mechanism 501 may be supported on a structure that fully or partially covers the container of the syringe-type mechanism 501 along its length. Furthermore, the sensor may be a pair of electrical contacts that are brought together when the depressing action of the plunger of the syringe-type mechanism 501 is completed. The electrical contacts may be brought into contact using a mechanical toggle switch or any other means, and the contacting of the electrical contacts may generate a triggering signal to initiate image capture, so as to synchronize the image capture with the end of the diagnostic marker application.

In some embodiments, the photo-sensor is located directly on the diagnostic marker container or is otherwise appropriately placed so as to detect the motion of the moving parts of the described manual means of application of the diagnostic marker. In some embodiments, the photo-sensor may be located on mechanical supports or structures that hold all or part of the diagnostic marker container. These mechanical supports may include mechanical brackets, plastic housings or other such encapsulations and supports as required for the support of the diagnostic marker container.

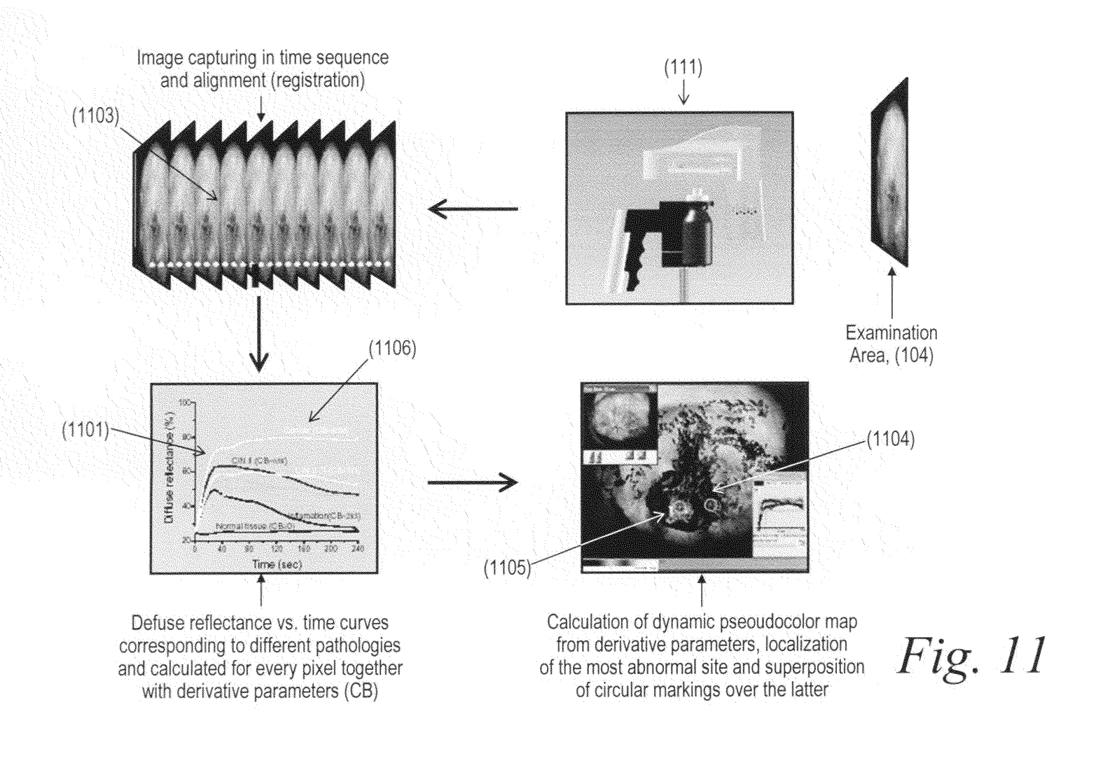

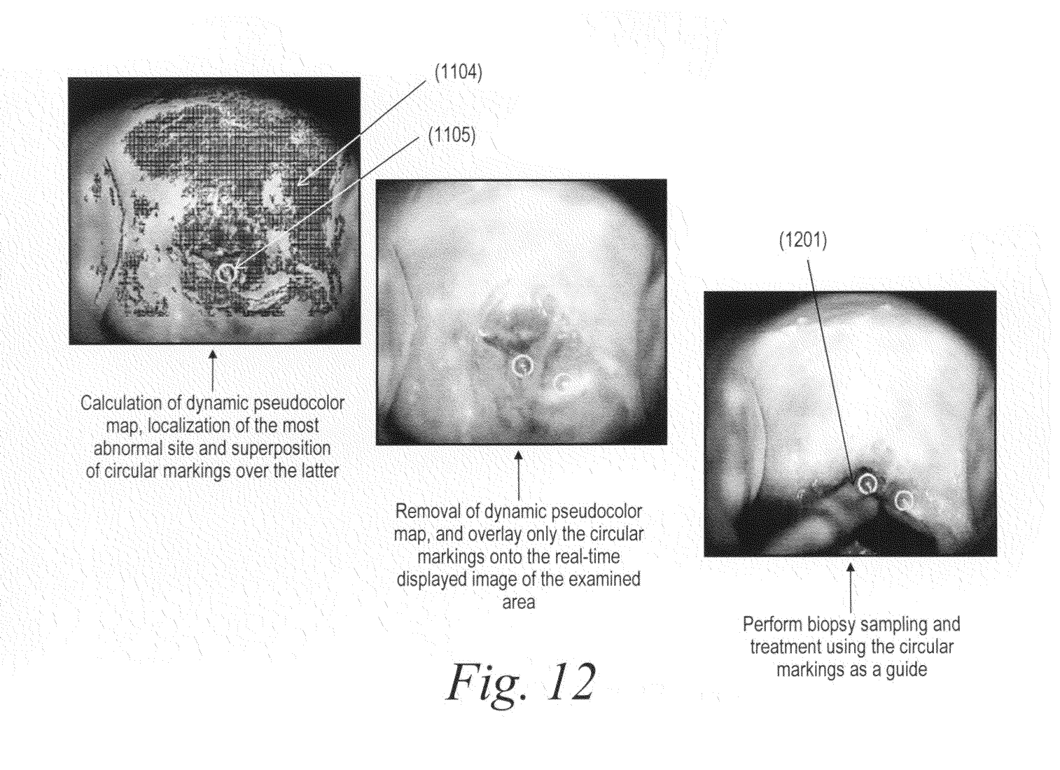

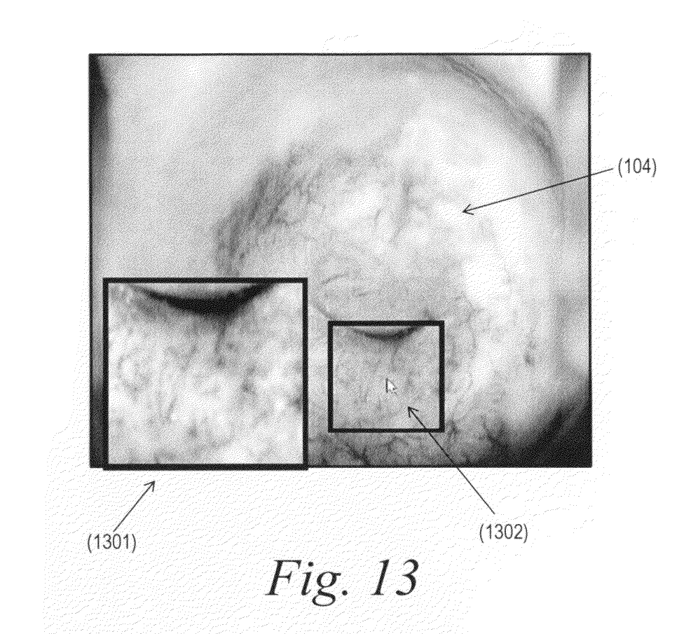

Imaging dynamic phenomena requires imaging sensor's field-of-view to be maintained in a substantially stable manner for prolonged examination periods. The imaging workstation includes means for such mechanical stabilization. In addition, the disclosed workstation corrects image motion artifacts occurring within the field-of-view by integrating image registration algorithms 1103 (FIG. 11). In some embodiments, stabilization is achieved by detachably connecting the imaging head module 111 with the speculum 117 equipped with an extension shaft. Once the connection is established, the supporting and pivoting structures may be locked to further secure stabilization and to support the weight of the speculum 117.

This connection also allows for the reproducible and uniform application of the diagnostic marker. Mechanical stabilization means may include a bayonet mechanism, spring loaded wedge shaped pins, or positive engagement spring loaded couplings. The bayonet mechanism may include a spring preloaded probe and the speculum extension shaft 1010 may be a female shaft designed to accept the probe. The wedge shaped pin mechanism may include an eccentric wedge pivoting around a fixed pivot and preloaded with a leaf spring, and the extension shaft may be designed to accept the wedge feature in it when properly aligned. A spring loaded coupling may be used that is preloaded both axially and radially so as to securely lock the speculum extension shaft 1010 in the coupling while facilitating the release of the shaft when the radial spring is released.

In some embodiments, the speculum 117 is detachably affixed to the imaging head module 111 with an extension shaft. The shaft may be coaxial with the central axis of the imaging means incorporated in the imaging module head. Additionally, the shaft may be attached to the imaging module head with semi-permanent means, such as mechanical locking means, magnetic means, electromagnetic means and pneumatic means.

In some embodiments, the computer 121 includes components and modules for interfacing with at least one of the imaging sensor 115, the user interface, the display 110, and the agent dispenser 116. In some embodiments, the computer 121 is mounted directly on the supporting structures.

The user interface may be a keyboard, a mouse, a track ball, voice interface, touchscreen 502, or a foot switch. In some embodiments, the user interface is located on the supporting structures. In other embodiments, the user interface is located directly on the computer 121.

Additionally, the computer 121 may include interface means to interface with printers, local networks and the Internet, either in a wired manner or wirelessly. In some embodiments, one of the interface means is wireless and may comprise of Bluetooth 1.2, Bluetooth 2.0, Infrared or any other protocol for wireless data transfer.

In some embodiments, the display 110 may be, for example, a head mounted display, video goggles, a touchscreen 502, or a projection display. In some embodiments, the display 110 may be a monitor that is mounted on a stand. The stand may be located on the supporting structures. In other embodiments, the stand is located on the base member 101 and is placed on one side of the examination bed, outside the angle subtended by the patient's legs. In other embodiments, the stand is located on the planar positioning structure 103.

The display 110 may be located within the viewing angle 123 of the user. The viewing angle 123 may also include the examined area. This allows the user to visualize both the examined area and the displayed image without moving their head.

In one embodiment, the computer 121 is based on a multiple core microprocessor with different cores handling different tasks in parallel. The computer 121 may include control means for controlling at least the locking mechanisms and for synchronizing and triggering image capture with agent application. The computer 121 may include computer memory and hardware interfaces for connecting computer peripherals, such as displays, user interface means, local network, hospital data bases, the Internet, and printers. Additionally the user interface may be a touchscreen 502, a keyboard, a wireless keyboard, a voice interface, a foot switch, or combinations thereof. The computer 121 may further include, mother board and graphics cards to support and carry out the various processes required to conduct the examination

The computer 121 may control the activation and deactivation of the space micro-positioning locks. Additionally, the computer 121 may be designed to take captured images from the optical head module, process the images using certain algorithms, and display the results on the display 110. The computer 121 may also include a touchscreen 502 user interface that may also display images while acting as a data entry or user interface point.

In one embodiment, software means enable the computer 121 to perform image calibration, image capturing initialization, image registration 1103 (FIG. 11), dynamic curve calculation, processing and analysis, dynamic pseudocolor map calculation and segmentation, biopsy sampling/treatment guiding documentation, image magnification, data base for storing, retrieval and post-processing of images and data.

FIG. 6 depicts an exemplary space micro-positioning structure 105 suitable for use in the imaging workstation disclosed herein. In one embodiment, the space micro-positioning structure 105 works in Cartesian coordinates. Motion may be provided in the XY-plane using two sets of guide elements in each direction, working on a set of three parallel equal-sized plates. The guide elements may be linear roller-ball type guide elements, linear cross-roller guide elements, linear self-lubricating bushing elements, or a combination thereof. In one embodiment, the resulting motion is substantially unrestricted and frictionless. Motion along the Z-axis is provided by a linear guide element 602 which is comprised of a splined, non-rotational shaft moving along a closed circuit of roller balls. The top end of the splined shaft 601 may terminate in a ball 810 (FIG. 8) fixedly attached to the shaft 601. The Z linear guide element 602 may be supported on a support member affixed to columnar structures mounted on the top plate 606 of the three plates used for affecting the XY motion.

In one embodiment, the space micro-positioning structure includes suitably sized constant force springs 603 mounted on the support member and affixed permanently to the splined shaft 601. The constant force springs 603 may rotate on a substantially frictionless drum and shaft which may be needle bearings or hardened steel shafts.

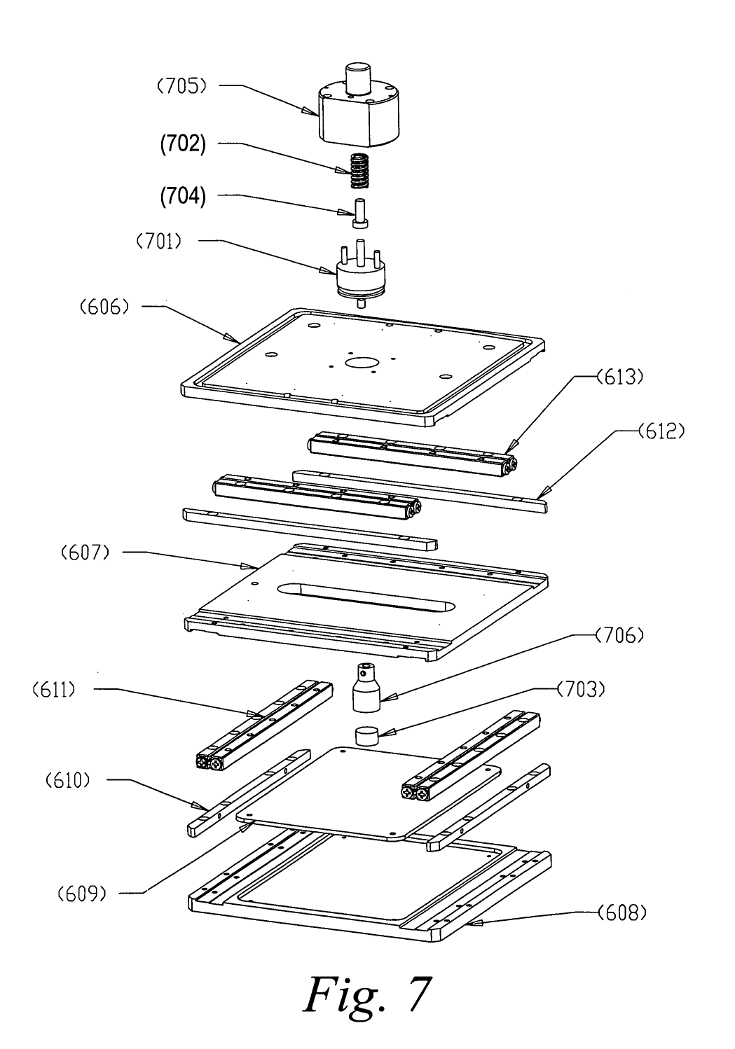

FIG. 7 depicts a way to temporarily fix the space micro-positioning structure 105 along its axes of motion. The space micro-positioning structure 105 may be temporarily fixed along all its axes of motion, X,Y, and Z. The X and Y motions may be temporarily fixed by stopping the relative motions of the top plate 606 and the bottom plate 608 with respect to each other. The top plate 606 may sit atop middle plate 607. One device suitable for stopping the X and Y motion is a brake made up of an electromagnet 701 and a suitably sized, helical counteracting spring 702 pressing on a friction element 704. The brake may be of the normally open (NO) type, may be engaged at all times, and may be released by action of the user. The action serves to activate the electromagnet 701 which retracts the friction element 704 mounted at the distal end of a suitable, ferro-magnetic mount. A brake pad holder 706 may sit atop friction element 704. Between the bottom plate 608 and the friction element 704 may be a stainless steel plate 609.

The bottom plate 608 may include a side sliders cover 610 for sliders 611. Further, the middle plate may include side sliders cover 612 for sliders 613.

Referring back to FIG. 6, motion along the Z-axis may be fixed temporarily using, for example, a motion drive element consisting of a stepper motor 605 and a timing belt 604 attached fixedly to the splined shaft 601. The motion drive element may be of the normally closed (NC) type and provides holding torque to the stepper motor 605, thus preventing the motion of the splined shaft 601. The circuit may be opened, and the motion released, using the same user action as for releasing the XY brake.

FIG. 8 depicts the pivoting structure 108 of an exemplary imaging workstation. In one embodiment, the pivoting structure 108 is a limited ball joint providing unlimited rotational motion, limited pitching motion, and zero tilting motion. The ball joint may use, as its central member, the ball 810 affixed permanently to the top end of the splined shaft 601. The ball joint may include upper, middle and lower disc-shaped members. The middle and the lower disc-shaped members may include a complimentary concave space, and may be interconnected by a pair of parallel rod members passing through the bodies of the middle and lower disc-shaped bodies through respective openings, trapping and thus restricting the ball 810 of the ball-joint among the middle disc-shaped member 805, the lower disc-shaped member 806, and the pair of parallel rod members.

The lower disc-shaped member 806 may act as a motion limiter, as it limits the motion of the ball joint when approaching the middle disc-shaped member 805 and traps and immobilizes the ball 810 between the two disc-shaped concave members. Additionally, the lower disc-shaped member 806 may restrict the motion of the ball joint with respect to the splined shaft 601 by creating a linear slit in the lower disc-shaped member 806 that acts as the entry point of the splined shaft 601 into the ball joint. By virtue of this slit, limited pitching is allowed and no tilting is allowed to the ball joint.

The upper disc-shaped member 804 may be affixed on top of the middle disc-shaped member 805. The parallel rod members, passing through respective openings in both the middle disc-shaped member 805 and lower disc-shaped member 806, terminate in the upper disc-shaped member 804. A pair of suitably sized helical springs may be mounted coaxially with the parallel rod members, and may be encapsulated between the upper disc-shaped member 804 and the middle disc-shaped member 805. The end of the parallel rod members may be secured using threaded fasteners 814 housed in suitable cavities in the lower disc-shaped member 806. The parallel rod members may be joined together using a suitable shaft so as to maintain the rod members relatively congruent to each other and for depressing the helical springs upon the action of a cam 807.

The cam 807, which may be an eccentric cam, may be housed and permanently affixed at one of its ends to the upper disc-shaped member 804. The cam 807 may have a suitable surface for depressing the shaft connecting the parallel rod members. A suitably shaped lever 811 may be in contact with the free end of the cam 807, and may have a corresponding follower path created at the end contacting the cam 807. The lever 811 may be housed in suitably designed casing 813.

A mechanism for transmitting a signal for the motion release of the space micro-positioning structure 105 or the planar positioning structure 103 may also be mounted along the lever 811. The mechanism may be activated when the lever 811 is depressed. In one embodiment, this mechanism is a microswitch, including a handle lower plug 815 and a microshalter 816, which transmits an electrical signal to the respective motion locking members in the positioning structure. Additionally, depressing the lever 811 and activating the incorporated cam 807 also has the effect of depressing the incorporated helical springs in the ball joint, thereby creating a separation between the lower disc-shaped member 806 and the middle disc-shaped member 805 comprising the ball joint. This has the effect of releasing the motion limits on the allowed degrees of freedom in the ball joint. The lever 811 and its casing 813 further may act as a handle 109 to allow for the manual positioning of the positioning structures upon release of the motion limiting members.

The imaging workstation may provide high, user independent performance by allowing the quantitative assessment of dynamic optical phenomena from a tissue surface. The dynamic optical phenomena may be generated after the application of diagnostic markers, such as acetic acid solution, onto a tissue surface. These markers may alter the optical properties of the tissue in a transient fashion, and with an effective marker the reliable and reproducible assessment and mapping of the dynamic optical characteristics could provide a means to improve the diagnostic performance up to a standardized base line. Clinical trials have shown that, when acetic acid solution is used as diagnostic marker, the calculation of Diffuse Reflectance (DR) 1101 (FIG. 11) versus time curves and derivative dynamic optical characteristics provide a means for improving the diagnostic performance and for standardizing colposcopic procedures. For example, it has been found that the time integral (i.e., the area under a curve) of the DR versus time curves taken over four minutes can provide a reliable cut-off value for discriminating low- from high-grade cervical neoplasia. It is therefore desirable to provide a means for reliable calculation of dynamic optical characteristics and parameters in order to eliminate artifacts due to tissue motion, and to eliminate noise factors that may be introduced during the measurement of the dynamic optical characteristics.

According to one embodiment, the imaging workstation integrates software for enabling unit control, performing acquisition of cervical images, and processing and analysis in a standardized, user independent fashion. The imaging workstation may allow quantitative monitoring, analysis, and mapping of the AW effect. In addition, the imaging workstation may provide means for digital image magnification and enhancement, further improving diagnostic capability. Both hardware and software of the workstation enable a method for standardized examination of the cervix.

FIG. 11 depicts some of the capabilities of an exemplary imaging workstation according to the teachings disclosed herein. The imaging workstation may perform image calibration, image capturing initialization, image registration 1103, dynamic curve calculation, processing, and analysis, dynamic pseudocolor map calculation and segmentation, biopsy, sampling or treatment guidance and documentation. The imaging workstation may have an image magnification module, and a data storage and retrieval data base.

The image calibration may assist in providing reproducible device-independent image acquisition through an interactive procedure for color balancing. The image calibration may further compensate for the variability in light intensity remitted by the tissue surface using image brightness control.

The image acquisition system may be calibrated using a graphical user interface. In one exemplary calibration method, a calibration plate with known reflectance characteristics is placed in the filed-of-view of the imaging sensor 115. The light source 113 then illuminates the calibration plate. The imaging sensor 115 records images and data corresponding to sub-areas of the calibration plate. The image acquisition system may regulate imaging parameters such as: grey values; red, green, and blue channels; brightness; and shutter. This regulation may continue until the output readings of the imaging sensor 115 reach desirable levels corresponding to the reflectance characteristics of the calibration plate. The regulated values of the imaging parameters may then be stored in the computer 121 memory means. The regulated values may be used as default values for subsequent examinations.

In some embodiments, the image calibration may be performed manually using scroll bars, for example, as part of a graphical user interface. Output readings of the imaging sensor 115 may be displayed on the display, and the scroll bars may regulate the imaging parameters using the output readings as feed-back. In other embodiments, the regulation may be performed automatically by the computer 121, using the output readings of the imaging sensor 115 as feed-back. In yet other embodiments, the regulation may be performed automatically by the computer 121, using the output readings of at least one optical sensor placed in the light path of the light source 113 as feedback.

It may be desirable to capture a reference image just before the application of the diagnostic marker (e.g., acetic acid solution) and to initiate the snap-shot imaging just after the application of the diagnostic marker. This may allow reliable quantitative monitoring of the AW effect. One exemplary sequence for achieving these ends may include capturing and storing a reference image in the computer memory means of the computer 121, applying the diagnostic marker, and capturing and displaying images in a time sequence, at predetermined time intervals for a predetermined duration.

During this sequence, the workstation may be set to a stand-by mode. A reference image may be captured and stored in the computer memory means of the computer 121. A subsequent reference image may be captured and stored, replacing the previously stored reference image in the computer memory means. This process of capturing and storing reference images may be repeated as long as the imaging workstation remains in stand-by mode. An electrical signal may be used to synchronize the initiation of the image capturing procedure with the end of the stand-by mode. The electrical signal may be generated when the application of the diagnostic marker is completed. Generating the electrical signal may cause the most recently captured image to be used as the reference image. At that point, images may be captured and displayed in time sequence, at predetermined time intervals for a predetermined duration.

In some embodiments, the predetermined time intervals are between about 1.5-5 minutes. In other embodiments, the predetermined time intervals are variable. For example, the time intervals may be short at an early phase of the acquisition process and longer at a later phase of the acquisition process.

It may also be desirable to align the acquired images in time sequence so that the AW effect may be quantitatively and reliably monitored. This may be a prerequisite for per-pixel calculation of dynamic optical characteristics and parameters. In order to substantially align the acquired images, the relative positions of the imaging sensor 115 and examination area 104 may be stabilized. This may be accomplished with the opto-mechanical arrangements described above. Even with these arrangements, however, micro-movements (for example, breathing, tissue contractions etc.) could cause in erroneous results. This problem may be addressed with image registration 1103. These image registration 1103 may compensate for misalignments caused by micro-movements occurring during the image acquisition procedure.

Image registration is the process of determining the point-by-point correspondence between two images. In one embodiment, the image registration 1103 may be an automatic image-based nonlinear (deformable) registration method. The reflectance images of the cervix may be captured in time sequence and registered using such a registration method. When a new image is available during image acquisition, it is registered to the previously acquired image. In this way, each image may be registered relative to a reference image.

The captured images may be compared at different time intervals, so that translational relative movements of sequential images may be determined. This may be accomplished, for example, using rigid registration algorithms. Images with excessive relative movement may be rejected. Further, relative movements due to tissue deformation in rigid-based registered images may be determined using deformable registration algorithms. Images with excessive deformations may be rejected. The registered images may be stored in computer memory means.

In some embodiments, image registration 1103 is performed in parallel with image acquisition in order to reduce the time required to process the imaging data. This also serves to reduce the examination time.

In some embodiments, image registration 1103 may be performed with reference a reference image for documentation purposes. The reference image may be the first image in a set of two images. The reference image may be held unaltered. A second image in the set of two images may then be a target image. The target image may be re-sampled in order to be registered to the reference image.

In some embodiments, image registration may be performed with reference to the last acquired image.

The images may be preprocessed to remove noise. Preprocessing the images may involve image improvement methods, such as noise removal and feature enhancement. Noise removal may be achieved using the Median Filtering method. In this method, the intensity of each pixel of the image is replaced by the median intensity in a circular window having a radius of three (3) pixels. Image enhancement may be achieved by subtracting a background from each image. The background image may correspond to the zero scale wavelet transform computed with, for example, the Atrous algorithm.

In some embodiments, these methods apply to those images that will be used for registration, but not the original images or the images displayed on the display for diagnostic purposes.

In some embodiments, image registration may be performed using rigid-body registration. The target image may be registered to the reference image, and a transformation function that determines the correspondence between all points of the two images is estimated. Given the coordinates of N corresponding points in the reference and target images,

{(xᵢ,yᵢ),(Xᵢ,Yᵢ): i=1, . . . , N},

a transformation function f(x,y) with components fₓ(x,y) and f_{y}(x,y) is determined such that the transformation function f(x,y) satisfies the conditions

Xᵢ=fₓ(xᵢ,yᵢ),

Yᵢ=f_{y}(xᵢ,yᵢ), i=1, . . . , N

Once f(x,y) is determined, then given the coordinates of a point in the reference image, the coordinates of the corresponding point in the target image can be computed.

In the frame of the rigid-body registration procedure, it may be that the transformation function is linear and represents global translational and rotational differences between the two images. In that case, the transformation function can be defined by:

$$\begin{matrix}X = x \cdot cos θ + y \cdot sin θ + {t}_{x} \\ Y = - x \cdot sin θ + y \cdot cos θ + {t}_{y}\end{matrix} \} ⇔ ( \begin{matrix}X \\ Y \\ 1\end{matrix} ) = ( \begin{matrix}cos θ & sin θ & {t}_{x} \\ - sin θ & cos θ & {t}_{y} \\ 0 & 0 & 1\end{matrix} ) \cdot ( \begin{matrix}x \\ y \\ 1\end{matrix} )$$

where θ represents rotational, and tₓ, t_{y} represent translational differences between the images.

These parameters can be determined if the coordinates of two corresponding points in the images are known. However, considering that the determination of the correspondence of two points may be noisy or inaccurate, multiple points may be used. In order to refine the transformation parameters to better align the features present in the images, all pixels whose value is not below a threshold value may be selected. In that case, the problem to be solved is an optimization problem with 3 parameters: two translations and one rotation. The simplex optimization method may be used in order to maximize a similarity metric that accurately represents image alignment. Simplex offers good convergence behavior and a good behavior for local minima.

As a similarity metric for the optimization, several different measures can be utilized. Two such measures are the spatial-frequency characteristics computed using the Fast Fourier Transform, and the Normalized Mutual Information.

The spatial-frequency characteristics of two images can be used as a similarity metric. In order to compute the spatial-frequency characteristics of the images the Fast Fourier Transform (FFT) can be adopted. Low-order transform coefficients measure low-frequency contents in an image and high-order coefficients reflect high-spatial frequencies present in an image. The method may provide good results for determining translational differences. Thus, the method may be used as a first step in the rigid-body registration algorithm, for determining a first approximation for the simplex method.

An alternative similarity metric is the Normalized Mutual Information (NMI) that explores the statistical dependence of images. NMI is appropriate for handling noise and occlusions. Given template fₜ[ ] and window f_{w}[ ], Pₜ(a) is the probability that the intensity at a pixel in fₜ[ ] is a and P_{w}(b) is the probability that the intensity at a pixel in f_{w}[ ] is b. When the template overlays the window, the probability that the intensity a in the template lies on top of the intensity b in the window is equal to their joint probability P_{tw}(a,b). If the template and the window truly correspond to each other, their intensities will be highly dependent and they will produce high joint probabilities. However, if the template and the window do not correspond to each other, they will produce small joint probabilities. The Normalized mutual information is computed as follows:

$$Y ( t , w ) = \frac{H ( t ) + H ( w )}{H ( t , w )}$$

Where H(t), H(w) represent the entropies of images t,w to be registered, and H(t,w) represents the joint entropy of t, w.

Rigid-body registration adopts a multi-resolution approach in order to reduce the computation time and avoid local minima. This approach computes similarity and optimization information for various image scales. Research has shown that mutual information produces a sharper peak at the best-match position, and thus is more suitable for sub-pixel registration of images than the correlation coefficient.

The algorithm for determining the Transformation Function can be pseudo-coded as follows:

Initial Estimate R₀ based on acquisition and FFT.

For scale 0 to n do begin

Initial Estimate R₀ computed from previous scale

Until “THE RESULTS ARE SATISFACTORY”

Compute NMI(Rᵢ)

Compute 3 new rigid parameters according to optimizer

END UNTIL

Thus, if the Transformation Function is determined, then given the (x,y) coordinates of a point in the reference image; the (X,Y) coordinates of the corresponding point in the target image can be determined. By reading the intensity at (X,Y) in the target image and saving it at (x,y) in a new image, the target image is point-by-point resampled to the geometry of the reference image. Although (x,y) are integers, (X,Y) are floating point numbers. Thus, the intensity at point (X,Y) has to be estimated from the intensities of a small number of surrounding pixels. An appropriate method for estimating the intensity at a point (X,Y) based on, for example, a 4×4 neighborhood is the Cubic splines method.

After the performing rigid-body registration of the images, the deformable registration follows. Because the cervix is live tissue, the images to be registered often have nonlinear geometric differences that cannot be corrected using the rigid-body registration. Thus, it may be more appropriate to use a nonlinear transformation function that may accurately register different parts of the images. In this case, one option is to use the Thin Plate Spline Transformation (TPS) function. TPS can be combined with robust similarity measures and local motion tracking algorithms. It does not require regular distribution of control points and allows for space-variant control-point density based on local image characteristics. The TPS transformation function can be determined by searching for local image characteristics and establishing point correspondences. In order to achieve this, the image is divided into a number of blocks. The upper left corner of each block defines one control point. Initially, the homologous points are determined based on the results of the rigid transformation. A template matching algorithm may be used to refine the pairs of homologous points and establish the final correspondence. Once homologous points are established, a closed-form solution of the TPS can be found. A linear system with a large number of parameters may be solved for each dimension. As in the case of the rigid body, singular value decomposition (simplex) may be used for solving the linear system in order to obtain robust and numerical stable solutions.

Images with excessive displacements and deformations may be rejected based on the results of the rigid and deformable registration. The decision may be made based on the translational and rotational differences. For instance, an image may be rejected if more than a predefined number blocks exceed certain limits. If it is decided that an image should be rejected, then it is exempted from the time sequence and from further processing.

The imaging workstation may further provide a reliable, artifact-free quantitative assessment of the DR vs time curves and associated parameters. If the line shape of the DR vs time curves is distorted, it may result in an erroneous calculation of derivative parameters, which may in turn result in false positive or false negative diagnoses. Aside from the motion artifacts, which may be eliminated with image registration algorithms, other factors may distort the line shape. These factors may include the generation of foam after the application of the diagnostic marker, or the presence of blood or mucus.

In order to overcome these factors and provide a reliable, artifact-free quantitative assessment of the DR vs time curves and associated parameters, a number of steps may be performed. The defuse reflectance vs. time curves may be calculated for each spatial location from images captured and stored in time sequence, before and after the application of the diagnostic marker. The defuse reflectance vs. time curves may be displayed during and after acquisition. The defuse reflectance vs. time curves may be smoothed using algorithms such as Butterworth, Fast Fourier Transformation, single and multiple exponential fitting based filters, difference based filters, or combinations thereof. A part, or all, of the defuse reflectance vs. time curves may be fit using the functions such as single and multiple exponential fitting, polynomial, or combinations thereof. A group of parameters may be calculated from the defuse reflectance vs. time curves. These parameters may include a time integral calculated for at least a part of the predetermined time duration of the acquisition process, maximum, time-to-max, and defuse reflectance curve slopes. The parameters may be compared with predetermined cut-off values discriminating between various pathologic conditions.

After image acquisition and registration is complete, a Butterworth Smoothing algorithm may be applied to the kinetic curves to smooth out their line shape and to eliminate noise. The algorithm is based on a Fast Fourier Transformation (FFT), and produces faster results when applied on 2ⁿ points. If the acquired data points are not exactly 2ⁿ, additional points may be added at the beginning and the end of the curve. Points added to the beginning of the curve have the same value as the first point, and points added to the end of the curve are an average of the last 4 points. A Butterworth filter may be applied on the spectrum of this data set of 2ⁿ points. This serves to cut off high frequencies. An inverse FFT, and eliminating the extra points, results in the smoothed curve of the raw data set. In another embodiment, a Cubic Spline Interpolation is employed in order to smooth the DR vs time curves. Given the intensities {Iᵢ: i=−1, 0, 1, 2} of the time points {uᵢ: i=−1, 0, 1, 2} of the sequence, the intensity at point 0≦u<1 can be estimated using a B-spline curve of order four (degree three).

Another embodiment may use a bi-exponential fitting in order to smooth the DR vs time curves and determine the dynamic optical parameters. The data are fitted with a function of the form:

DR=a exp(bt)+c exp(dt)

The four parameters of the fitting function can be determined by using the Levenberg-Marquardt algorithm. The Levenberg-Marquardt (LM) algorithm is an iterative technique that locates the minimum of a multivariate function expressed as the sum of squares of non-linear real-valued functions. LM is similar to a combination of steepest descent and the Gauss-Newton method. When the current solution is far from the correct one, the algorithm behaves like a steepest descent method: slow, but guaranteed to converge. When the current solution is close to the correct solution, it becomes a Gauss-Newton method, rapidly converging to the solution.

In some embodiments, a difference based filter may be employed to reject noisy curves. This filter is indented to reject, for example, curves that were corrupted due to glare from the cervical tissue, or due to movement that was not corrected by registration. The difference between the raw and the smoothed data is calculated as follows:

$${\sum }_{i = 5}^{N} {( {DR}_{i}^{smooth} - {DR}_{i}^{raw} )}^{2}$$

If this difference exceeds an empirically determined threshold then this curve may also be rejected.

The imaging workstation may also employ Curve Tendency Prediction. In most cases, dynamic optical parameters can be computed reliably even though the time duration of the examination procedure is shorter than the optimum duration determined experimentally. This is possible in cases where the line shape of the DR vs. time curve is substantially known and predictable after a first set of measurements. For example, the shape of DR vs. time curves may be substantially predictable and linear after they reach their maximum value in the time range 1-2 minutes. This experimental evidence may be used to extrapolate the curves of longer time periods although the actual raw data within these periods are missing (for example, if the examination is interrupted due to patient's discomfort). This evidence may be rejected due to excessive noise. When images related to the shape of the curve are captured, the DR vs. time curves may be extrapolated for each pixel of the image. In case the examination is ended after enough images have been captured, but earlier than the predefined duration, the user is able to observe an extrapolation of the DR vs time curves up to the predefined end point. The extrapolation may be displayed with a different color.

The Curve Tendency Prediction algorithm produces a straight line based on the average slope of the points measured after the curve has passed its maximum point (i.e., descending phase). The line is plotted until it reaches either the last point on the time axis or the reference level. In this way, even if the total number of images have not been acquired or rejected, it is possible to extrapolate from the existing images and continue with the diagnostic calculations.

In some embodiments, the curve is calculated and displayed during the evolution of the image acquisition procedure for at least one image point selected automatically. The image point may be the point with parameter values above a cut-off value, indicating the presence of an abnormality. This may allow the user to focus their attention on potentially abnormal tissue areas.

In some embodiments, the captured and stored images may be colour images, colour image RGB channels, spectral images, black and white images or combinations thereof. In other embodiments, the captured and stored images may be the green channel (G) images of the corresponding colour images.

The exemplary imaging workstation may provide quantitative parameters for expressing and mapping the dynamic optical characteristics derived from registered images and processed DR vs. time curves. These parameters may include the slope, time integral, DR maximum value, or time-to-max from the fitted or unfitted DR vs. time curves. If data fitting is employed using, for example, single or multiple exponential fitting polynomial fitting, fitting parameters may the quantitative parameters.

The imaging workstation may provide high, user-independent diagnostic performance through the use of parameter cut-off values, which discriminate normal from pathologic conditions, as well as low from high grade lesions. The parameter cut-off values may be determined experimentally by comparing the parameter values obtained from a certain tissue area with the results obtained from a standard method, and reefing to a tissue sample obtained from the same tissue area. For example, in the case of cervical tissue, where acetic acid solution is used as the diagnostic marker, it has been found (by comparing the DR time integral taken over four minutes with histology) that an optimum cut-off value for discriminating high grade from non high grade cervical neoplasia may lie in the range of about 500-600 (unitless).

The imaging workstation may also map lesions for diagnosis, biopsy sampling, and treatment, based on the display of the spatial distribution of the dynamic optical parameters. The values of the dynamic optical parameters may be represented as pseoudocolors taken from a pseudocolor scale. The spatial distribution of the pseoudocolors may compose a dynamic pseudocolor map image. One method for calculating and segmenting the dynamic pseudocolor map may involves assigning pseoudocolors to the parameter value ranges. A dynamic pseudocolor map may be generated, representing the spatial distribution of the parameter ranges. The dynamic pseudocolor map may be displayed, and may be overlayed and aligned with the last captured image, or onto the real time displayed image of the tissues after the end of the image acquisition procedure The dynamic curve calculation may be displayed for image points of the dynamic pseudocolor map selected though the interfaces. The dynamic pseudocolor map and display size distribution of at least one pseudocolored area may be segmented, and the dynamic pseudocolor map, aligned with reference to the reference image, may be stored. In some embodiments, the pseoudocolors are assigned to areas with parameter values above and below the cut-off values.

In some embodiments, the dynamic pseudocolor map may guide and document biopsy sampling and treatment. Select clusters of the dynamic pseudocolor map may be overlaid onto the real time displayed image of the tissue, and may overlay closed-line markings through the interfaces. A representative dynamic curve and the parameters corresponding to each marking may be calculated and displayed. The dynamic pseudocolor map may be removed though the interfaces. Biopsy sampling or treatment may be performed by inspecting both the tools for biopsy sampling or treatment, and the markings on the display means, using the markings as guidance for aiming the tools towards the selected tissue areas. Image recording may be activated to record the biopsy sampling and treatment procedure

The pseudo-colors may be attributed to each pixel according to parameter values indicating the presence of an abnormality, such as a disease, when compared to certain cut-off values. If there are pixels for which the dynamic parameter values indicate possible pathologic conditions, the map is segmented in various grades and clusters of pixels of a certain lesion grade may be determined. In some embodiments, the cluster with the higher grade and with a size greater than a certain limit may be automatically located, and a circle may be centered on the pixel corresponding to the gravity center of the lesion. This may be displayed and overlaid on the map.

In some embodiments, the biopsy sampling and treatment procedure may be recorded with, for example, still images, a sequence of images, or video.

In some embodiments, some of the above-described features may be activated through the interfaces. In some embodiments, the activation may be performed automatically using motion tracking algorithms of the biopsy sampling/treatment tool.

The exemplary imaging workstation may provide local magnification of the acquired images, thus enabling a detailed examination without losing the overview of the examined area. This may be accomplished by displaying on the display 110, and within a window of pre-defined dimensions and resolution, a part of the magnified image. The rest of the display may contain the full image recorded by the imaging sensor 115, thus allowing for the simultaneous viewing of a specific magnified area, and the entire field of view. The sub area of the image to be magnified may be selected via the user interface.

In some embodiments, the image magnification step also allows the enhancement of the image characteristics by applying different kinds of spectral filtering, color filtering, and contrast or color channel dynamic range control. These may be selected via the user interface.

One method for providing local magnification may involve configuring the imaging sensor 115 to have a first spatial resolution. The imaging optics 112 may be a lens providing a first magnification. The display means may have a given size and a second spatial resolution, and the overview image captured by the sensor may be displayed at a resolution lesser than or equal to the first resolution on the display means. Providing a first magnification, and then a second magnification, may be achieved by displaying and overlaying selected image sub areas at a resolution at least equal with the first resolution.

According to one exemplary embodiment, the first resolution may be at least 1024×768 pixels, the display 110 may be at least 14 inches diagonal size, the second resolution may be at least 640×420 pixels, the first magnification may be in the range of ×6-×15, and the second magnification may be in the range of ×1.5-×2.5.

In some embodiments, the local magnification may apply to a colour image, colour image channels, a spectral image, an enhanced image, or combinations thereof.

In one embodiment, the workstation may provide means for user-friendly dynamic image data parameter and curve storage and retrieval. These means enable the examination and follow-up to be documented in a dedicated data base. Storage, retrieval, post processing, and analysis may be performed thought the user interface. In one embodiment, the database may be accessed through a touchscreen 502. The database may store data in the computer memory and the user interface may allow a user to retrieve and play back data. Such data may include patient personal data, patient referral reason and history, in vitro and in vivo test results, patient management plan information, some or all of the acquired images, the pseudocolor map, markings with corresponding parameter values and dynamic curves, and images for recording and documenting biopsy sampling or treatment.

Storing and retrieving the data in the database may involve updating the patient record with all the data recorded during an examination performed with the imaging workstation. Such data may include the sequence of acquired images, the pseudocolor map 1102, the markings of the sites selected as biopsy points with their parameter values and dynamic curves, and the biopsy sampling imaging record.

Traditional diagnostic methods involving diagnostic markers suffer from several drawbacks mainly related with the fact that the visual assessment of dynamic optical phenomena is less effective, due to the physiological limitations of the human optical system in detecting and recording fast changing phenomena with different kinetics in different tissue sites.

A solution to this problem is provided by a method and device disclosed by Balas C. (2001) IEEE Trans. on Biomedical Engineering, 48:96-104; Balas C J, et al. (1999) SPIE 3568: 31-37; and PCT Publication No. WO 01/72214 A1, wherein quantitative assessment and mapping of the dynamic optical phenomena generated from the biomarker-tissue interaction is provided. The contents of these publications are incorporated herein by reference.

As indicated above, the present imaging workstation provides improved methods for utilizing diagnostic markers, as compared to previous methods. For example, the present imaging workstation provides a systematic parametric analysis of DOC and comparative evaluation of the derived DOPs in terms of both predictive value and efficiency in discriminating various normal and pathologic conditions.

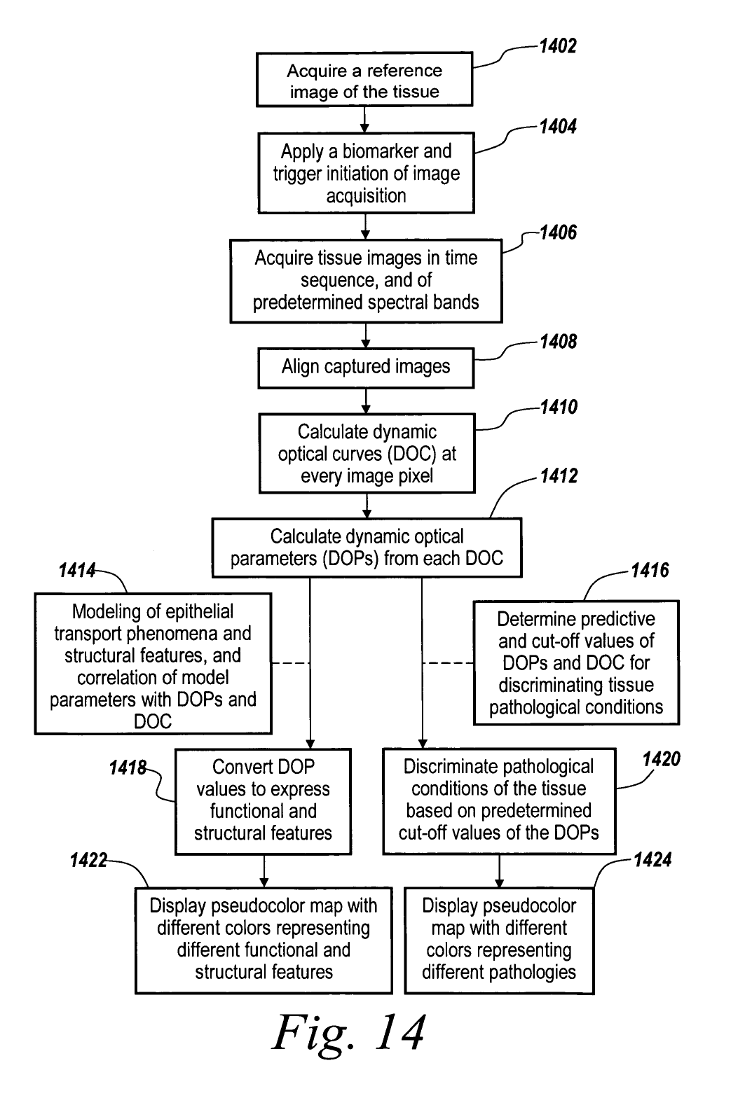

FIG. 14 depicts a flow chart illustrating the basic steps in a method suitable for practicing the teachings discussed herein. At step 1402, the reference image of the tissue before diagnostic marker application is acquired. This step 1402 allows the original optical properties of the examined tissue to be recorded. At step 1404, the diagnostic marker is applied, for instance, by means of an applicator. The diagnostic marker applicator may also provide a triggering signal to initiate image acquisition. In one embodiment, the diagnostic marker provides such a trigger signal less than 1 second after the diagnostic marker application, thus synchronizing and standardizing the acquisition process.

At step 1406, a series of images is acquired in time succession. In one embodiment, the series of images are acquired at a sampling or acquisition rate of between about five and seven seconds, at predetermined spectral bands, and for a predetermined time period of about four minutes. The time period may be determined by taking into account the duration of the optical phenomena induced by the diagnostic marker. Those skilled in the art will recognize that the time period may be less than four minutes, or may extend beyond four minutes (for example, to one or two hours) or any time interval therebetween. Factors such as patient comfort, patient convenience, effectiveness of optical phenomena induced by the diagnostic marker beyond a certain period, system capabilities such as storage capacity and processing capacity, and other like factors may also be used to determine a desired time period. The time period may also be measured in terms of the number of images acquired, for example, thirty images, thirty-five images, forty images and the like. Spectral bands may be selected such that maximum contrast between diagnostic marker responsive and non responsive areas is achieved.

At step 1408, the captured images may be aligned. This step may be desirable for obtaining the temporal variation of light intensity emitted by some or all of the tissue points. Image pixels corresponding to a specific image location may correspond to the same tissue point. In several cases of in vivo measurements, optical sensor-tissue relative movements are present due to any of a number of factors (for example, breathing) as tissue images are successively acquired. Constant relative position between the optical sensor and the examined tissue area may be ensured, for example, through either mechanical stabilization means, and/or image registration algorithms. Proper alignment of the captured images with the reference image acquired at step 1402 allows for the valid extraction of the DOC from an image pixel or group of image pixels corresponding to a specific location of the examined tissue.

At step 1410, the DOC may be calculated from some or all of the acquired series of images. This may be done at every image location (i.e., every pixel location or a location defined by a group of pixels) for selected images. The diffuse reflectance [DR], or fluorescence intensity (FI), may be expressed as a function of time at predetermined spectral bands. The selection of the optical property (DR, FI) may be determined by the property of the employed diagnostic marker to alter either the diffuse reflectance, or fluorescence characteristics, respectively. Proper spectral bands may be selected such that they provide high contrast between diagnostic marker responsive and non-responsive tissues and tissue areas.

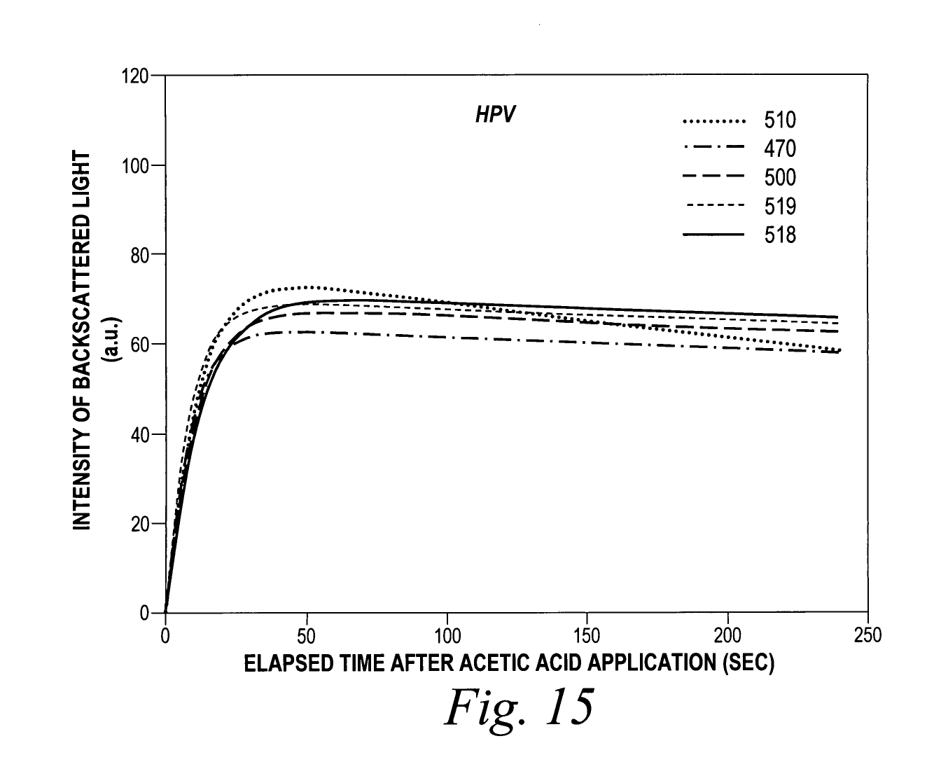

FIGS. 15-18 depict DOC curves obtained from cervical tissue sites interacting with acetic acid solution (the diagnostic marker) corresponding to various pathologies, as classified by histology.

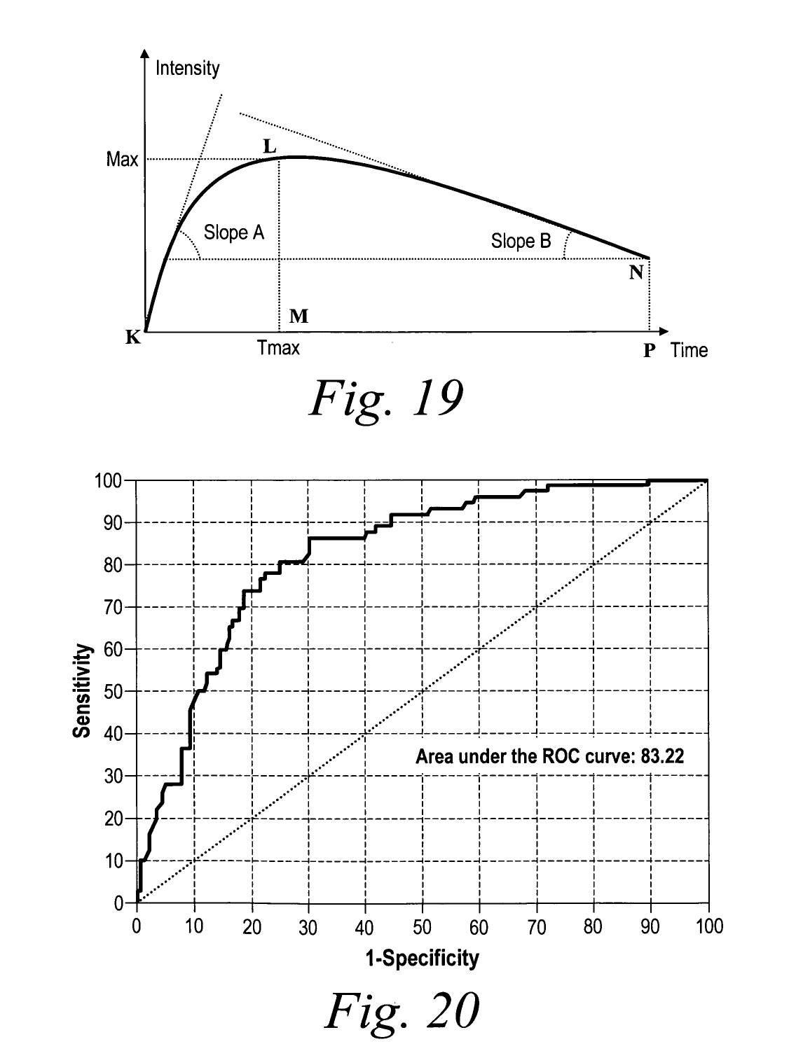

At step 1412, DOPs are calculated from DOCs obtained from image locations (for example, each pixel location or a location defined by a group of pixels) for selected images. A number of parameters expressing the dynamic characteristics of the phenomenon may be derived. Depending on the efficiency of the biomarker in selectively staining tissue abnormalities, DOPs could potentially provide a quantitative means for assessing various tissue pathologies in vivo. These parameters may then be displayed in the form of a pseudocolor map, with different colors representing different parameter values. Such a pseudocolor map may be used to determine the lesion's grade and margins, thus facilitating biopsy sampling, treatment, and lesion management. In one embodiment, a variety of DOPs are calculated from DOCs (e.g., DOC integral over selected time ranges, maxima, slopes as indicated in Table 1 below) expressing the dynamic characteristics of the optical phenomena generated by biomarker-tissue interaction. Detailed analysis of illustrative DOPs is provided below with reference to FIG. 19, where the tissue is cervical epithelium and the biomarker is an acetic acid solution.

At step 1416, the predictive value of the DOPs and DOCs are determined experimentally in a statistically sufficient tissue population by comparing DOP and DOC values. Standard methods providing definite diagnosis may be used, such as histology (gold standards). For those DOPs displaying adequate predictive values, cut-off values that best discriminate various pathological conditions may be determined. For a specific diagnostic marker and epithelial tissue this step could be performed separately and not as a part of the routine implementation of the method. This step may be desirable for correlating DOPs and DOCs with specific pathological conditions.

At step 1420, after establishing the correlation, pathological conditions are discriminated based on predetermined cut-off values of DOPs. Detailed analysis of the assessment of the predictive values of various DOPs in the case where the tissue is cervical epithelium and the diagnostic marker is acetic acid solution is provided below with reference to FIGS. 20-22.

At step 1424, DOP and DOC values representing different pathological conditions and grades may be displayed in the form of a pseudocolor map, wherein different colors represent different grades. The pseudocolor map expresses a pathology map which can be used for the in vivo grading of the lesion, and determining lesion margins, facilitating biopsy sampling, and treating and managing the lesion.

At step 1414, biophysical models of both transport phenomena and structural features of an epithelial tissue may be developed based on the understanding and analysis of diagnostic marker-tissue interaction through in vivo and in vitro experiments. In cases where epithelial transport phenomena are determined by the functional characteristics of the tissue, and in cases where the functional characteristics are expressed in DOPs and DOCs, the model parameters are correlated with the latter, thus providing a means for the in vivo assessment of functional and structural characteristics of the tissue.

At step 1418, DOP values may be converted to express functional and/or structural features of the tissue in various normal and pathological conditions. Functional properties can be determined in living tissues, whereas structural features can be determined in-vitro by analyzing tissue samples (biopsies). The methods of the present invention provide a means for assessing both features in vivo, thus, enabling more complete epithelial system characterization or identification. Complete epithelial system characterization/identification is expected to improve diagnostic performance since various pathological conditions are affecting both functional and structural properties of an epithelial tissue.

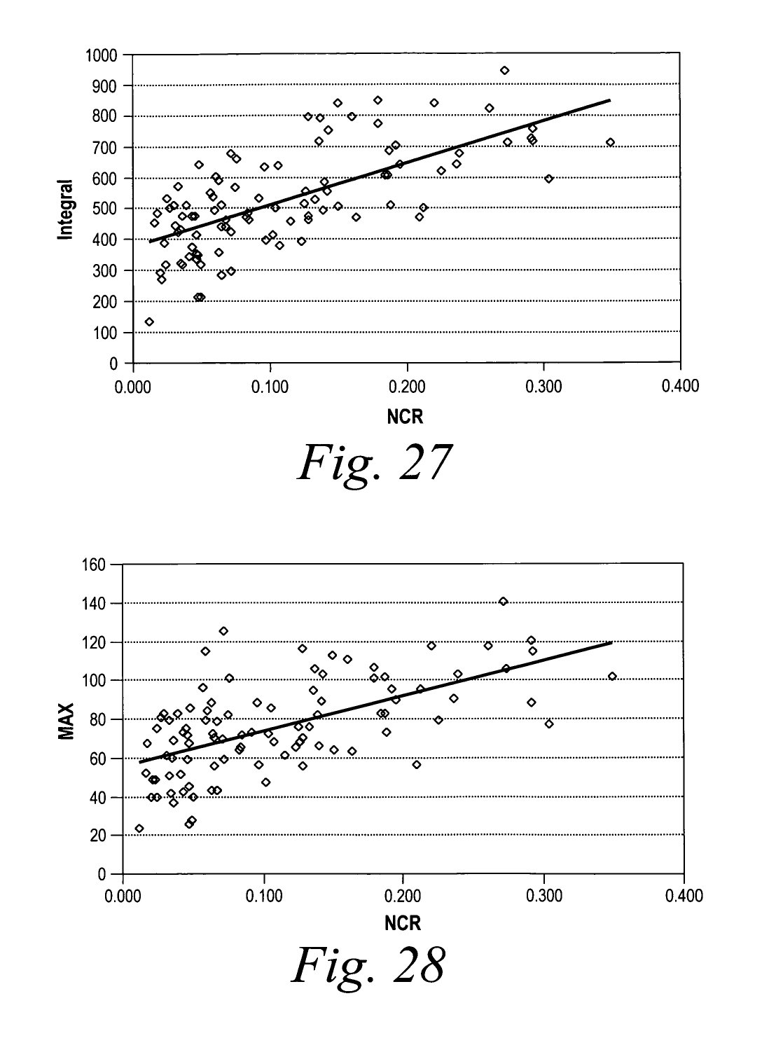

FIGS. 27-28 depict an example: with structural phenomena for the case of cervical cancer where acetic-acid solution is used as a biomarker, DOP values are correlated with quantitative data expressing nuclear density obtained through quantitative pathology methods. The correlation is enables the conversion of DOP to nuclear-to-cytoplasmic-ratio.

At step 1422, a pseudocolor map may be generated with different colors representing different functional and structural features. The pseudocolor map expresses either a tissue functionality and/or structural map, which can be used for the in vivo grading of the lesion, and the determination of the lesion margins, facilitating biopsy sampling, treatment and in general management of the lesion. The pseudocolor map may be also used for in vivo monitoring of the effects of the biomarker in both structural and functional features of the tissue and, consequently, for assessing the efficiency of the biomarker in highlighting abnormal tissue areas.

As an example, in the case of cervical tissue, the appropriate DOPs, and corresponding cut-off values were determined that best discriminate among conditions including normal, HPV (Human Papillomavirus) infection, Inflammation, and Cervical Intraepithelial Neoplasia (CIN) of different grades (see FIGS. 15-18). Acetic acid solution 3-5% was used as the diagnostic marker and the above mentioned measuring procedure for obtaining the DOC was followed. In order to determine the predictive value of DOC and DOPs, experimental data were obtained from a multi-site clinical trial, where 310 women with abnormal Pap-test were enrolled and examined. DOCs were obtained though image capturing in time sequence of the cervical tissue in the blue-green spectral range. The acetic acid responsive tissue areas, as depicted by a DOC and DOPs pseudocolor map, were biopsied and submitted for histological evaluation and grading. The histology classification was then compared with a set of DOPs in order to determine those that best correlate with histology grading through ROC analysis. From the ROC curve, the optimum cut-off values for each parameter, or for a set of parameters, were derived providing the desirable SS and SP values.

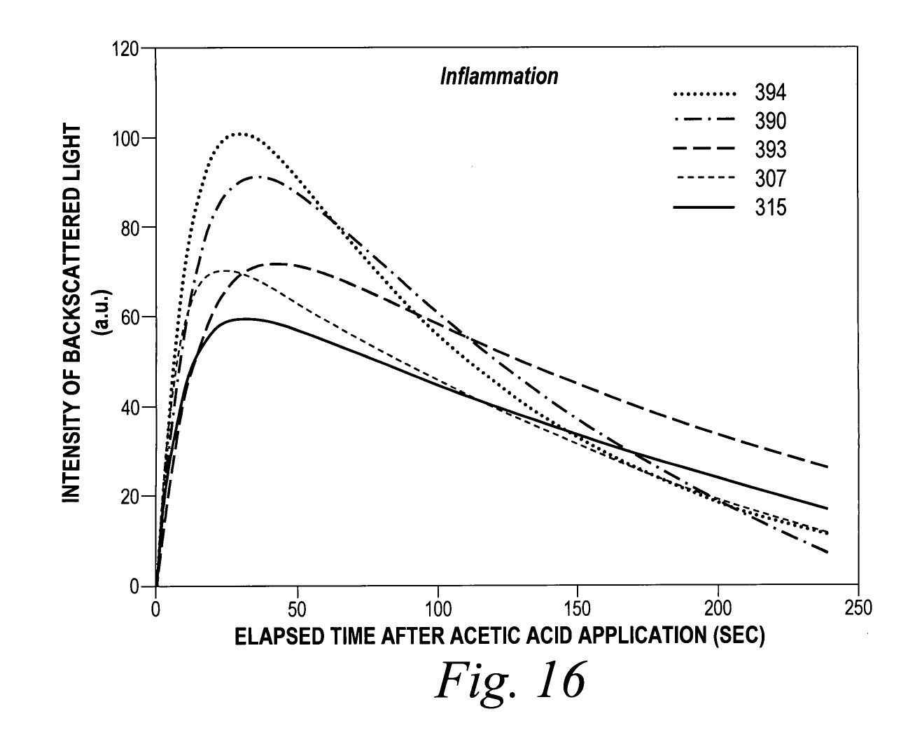

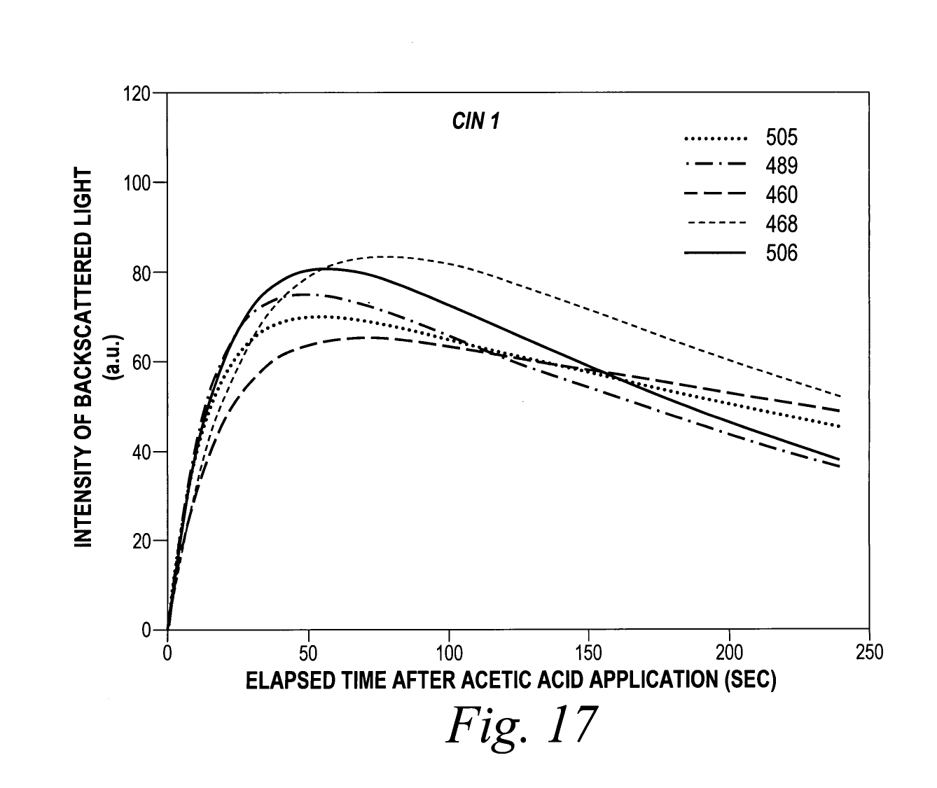

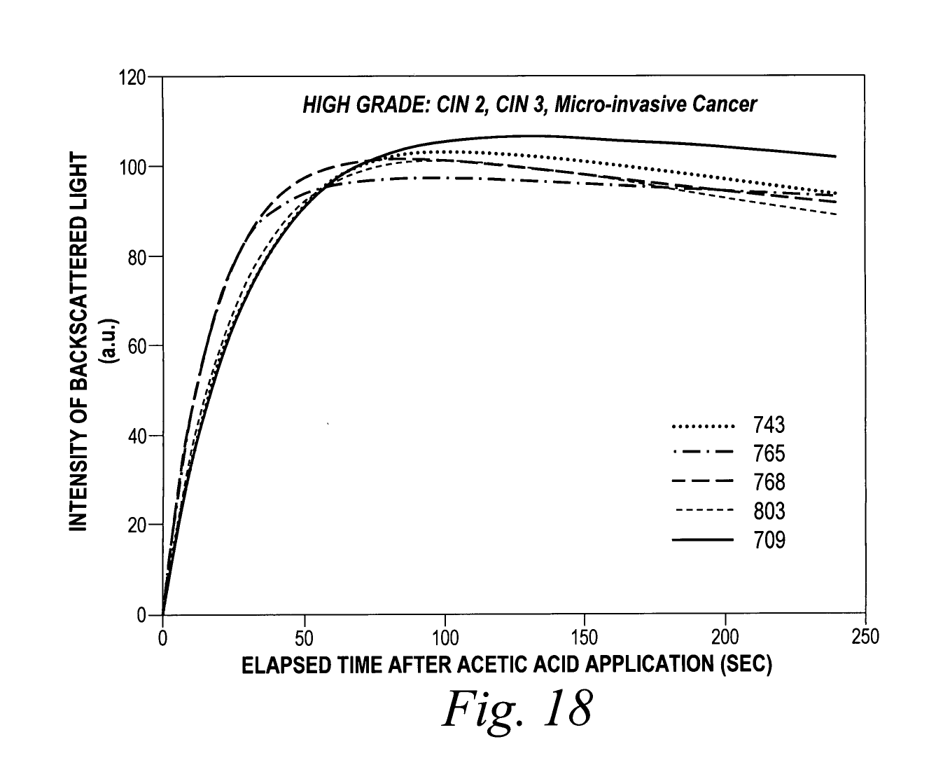

FIGS. 15-18 depict typical DOCs obtained from cervical tissue sites classified by the histologists as: HPV infection (FIG. 15), Inflammation (FIG. 16), CIN1 (FIG. 17), and high-grade (HG) lesions (FIG. 18). As a further categorisation used commonly in clinical practice, HPV, Inflammation, CIN1, or combination thereof, are referred to as low-grade (LG) lesions. HG lesions correspond to either, or combination of, CIN2, CIN3, or Invasive Carcinoma (CA). Histological grades. CIN1, CIN2, and CIN3 are precursors of CA (CIN1-lowest, CIN3-highest). The vertical axes in FIGS. 15-18 corresponds to the IBSL (expressed in arbitrary units), and the horizontal axes represent the elapsed time (in seconds) after the application of acetic acid to the tissue. The DOC corresponding to the various pathologic conditions differ in various ways in terms of intensity-temporal alterations.

In particular, it can be seen that the HPV-classified curves increase almost exponentially and then reach a saturation level, whereas the curves corresponding to inflammation reach a higher peak value earlier, and then decay abruptly. CIN1-classified curves reach their maximum later than the curves corresponding to HPV or inflammation, and then decay with a slow rate, but notably slower than that observed in the inflammation cases. For the HG lesions, the maximum of the curves is reached later and with a higher value than that observed in the HPV and CIN1 cases, whereas the decay rate is very small; much smaller than that seen in the inflammation-classified curves.

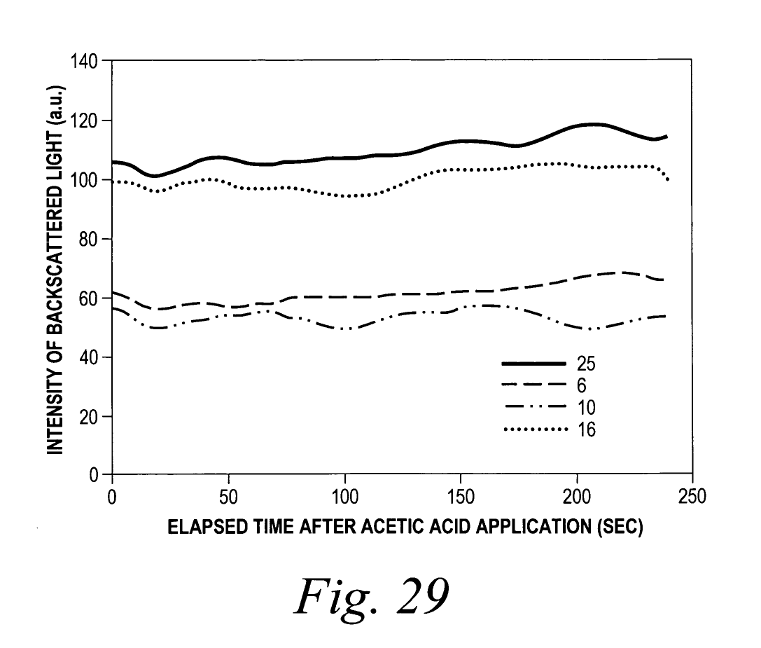

FIG. 29 shows that, in contrast to the above findings, the DOC obtained from a normal tissue site are almost constant across the entire measurement period.

Although helpful, this description of the DOC in relation to a specific pathological condition is rather qualitative. Hence, the following sections describe the quantitative parameters extracted from the dynamic curves which are able to discriminate robustly LG from HG lesions, and HPV infections from HG lesions.

In one embodiment, the DOC obtained from the tissue can be processed using mathematical formulations, including, polynomial, single-, bi-, and multi-exponential fitting, linear and non-linear decomposition, or combinations thereof. This may allow DOPs depicting various characteristics of the recorded DOC in relation to a pathological condition to be derived.

In one embodiment, the derived DOPs can be also weighted based on features particular to the examined tissue sample, such as, for example, patient age, menopausal period (for women), or features characterizing the regional, global, population of the subject whose tissue is examined, or both.

Exemplary DOPs with a high diagnostic value in discriminating LG from HG lesions include:

### 1. Max

This parameter may be defined as the difference between maximum value of the recorded DOC, after the application of a diagnostic marker and DOC value at t=0.

### 2. Integral

This parameter may be defined as the area surrounded by the recorded DOC, and the parallel to the time axis line intersecting the first DOC experimental point. The integral may be calculated for a predetermined time period, which depends on the time duration of optical effects generated by the diagnostic marker-tissue interaction. In the case of cervical tissue and acetic acid solution (the diagnostic marker) the integral may be taken for t=0 to t=4 min. This parameter can be also calculated analytically through the integral of a mathematical formula, after approximation of the measured curve with a closed mathematical form.

### 3. Tmax

This parameter may be defined as the time required for reaching the maximum of the DOC, where the maximum is the Max parameter.

### 4. Area to Max

This parameter may be defined as the area under the curve corresponding to the DOC from t=0 sec (i.e., initialization time of the acetowhitening phenomenon), until t=Tmax. Again, this parameter can also be calculated analytically through the integral of a mathematical formula, after approximation of the measured curve with a closed mathematical form.

### 5. SlopeA

This may be a parameter expressing the rate of intensity increase until the ‘Max’ value. It can be calculated as the first derivative of the curve, or as the average of the intermediate slopes until the ‘Max’ value.

### 6. SlopeB

This may be a parameter expressing the rate of intensity decrease starting from the ‘Max’ value of the curve. It can be calculated as the last derivative of the curve, or as the average of the intermediate slopes, starting from the ‘Max’ value.

FIG. 19 illustrates four of the previously defined parameters on the curve of a DOC: ‘Max’, ‘Tmax’, ‘SlopeA’, and ‘SlopeB’. The other two parameters (‘integral’, and ‘Area to Max’), represent essentially the area enclosed by the indicated points: KLNP, and KLM, respectively.

FIG. 20 illustrates the LG/HG ROC analysis of the cumulative results for the ‘Integral’ parameter described previously. The area under the ROC curve is 0.83, implying high discrimination.

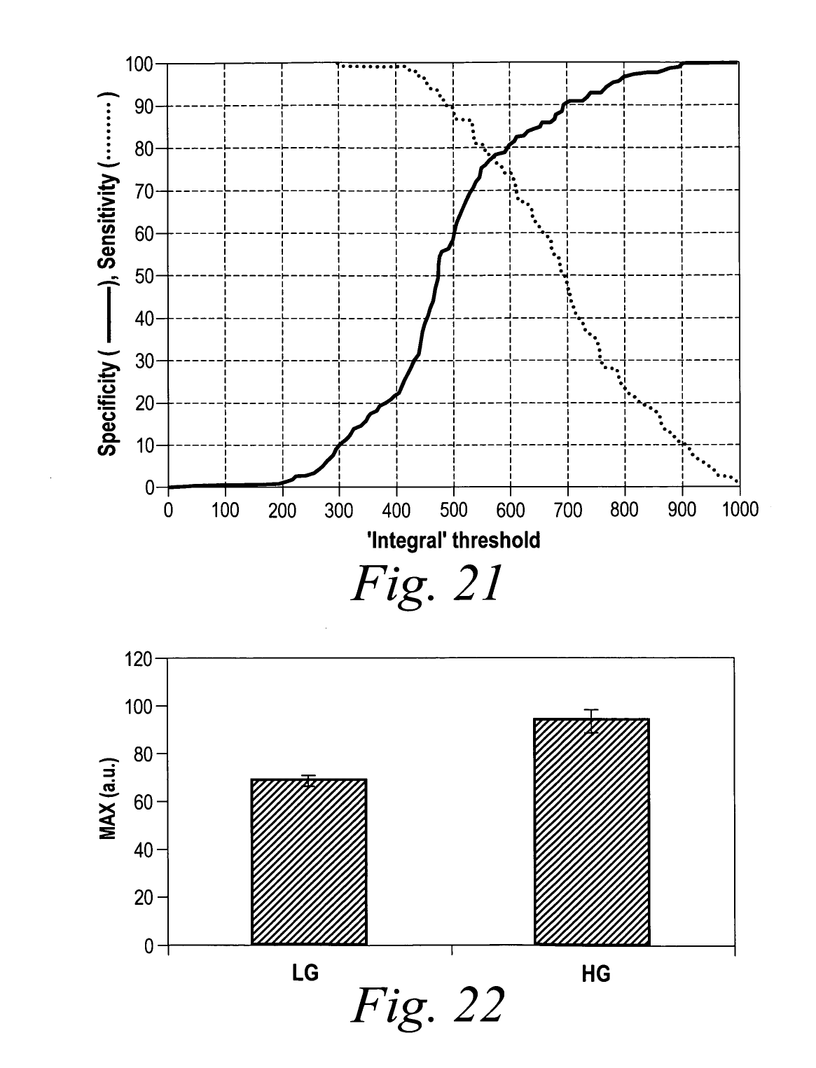

FIG. 21 illustrates the sensitivity (dotted) and specificity (solid) plots derived from the ROC analysis for various values of the ‘Integral’ parameter used for the quantification of the acetowhitening characteristics. It is clearly seen that for a certain value both sensitivity and specificity are maximized reaching 78%.

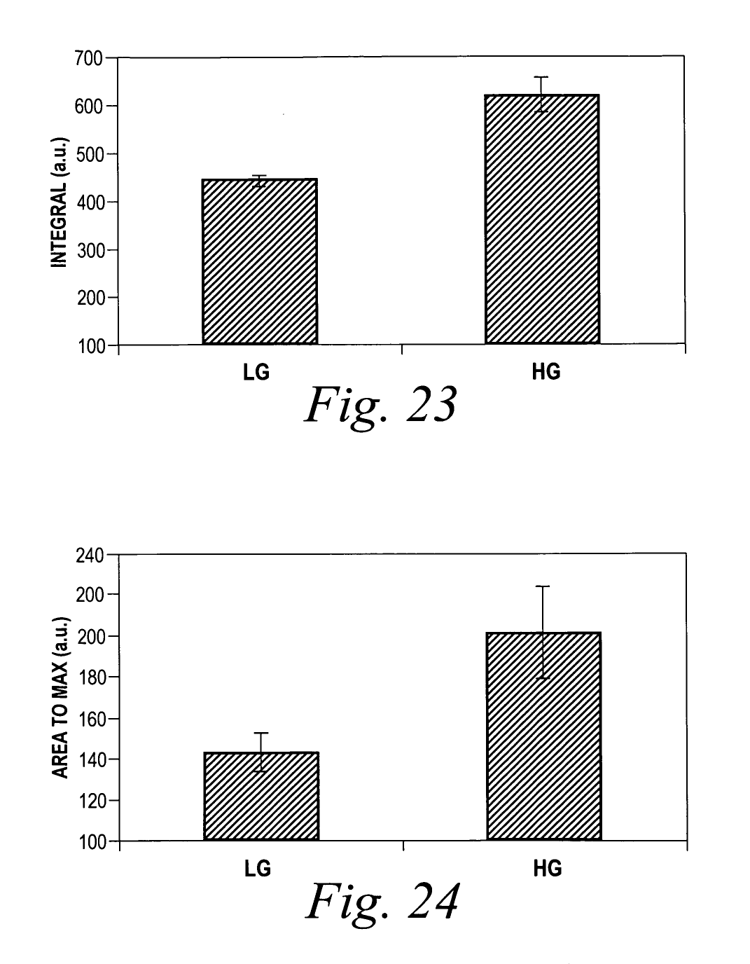

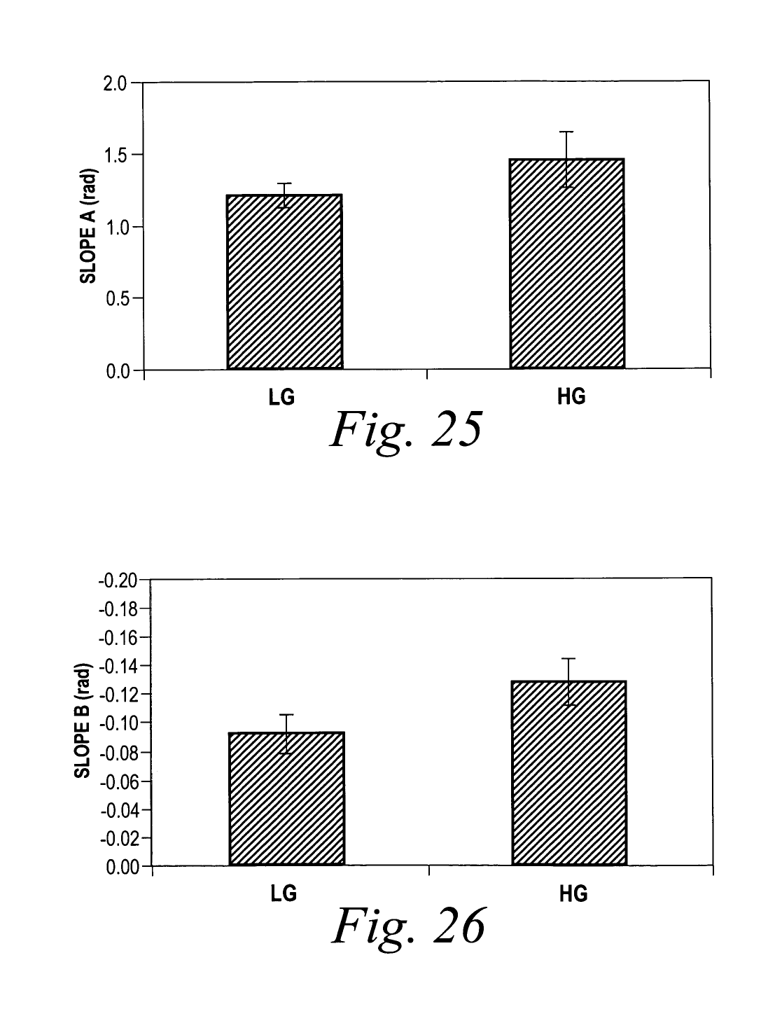

FIGS. 22-26 illustrate the mean values, with corresponding error-bars representing 95% confidence intervals, for some of the parameters described previously, for the LG and HG diagnostic conditions, as concluded through biopsy examination performed by the histologists.

The optimum value ranges in discriminating LG from HG lesions were calculated with ROC analysis, as shown previously for the ‘Integral’ parameter. In particular, for each parameter type the percentage of true positives (TP) and false positives (FP) was calculated for various threshold values spanning the entire range [Pmin, Pmax], where P denotes the value of a specific parameter. The threshold value where the sensitivity (SS=TP), and specificity (SP=100-FP), approximately coincide with one another was used as the optimum (cut-off) value for discriminating LG from HG.

TABLE 1 illustrates the optimum value ranges for discriminating LG from HG lesions for some of the previously defined parameters, leading to a performance dictated by specificity and sensitivity greater than 60%.

TABLE 1

| | Optimum parameter cut-off values for | |
|---|---|---|
| Parameter | LG/HG discrimination | |
| Max | 70 to 90 | (a.u.) |
| Integral* | 480 to 650 | (a.u.) |
| Area To Max | 120 to 170 | (a.u.) |

*The presented integral cut-off values have been calculated from a DOC corresponding to a 4 minute integration time. Different acquisition and integration time periods will result in different cut-off values. The 4 minute time period presented as an example only.

Based on the previous analysis, in one embodiment, the ‘Integral’ parameter of the DOC with the about 480-650 cut-off value range is used for discriminating LG from HG lesions. In one embodiment, the ‘Max’ parameter of the DOC with the about 70-90 cut-off value range is used for discriminating LG from HG lesions. In one embodiment, the ‘Area to Max’ parameter with the about 120-170 cut-off value range is used for discriminating LG from HG lesions. In one embodiment, the ‘SlopeA’ parameter with the about 1.1-1.3 value range is used for discriminating LG from HG lesions. In one embodiment, the ‘SlopeB’ parameter with the about −0.012 to −0.090 cut-off value range is used for discriminating LG from HG lesions.

A similar analysis was also performed for deriving the appropriate cut-off values of the previous parameters for discriminating HPV infections from HG lesions.

TABLE 2 illustrates the optimum value ranges generating specificity and sensitivity greater than 60% for HPV/HG discrimination, for the ‘Max’ and ‘Integral’ parameters.

TABLE 2

| | Optimum parameter cut-off values for |
|---|---|
| Parameter | HPV/HG discrimination |
| Max | 65 to 90 (a.u.) |

In one embodiment, the ‘Integral’ parameter of the DOC with the about 380-490 cut-off value range is used for discriminating HPV infections from HG lesions. In one embodiment the ‘Max’ parameter of the DOC with the about 65-90 cut-off value range is used for discriminating HPV infections from HG lesions.

As shown in FIG. 21, the range of cut-off values provided herein represents the values obtained at different SS and SPs. For example, if the DOP selected were the ‘integral,’ a value of at least 480 could indicate a high-grade cervical neoplasia with a sensitivity of 90% and a specificity of 60% and a value of less than 480 would indicate a low-grade cervical neoplasia with a sensitivity of 90% and a specificity of 60%. Similarly, if the ‘integral’ value selected were a value of 650, then a value of at least 650 would indicate a high-grade cervical neoplasia with a sensitivity of 60% and a specificity of 90% and a value of less than 650 would indicate a low-grade cervical neoplasia with a sensitivity of 60% and a specificity of 90%. Moreover, if the ‘integral’ value selected were a value of 580, then a value of at least 580 would indicate a high-grade cervical neoplasia with a sensitivity of 80% and a specificity of 80% and a value of less than 580 would indicate a low-grade cervical neoplasia with a sensitivity of 80% and a specificity of 80%.

In view of the foregoing, one of skill in the art will appreciate that depending on the SP and SS desired, any cut-off value within the described range may be selected. For example, in the case of the DOP being the ‘integral,’ a value of at least about 480, 490, 500, 510, 520, 530, 540, 550, 560, 570, 580, 590, 600, 610, 620, 630, 640 or 650 could indicate that the cervical tissue being tested is a high grade cervical neoplasia. A value of less than about 480, 490, 500, 510, 520, 530, 540, 550, 560, 570, 580, 590, 600, 610, 620, 630, 640 or 650 in each corresponding case could indicate that the cervical tissue being tested is a low grade cervical neoplasia or a normal tissue.

Similarly, in the case of the DOP being the ‘Max,’ a value of at least about 70, 75, 80, 85, 86, 87, 88, 89 or 90 could indicate that the cervical tissue being tested is a high grade cervical neoplasia. A value of less than about 70, 75, 80, 85, 86, 87, 88, 89 or 90 in each corresponding case could indicate that the cervical tissue being tested is a low grade cervical neoplasia or a normal tissue.

In the case of the DOP being the ‘Area to Max,’ a value of at least about 120, 130, 140, 150, 160 or 170 could indicate that the cervical tissue being tested is a high grade cervical neoplasia. A value of less than about 120, 130, 140, 150, 160 or 170 in each corresponding case could indicate that the cervical tissue being tested is low grade cervical neoplasia or a normal tissue.

In the case of the DOP being the ‘SlopeA,’ a value of at least about 1.1, 1.2 or 1.3 rad. could indicate that the cervical tissue being tested is a high grade cervical neoplasia. A value of less than about 1.1, 1.2 or 1.3 rad. in each corresponding case could indicate that the cervical tissue being tested is low grade cervical neoplasia.

In the case of the DOP being the ‘SlopeB,’ a value of at least about −0.012, −0.020, −0.025, −0.030, −0.040, −0.050, −0.050, −0.060, −0.070, −0.080, or −0.090 could indicate that the cervical tissue being tested is a high grade cervical neoplasia. A value of less than about −0.012, −0.020, −0.025, −0.030, −0.040, −0.050, −0.050, −0.060, −0.070, −0.080, or −0.090 in each corresponding case could indicate that the cervical tissue being tested is low grade cervical neoplasia.

Beyond the ‘hard-clustering’ approach using a cut-off parameter value for discriminating LG from HG lesions, or HPV from HG lesions, more advanced statistical and pattern recognition analysis techniques (such as Bayesian classification, Artificial Neural Networks (ANNs), classification trees) may be employed to extract other linear, or non-linear, parameters for achieving high discrimination, singularly or in combination. In one embodiment, a parametric approach, using Bayesian modelling (as described in, for example, Fukunaga K. (1990) New York: Academic, 2^{nd} Ed.), and a non-parametric approach, using ANNs (Learning Vector Quantization-LVQ, see as described in, for example, Kohonen T., (1986) Int. J. Quant. Chem., Suppl. 13, 209-21), were employed for differentiating the DOPs obtained from corresponding DOC of tissue sites with LG and HG neoplasia. For both Bayes and NN classification, the overall discrimination performance of LG and HG lesions was greater than 75%, for various combinations of the optical parameters described previously, and for a variable number of training sets selected from the overall sample.

In one embodiment, the imaging workstation includes a means for automated cervical screening through the mapping of the dynamic parameter values, and the corresponding cut-off values, showing presence of the disease.

In another embodiment, the imaging workstation includes a means for semi-automated colposcopy through the mapping of the dynamic parameter values and corresponding cut-off values showing presence of the disease. Such a methodology may ensure a base-line colposcopy performance independently of the practitioner's skills, facilitating the overall diagnostic procedure, follow-up, and guidance during biopsy sampling and treatment.

The acetowhitening phenomenon dictated by the dynamic parameters may be interpreted in relation to the functional and structural alterations in the epithelium. In one embodiment, distinctive parameters related to the cervical tissue structural properties are computed and correlated with a number of functional features derived from the DOC recorded from the same tissue sites. Specifically, the nuclear volume and grading of neoplasia (HPV, CIN 1, CIN 2 and CIN3), or cervical cancer are correlated. [Walker D C, et al. (2003) Physiological Measurement, 24:1-15]. The nuclear-to-cytoplasmic-ratio (NCR), which expresses the nuclear density in the epithelial tissue, is a common parameter used to describe this correlation with certain diagnostic conditions. In one embodiment, the cellular structure of the tissue could be assessed by finding the correlation formula between either, or combination, of the aforementioned dynamic parameters with the NCR computed from the biopsy material extracted from corresponding cervical locations. To this end, the NCR may be correlated with the DOC parameters reflecting the abnormal functioning of the epithelium, after acetic acid induction into the tissue area.

In one embodiment, this correlation could lead to the extraction of a pseudocolor map representing the structural properties of the examined cervical tissue at some or all locations, in addition to the map representing the AW kinetic characteristics, along with highlighted sites of high nuclear density. Such an implementation has an exceptional value if, by quantifying the in vivo optical curve obtained from the tissue, which represents an in vivo assessment of the macro-structural tissue state; direct conclusions can be derived about the cellular properties of the tissue, which constitutes a representative view of its structure at a microscopic level.

In order to calculate the NCR for a corresponding number of epithelial tissue sites from which the dynamic parameters were obtained by the method disclosed herein, an equal number of cervical biopsy samples were obtained during colposcopy. The biopsied tissue was processed through standard procedures, immunohistochemically stained, and placed on slides for further evaluation through microscopic image analysis. After acquiring an equivalent number of microscopic histological images, a multistage image-analysis algorithm was employed for segmenting the cell-nuclei displayed in the images [Loukas C G, et al. (2003) Cytometry, 55A(1): 30-42]. The NCR quantity was calculated as the sum of the area occupied by the nuclei enclosed in the epithelium, divided by the overall area of the epithelial tissue. NCR is also known as the ‘cell-packing’ property of the epithelial tissue, expressing essentially the cross-sectional structure of the tissue's cellular population.

FIG. 27 and FIG. 28 depict scatter plots of two different DOPs exhibiting strong correlation coefficients (R), against NCR. These parameters are the ‘Integral,’ and the maximum value (Max), of the dynamic optical curve, as defined previously. The lines in the graphs represent linear regression curves, whereas the DOP to NCR conversion equation and correlation results obtained from least-squares fitting on the experimental data are shown in TABLE 3.

TABLE 3

| NCR vs DOP | Correlation Coefficient | Conversion Equation |
|---|---|---|
| NCR vs‘Integral’ | 0.71 | $NCR = \frac{1}{1349} \times AUC - 0.278$ |
| NCR vs ‘Max’ | 0.64 | $NCR = \frac{1}{181} \times Max - 0.309$ |

From this table it can be seen that both parameters present a significant correlation with the cell-packing property of the tissue. In one embodiment of the method, the linear equations allow conversion of a DOP corresponding to a DOC obtained from a specific tissue site, to the underlying NCR property of the tissue site.

In another embodiment of the method, either of the quantitative pseudocolor maps of ‘Integral’ or ‘Max’ can be converted to the NCR map of the epithelial tissue, using the previously shown conversion formulas.

In addition to the structural alterations of the epithelial tissue in relation to the neoplasia progress, there are also several functional changes in the extracellular and intracellular space of the epithelium after applying the acetic acid solution. In particular, solid tumours are known to live in an acidic microenvironment [Webb S D, at al. (1999) J. Theor. Biol., 196: 237-250; Lee A H, et al. (1998) Cancer Research, 58: 1901-1908; Yamagata M et al. (1996) Br. J Cancer, 73: 1328-1334; and Marion S, et al. (2000) Molecular Medicine Today, 6: 15-19]. Experimental measurements have shown that extracellular pH in tumors is on average 0.5 units lower than that of normal tissues, with tumor extracellular pH lying typically in the range [6.6, 7.0] (see [Yamagata M et al. (1996) Br. J Cancer, 73: 1328-1334]). Tumor cells also have a neutral or slightly alkaline intracellular pH [Marion S, et al. (2000) Molecular Medicine Today, 6: 15-19]. Similar to the normal cells, tumor cells regulate their cytoplasmic pH within a narrow range to provide a favorable environment for various intracellular activities.

Although the issue regarding the presence of acidic extracellular pH in tumors is still controversial, there is a common belief that the acidic environment of tumors arises from the high rate of metabolic acid production, such as lactic acid, and from its inefficient removal from the extracellular space [Webb S D, at al. (1999) J. Theor. Biol., 196: 237-250; Lee A H, et al. (1998) Cancer Research, 58: 1901-1908; Marion S, et al. (2000) Molecular Medicine Today, 6: 15-19; and Prescott D M, et al. (2000) Clinical Cancer Research, 6; (6): 2501-2505]. Tumor cells have a high rate of glycolysis, regardless their oxygen supply level. As a consequence, large quantities of lactic acid (and subsequently H⁺) are produced outwards from the cellular environment. Due to a number of factors such as a disorganized vasculature, or poor lymphatic drainage, and elevated interstitial pressure, the acid clearance (H⁺ clearance) to the blood is very slow, and thus a reversed pH gradient between the extracellular and the intracellular space of tumors cells is observed, [Webb S D, at al. (1999) J. Theor. Biol., 196: 237-250; Lee A H, et al. (1998) Cancer Research, 58: 1901-1908; Yamagata M et al. (1996) Br. J Cancer, 73: 1328-1334; and Marion S, et al. (2000) Molecular Medicine Today, 6: 15-19]. It is also reasonable to assume that the CIN extracellular environment is also acidic (perhaps less acidic), provided that cancer is a transitional process and CIN is a precursor of cancer. Moreover, tumor as well as dysplastic cells are known to employ the same short-term, [Marion S, et al. (2000) Molecular Medicine Today, 6: 15-19], and long-term [Lee A H, et al. (1998) Cancer Research, 58: 1901-1908; Yamagata M et al. (1996) Br. J Cancer, 73: 1328-1334 and Prescott D M, et al. (2000) Clinical Cancer Research, 6; (6): 2501-2505], pH regulation mechanisms as those of normal cells. The excess of protons produced by tumor cell metabolism is excreted from the cell via specific hydrogen pumps [Prescott D M, et al. (2000) Clinical Cancer Research, 6; (6): 2501-2505].

The observation of the acetowhitening effect in the cervix is used in colposcopy to characterize abnormal tissue (i.e. HPV, CIN, or cancer). The acetowhitening effect refers to the phenomenon induced by the application of acetic acid solution to the cervical transformation zone. The acetic acid application selectively induces a transient whitening of abnormal cervical areas. Although it has been used for more than 70 years in clinical practice to locate abnormal areas, the exact physicochemical mechanisms involved in tissue whitening remain still unknown. Similar phenomena are observed when Formic, Propionic, and Butyric, acids are employed as diagnostic markers.

Two major explanations for the interpretation of the acetowhitening effect prevail in the relative literature. In vitro studies have shown that the acetic acid effect is related to the amount of certain cytokeratines (proteins present in epithelial cells) [Maddox P, et al. (1999) Journal of Clinical Pathology, 52: 41-46 and Carrilho C, et al. (2004) Human Pathology, 35: 546-551]. Since in cervical neoplasias the extra-cellular environment is acidic, the topically administered acidic acid molecule is not disassociated to its composing ions and as such can penetrate passively the cell membrane. Entering into the neutral pH cytoplasm the acetic acid molecules are disassociated giving hydrogen and carboxylic ions which interact with nuclear proteins resulting in the alteration of the scattering properties of the abnormal cells selectively.

Cytosolic pH value is crucial for the conformational stability of these proteins. At neutral pH values, proteins are stable in solution. As pH drops, they become unstable and insoluble depending on their pI (isoelectrical point). The process of protein destabilization is called denaturation and this partial denaturation is a reversible process which lasts only for some milliseconds. Denatured or unfolded proteins have a different refractive index, and this may be the reason for the whitening effect. The decrease of pH in normal cells may not be enough to cause the proteins to unfold and perhaps this is the reason that in normal tissue no variation in the IBSL is detected. Thus, the back-scattered light is strongly related to the pH dynamics influenced by the acetic acid penetration in the cervical epithelium. Nevertheless, the proteins that contribute to the effect are not well established. Moreover, each of these proteins may denature at a different pH value.

According to the other interpretation, the action of acetic acid on the epithelium of the transformation zone is related to its concentration [MacLean A B. (2004) Gynecologic Oncology, 95: 691-694]. Acetic acid enters in the cellular environment of the dysplastic layers altering the structure of different nucleoproteins and hence causing the cells to appear opaque. Thus, the dynamics of the back-scattered light follows the dynamics of the acetic acid concentration. In normal tissue, no whitening occurs because the quantity of nucleoprotein is very small.

Based on the above mentioned analysis of the functional and structural features of the epithelium undergoing changes during neoplasia development it is possible to correlate dynamic optical data with epithelial features of diagnostic importance. In particular, the measured dynamic characteristics can be used to decouple various epithelial structural and transport phenomena occurring in time sequence after the application of the diagnostic marker, and to correlate them with in vivo measurable optical parameters thus providing a solution to the inverse problem. In other words, it is possible to obtain information for various epithelial features by measuring in vivo dynamic characteristics and parameters.

In one embodiment of the method, ‘SlopeA’ is used to obtain information for the extracellular acidity, and in turn for the passive diffusion constant, and for the number of cell layers of the stratified epithelium. In another embodiment of the method, ‘Max’ is used to determine the NCR of the epithelium since the intensity of the back-scattered light is proportional to the density of signal sources (cell nuclei). In another embodiment of the method, ‘SlopeB’ is used to obtain information in regard to the cell malfunction in regulating the intracellular pH, and to the existence of disorganized vasculature, or to the poor lymphatic drainage associated with neoplasia development. In another embodiment, the ‘Integral’ parameter is used to obtain combined information for both functional and structural features as described above.

Clinical validation of this biophysical model has been performed by correlating NCR with the ‘Max’ and ‘Integral’ parameters described previously. However, clinical validation of the functional features is clinically impracticable due to the lack of reference methods capable of measuring these features in vivo. In contrast, the method disclosed herein is capable of modelling and predicting in vivo functional features of the tissue, based on its inherent capability of recording, analysing, and displaying dynamic optical characteristics obtained in vivo from a tissue interacting with a biomarker.

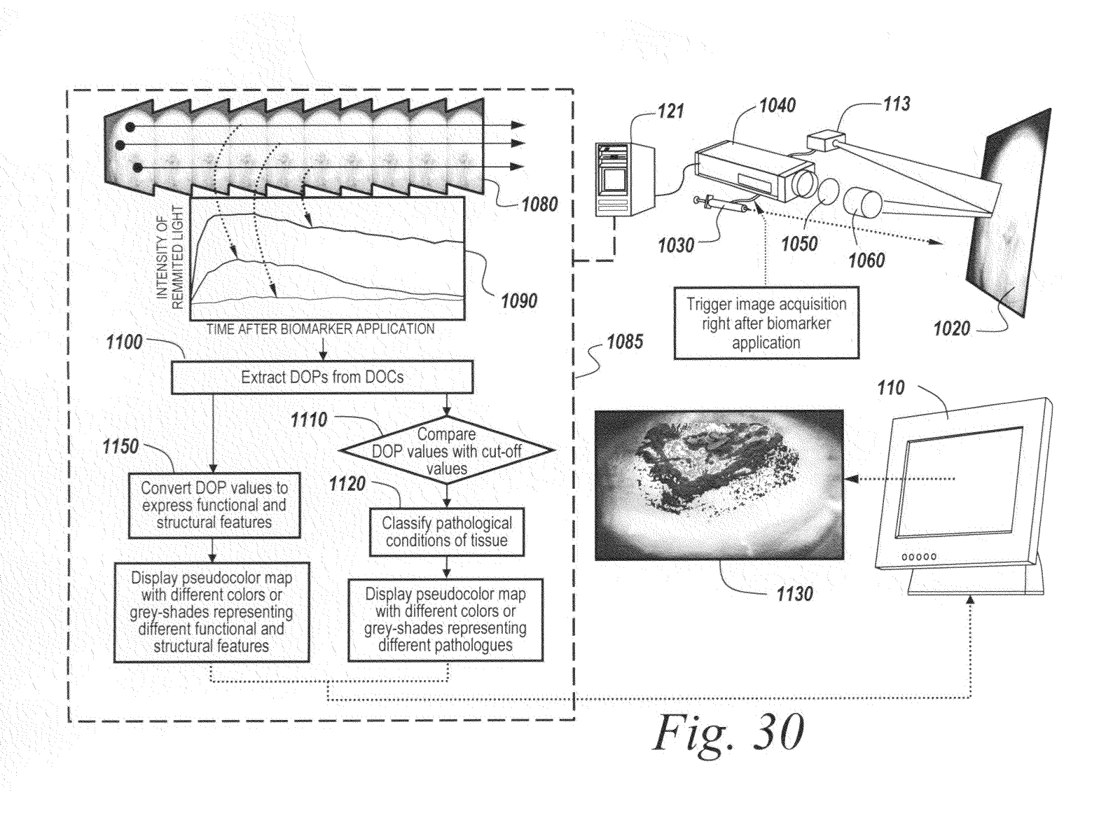

FIG. 30 depicts another illustrative embodiment of the present invention. Computing device 121 may execute instructions embodied on a computer readable medium defining, for example, steps illustrated in an image processing engine 1085 and in conjunction with a hardware set-up utilized to obtain the tissue image data. In particular, the tissue 1020 may be illuminated with a light source 113. After application of a suitable biomarker by means of an applicator 1030, a trigger signal may be provided to initiate image acquisition using an image acquisition device 1040 such as a video CCD or other suitable image acquisition device. Between the tissue 1020 and the image acquisition device 1040 are optical filter 1050 and lenses 1060, for example, one or more zoomable lenses can be interposed. The optical filter 1050 can be tuned to a preferred spectral band, at which a high contrast may be obtained between areas that are subjected to different grade of alterations in their optical reflectance or fluorescence characteristics after administering an appropriate agent.

Before agent administration a tissue image may be obtained as a reference. After agent administration, a series of images 1080, in time succession, at predetermined spectral bands, and for a predetermined time period, may be obtained and stored in memory or a storage device internal to or external to the computing device 121, for further processing by the image processing engine 1085. After proper alignment of some or all of the acquired images, a DOC 1090 may be generated for a specific image location corresponding to the same tissue point. In step 1100, a number of dynamic optical parameters expressing the dynamic characteristics of the phenomenon may be derived from the DOCs.

After extracting the DOPs, in step 1110 their values can be compared with predetermined cut-off values to, in turn, in step 1120, classify various pathological conditions of the tissue. A pseudocolor map 1130, can then be displayed on a display device 110, with different colors, or grey-shades, representing different pathologies. Alternatively, the classification of the various pathological conditions of the tissue can be stored for display at another time or sent to another computing device by, for example, a packet or other unit suitable for use in transporting data in a network environment.

Alternatively, in step 1150, the DOP values can be converted using predetermined mathematical formulas, to express functional and structural features of the tissue. In this case, a pseudocolor map 1130, can be displayed on the display device 110 with different colors, or grey-shades, representing different functional and structural features.

Dynamic Spectral Imaging measures objectively the changes induced by acetic acid and produces a pseudo-colour map of the cervix charting the changes induced by acetic acid. The DySIS instrument can include components depicted in FIG. 30 and/or may include components of the imaging head module 111 and the computer 121 means. The DySiS instrument may be incorporated into the workstation described herein.

The DySIS records these changes using a superior optical and digital camera system. We have studied prospectively 447 women referred to colposcopy in two London clinics and a clinic in Athens using the first clinical prototype. All the women were examined with the DySIS machine and with colposcopy by an operator blinded to the DySIS results. 72 women had high grade disease or pre-clinical invasive disease. An analysis was developed, based on the ability of the system to identify these women.

The receiver operator characteristic curve of the per patient DySIS data had an area under the curve of 0.844, indicating good performance. The sensitivity, specificity and diagnostic odds ratio of the referral smear, colposcopy and DySIS are shown in Table 4.

TABLE 4

| | Referral Smear | Colposcopy | DySIS |
|---|---|---|---|
| Sensitivity | 53% | 49% | 79% |
| Specificity | 86% | 89% | 76% |
| Diagnostic Odds | 6.88 | 7.91 | 11.81 |
| Ratio | | | |

DySIS was more sensitive than colposcopy or the referral smear at the cost of a small reduction in specificity. The improvement in overall performance is illustrated by the diagnostic odds ratio. These results are obtained by an objective process rather than being dependent on the subjective impression of an experienced colposcopist. This instrument would be equally suitable for use by colposcopists, trained nurse practitioners or paramedical staff. It may also have a primary screening role in the developing world.

The contents of all references, figures, patents and published patent applications cited throughout this application are hereby incorporated by reference.

Although the present disclosure is susceptible to various modifications and alternative forms, specific exemplary embodiments are shown in the Figures and will be described herein in detail. It should be understood, however, that the Figures and the Detailed Description are not intended to limit the invention to the particular forms disclosed. On the contrary, the invention covers all modifications, equivalents and alternatives within the spirit and scope of the present invention as defined by the appended claims.

1. (canceled)

2. A supporting structure for an integrated portable imaging workstation operable by an examiner for improving, objectifying and documenting in vivo examination of the uterus, the workstation comprising at least an imaging head module operably-connected to the supporting structure for imaging an examination area of a patient situated on an examination platform,
wherein the supporting structure comprises:
(a) a base member;
(b) a planar positioning structure mounted onto the base member in a manner such that the planar positioning structure can move, relative to the base member, from a position away from the examination area, allowing for the patient's access to the examination platform, to an imaging position, translating at least the imaging head module in close proximity with the examination area;
(c) a space micro-positioning structure disposed directly onto the planar positioning structure;
(d) a weight counterbalancing mechanism integrated in the space micro-positioning structure;
(e) a pivoting structure disposed directly onto the space micro-positioning structure, wherein the imaging head module is disposed directly on the pivoting structure;
(f) wherein motion of the space micro-positioning structure and the pivoting structure may be locked to fix the imaging head module in position in the examination area and unlocked to allow translation away from the examination area; and
(g) a handle for control of a position of the space micro-positioning and the pivoting structures.

3. The supporting structure of claim 2, wherein the planar positioning structure can be locked in the imaging position.

4. The supporting structure of claim 2, wherein the base member comprises rotational members with a defined range of motion and the planar positioning structure is mounted on the rotational members.

5. The supporting structure of claim 4, wherein the rotational members permit an allowable range of motion of about 90°.

6. The supporting structure of claim 2, wherein the planar positioning structure is an articulating extension.

7. The supporting structure of claim 4, wherein the planar positioning structure comprises a vertically supporting foot, fixed near to its other end to that mounted on the rotational members.

8. The supporting structure of claim 2, wherein the planar positioning structure also comprises a lockable, integrated wheel.

9. The supporting structure of claim 2, wherein the base member and the planar positioning structure is a trolley or a collapsible trolley.

10. The supporting structure of claim 3, wherein means for locking the planar positioning structure, the space micro-positioning structure and/or the pivoting structure are selected from among friction elements, mechanical brakes, mechanical stops, hydraulic brakes, pneumatic brakes, electromagnetic brakes, solenoid brakes, and/or electrical motors brakes, positioned properly to control freedom of movement of at least one moving part of at least one of the planar positioning structure, the space micro-positioning and the pivoting structures.

11. The supporting structure of claim 2, wherein the space micro-positioning structure is locked/unlocked using electromagnetic and/or mechanical means.

12. The supporting structure of claim 2, wherein the space micro-positioning structure is an XYZ translator.

13. The supporting structure of claim 12, wherein an XY-plane motion of the XYZ translator is locked/unlocked using electromagnetic means and a Z-axis motion of the XYZ translator is locked/unlocked using a motor coupled with a timing belt and pulley.

14. The supporting structure of claim 2, wherein a motion of the pivoting structure is locked/unlocked using counteracting compression springs and a cam-follower mechanism.

15. The supporting structure of claim 13, wherein the weight counterbalancing mechanism ensures that a suspended weight is balanced using constant force springs mounted fixedly to the Z-axis motion.

16. The supporting structure of claim 2, wherein the pivoting structure is a limited ball joint

17. The supporting structure of claim 16, wherein the handle further incorporates a triggering means to trigger substantially the locking/unlocking of an XY-plane motion and a Z-axis motion of the ball joint.

18. The supporting structure of claim 17, wherein the triggering means comprises a microswitch or a lever with springs acting as a direct brake for the ball joint and as an activator and deactivator of the brakes, placed in remote positions, through at least one of mechanical, hydraulic, pneumatic, electrical transfer of the triggering signal, or combinations thereof.

19. The supporting structure of claim 18, wherein manual force applied to the lever is transmitted to activate the brakes placed in remote positions via a steel wire which is enveloped by a flexible but substantially incompressible tube.

20. The supporting structure of claim 2, wherein the imaging head module is adapted to form a connection with a vaginal speculum located in the examination area and wherein the supporting structure facilitates connection of the imaging head module and the speculum when the imaging head module is in the imaging position to provide an imaging axis and an illumination ray symmetry axis substantially co-linear with a longitudinal axis of the speculum.

21. The supporting structure of claim 2, wherein the workstation further comprises display means for displaying images and/or data of the examination area received from the imaging head module, operably-connected to the supporting structure in a manner such that when the imaging head module is in the imaging position the imaging head module and the display means are located within an examiner's field of vision.

22. The supporting structure of claim 21, wherein the display means is a monitor disposed on a stand, the stand being disposed on the supporting structure, wherein the monitor is placed within the viewing angle of the examiner, which viewing angle also includes the examination area, so that the examiner can observe the examination area, the imaging head module and the monitor without turning his/her head.

23. (canceled)

24. (canceled)

25. The supporting structure of claim 2, wherein the workstation further comprises:
display means for displaying images and/or data of the examination area received from the imaging head module, operably-connected to the supporting structure;
computer means connected to the imaging head module and the display means; and
software means installed in the computer means which causes the computer means to process images obtained by the imaging head module to permit display of an image of the examination area by the display means.

26. The supporting structure of claim 25, wherein the imaging head module comprises:
imaging sensor means coupled with imaging optics means;
light source means for the illumination of a field-of-view of the imaging optics means;
light beam manipulation optics;
diagnostic marker dispensing means, including an application probe;
a speculum with an extension shaft for opening vagina walls; and
a first mechanical support disposed on the pivoting structure, with locking means for detachable connection with the application probe and the shaft of the speculum and a second mechanical support disposed on the pivoting structure, for mounting at least the imaging sensor means and the light source means, wherein the second mechanical support is affixed on the pivoting structure through a linear slider for allowing fine focusing of the imaging sensor means.

27. The supporting structure of claim 26, wherein a first polarizer is placed at a light path of the imaging sensor means and a second polarizer is placed at a light path of the light source means wherein polarization planes of the first and second polarizers are substantially perpendicular to each other.

28. The supporting structure of claim 26, wherein a first imaging sensor is used for imaging the vagina and the cervix of the uterus, and a second imaging sensor is coupled with the imaging optics means for imaging the endocervical canal and the endocervix.

29. The supporting structure of claim 25, wherein two imaging sensors are placed in close proximity and are coupled with at least one lens to achieve stereo-vision of the vagina and of the cervix of the uterus, and wherein the display means provides stereo perception.

30. The supporting structure of claim 26, wherein the diagnostic marker dispensing means is an application mechanism for dispensing a diagnostic marker onto the surface of tissue to be examined, the dispensing means comprising:
the application probe;
a diagnostic marker container; and
means for enabling the application of a diagnostic marker,
wherein the application probe is fixed either directly or indirectly by way of an extension bracket at a certain position on the first mechanical support and wherein an orientation of a longitudinal axis of the application probe is prefixed so that when the imaging head module is connected with the shaft of the speculum, the diagnostic marker is applied substantially homogeneously on to a tissue area of at least equal size with illumination from the light source means and a field-of-view of the imaging sensor.

31. The supporting structure of claim 30, wherein the diagnostic marker container is a dual compartment arrangement comprising a first compartment containing a volume of the diagnostic marker and a second compartment containing a standardized fraction of the volume of the diagnostic marker, pumped from the first compartment through valves and applied through the application probe, with the aid of the means for enabling the application of the diagnostic marker.

32. The supporting structure of claim 30, wherein the means for enabling the application of the diagnostic marker comprises means for enabling manual pumping and application, or means for enabling pumping and application with electronic control.

33. The supporting structure of claim 32, wherein the workstation further comprises at least one sensor for detecting manual pumping and marker application status and for generating an electrical signal for triggering and synchronizing initiation of an image capturing procedure with completion of the application of the diagnostic marker.

34. The supporting structure of claim 32, wherein the means for enabling manual pumping and application comprise a syringe-type mechanism disposed on a structure, enveloping at least in part, a container of the syringe-type mechanism, and wherein the at least one sensor is a pair of electrical contacts disposed at least in part on the enveloping structure, so that manual application moves a piston of the syringe-type mechanism, which in turn brings the electrical contacts in contact at the completion of the application process, generating a triggering signal for initiation and synchronisation of the image capturing procedure.

35. The supporting structure workstation of claim 26, wherein the shaft of the speculum is detachably connectable to the imaging head module with mechanisms chosen from a group including, mechanical locking means, magnetic means, electromagnetic means and pneumatic means.

36. The supporting structure workstation of claim 25, wherein biopsy sampling/treatment procedures are recorded through a video stream together with overlaid digital markings, for documentation purposes and for evaluating biopsy sampling and treatment accuracies.

37. The supporting structure workstation of claim 26, wherein the imaging sensor means has a first spatial resolution, the imaging optics means is a lens providing a constant first magnification and the display means has a given size and a second spatial resolution, wherein an entire image captured by the imaging sensor means is displayed at lesser than or equal to the first spatial resolution on the display means, providing a first magnification, and wherein a second magnification is achieved by displaying and overlaying selected image sub-areas at a resolution at least equal with the first resolution, for allowing magnification of multiple sub-areas without moving the imaging head module and without changing the light beam magnification optics, and for post examination magnification and analysis of the captured images, while maintaining the image overview.

38. The supporting structure workstation of claim 37, wherein the first resolution is at least 1024×768 resolution, and has a data transfer speed of at least 15 f/s, the display size is at least 14 inches diagonal size, the second resolution is at least 640×420, the first magnification is in the range of times 6 to 25 and the second magnification is in the range of times 1.5 to 2.5 which allows for magnification of multiple sub-areas without moving the imaging head module and changing the light beam magnification optics, and for post examination magnification and analysis of the captured images, while maintaining the image overview

39. The supporting structure workstation of claim 25, wherein the workstation further comprises:
means for generating a triggering signal for activating image capturing in a synchronized manner with application of a diagnostic marker; and
a computer readable medium holding computer program instructions,
wherein the computer readable medium holds computer program instructions, causing the workstation to carry out the following actions:
(h) store a reference image in computer memory means of a computer;
(i) capture and store a new reference image replacing the previously stored reference image in the computer memory means;
(j) repeat action (i) until receiving a triggering signal and use the triggering signal for triggering and synchronizing initiation of the image capturing procedure, generated with completion of the application of the diagnostic marker;
(k) store the most recently captured image, just before the arrival of the triggering signal, to be used as a reference image; and/or
(l) initiate the capture, storing and display of images in time sequence, at predetermined time intervals and for a predetermined duration,
(m) align the reference image and the images captured in time-sequence;
(n) calculate and display the remitted light intensity versus time curves;
(o) smooth the defuse reflectance versus time curves using algorithms selected from a group comprising: Butterworth, Fast Fourier Transformation, single and multiple exponential fitting based filters, difference based filters, or combinations thereof;
(p) calculate from the original or fitted/smoothed curves a group of dynamic optical parameters including: time integral, defined as the area under a curve of the remitted light intensity versus time curve calculated for at least a part of the predetermined time duration of the acquisition process; maximum; time-to-max the curve slopes; or combinations thereof;
(q) assign pseoudocolors to the parameter value ranges, to generate the dynamic pseudocolor map representing the spatial distribution of the parameter ranges;
(r) display and overlay the map onto the tissue image; and/or
(s) align the map with at least the reference image for highlighting abnormal areas and for documenting dynamic optical effects through a single image.

40. An integrated portable imaging workstation for improving, objectifying and documenting in vivo examinations of the uterus comprising:
a supporting structure, comprising one or more of:
a base member comprises an eccentric ellipsoid shape, further comprising rotational members with an allowable range of motion of about 90°,
a planar positioning structure comprises an articulating extension mounted onto the rotating members of the base member and wherein the planar positioning structure is a relatively longish member with a vertically supporting foot, fixed near to its other end, with a lockable, integrated wheel, and wherein following the range of motion allowed by the rotating members, the planar positioning structure rotates from its extended (rest) position, allowing for the patient's access to the examination platform, to its closed (imaging) position, translating at least the imaging head module in close proximity with the examination area,
a space micro-positioning structure comprises an XYZ translator disposed directly onto the planar positioning structure,
a weight counterbalancing mechanism is integrated in the space micro-positioning structure and wherein the suspended weight is balanced using constant force springs mounted fixedly to the Z-axis motion element,
a pivoting structure is disposed directly onto the space micro-positioning structure and wherein the pivoting structure comprises a limited ball joint,
XY motion of the XYZ translator is locked/unlocked using electromagnetic means, Z motion of the XYZ translator is locked/unlocked using a motor coupled with a timing belt and pulley, the pivoting structure motion is locked/unlocked using counteracting compression springs and a cam-follower mechanism, and
a handle for the control of the position of the space micro-positioning and pivoting structures is disposed onto the pivoting structure, further incorporating a microswitch to trigger substantially the locking/unlocking of the XY, Z and ball joint motions;
an imaging head module disposed directly onto the pivoting structure, comprising one or more of:
a imaging sensor comprises at least one CCD sensor, coupled with a polarizer with a first orientation of its polarization plane;
a imaging lens comprises a lens with at least 20 mm focal length;
a light source means comprises a white LED light source equipped with optical elements for light beam focusing on an examination area and wherein the light source is coupled with a polarizer with a second orientation of its polarization plane and wherein the second orientation is adjusted to become substantially perpendicular with the first polarization plane;
at least one of the imaging sensor and the illumination means are affixed on the second mechanical support and wherein the second mechanical support is affixed on the pivoting structure through a linear slider for fine focusing;
beam manipulation optics comprises at least one light deflector for deflecting the light rays of at least one of the imaging and illumination means to become substantially co-axial and wherein the light deflector is placed distantly enough from the one of the imaging and illumination means, that is subjected light ray deflection, forming a clear aperture from which the light rays of the other of the imaging and illumination means pass substantially unobstructed;
a diagnostic marker dispenser comprises a bottle containing a volume of the diagnostic marker and is connected via a 2-way valve and tubing to a syringe-like mechanism of fixed volume, and a narrow angle, full-cone, axial spray nozzle, and wherein the nozzle is detachably connected with the extension bracket and aligned properly so that the marker is uniformly applied onto an examination area covering at least the imaging sensor's field-of-view and wherein the nozzle is connected with the syringe-like mechanism via tubes and the valves for transferring to and dispensing from the nozzle the marker, and wherein the syringe-like mechanism is housed in an appropriately designed casing comprising one or more photosensors for detecting the complete depression of the syringe-like mechanism and wherein the output signal of the photosensors is used to synchronize image capturing with application of the diagnostic marker;
a speculum shaft is detachably connectable with the first mechanical support via mechanical locking means disposed onto the first mechanical support via an extension bracket and wherein the locking means is a bayonet type mechanism and wherein the bayonet type mechanism comprises a pre-loaded sleeve with an incorporated angled groove, and a pre-load mechanism for the sleeve, by means of which an extension shaft at the back side of the vaginal speculum is locked into the sleeve, and wherein the pre-loaded sleeve comprises a receptacle for the extension shaft attached to the speculum shaft and wherein the speculum shaft has a dowel pin pressed through it close to its distal end and perpendicular to the axis of the speculum shaft and wherein the dowel pin mates with the receptacle, and wherein the speculum extension shaft comprises shape features to spatially position the speculum longitudinal axis substantially coaxially with the central imaging and illumination axes inside the speculum, when the speculum shaft is locked on the first mechanical support;
computer means disposed directly onto the XY member of the space micro-positioning structure, wherein the computer means is based on multiple core microprocessor which different cores handling different tasks in parallel, and wherein the computer means further includes control means for controlling at least the locking mechanisms and for synchronization and triggering image capturing with agent application, computer memory means, and hardware interface means for connecting computer peripherals including but not limited to: one or more displays, user interface means, a local network, hospital data bases, the internet, and/or printers;
user interface means, wherein the user interface means are selected from among touch-screen, a keyboard, a wireless keyboard, a voice interface, a foot-switch or combinations thereof;
display means, wherein the display means are selected from among, monitors, a touch-screen monitors, head-mounted displays, video goggles and combinations thereof, and wherein the monitor is placed on one side of an examination platform and is disposed directly onto the base member and wherein the monitor is positioned spatially so as to be within the viewing angle (or field of vision) of the user and wherein the viewing angle (or field of vision) also includes the examined area and the imaging head module; and
software means wherein the software is used for programming the computer to perform at least in part one or more of the following functions: image calibration; image capturing initialization; image registration; dynamic curve calculation; processing and analysis; dynamic pseudocolor map calculation and segmentation; biopsy sampling/treatment guiding documentation; image magnification; and/or data base operations for storing, retrieval and post-processing images and data.

41. An integrated portable imaging workstation for improving, objectifying and documenting in vivo examinations of the uterus comprising:
a diagnostic marker dispenser an imaging head module for imaging an examination area comprising:
an imaging sensor, imaging optics and/or a light source;
a means for generating a triggering signal for activating image capturing in a synchronized manner with the application of the diagnostic marker;
computer means connected at least to the imaging head module;
display means connected to the computer means for displaying an image of the examination area;
user interface means; and
a computer readable medium holding computer program instructions
wherein the computer program instructions, cause the workstation perform the following actions:
(a) store a reference image in computer memory means of a computer;
(b) capture and store a new reference image replacing the previously stored reference image in the computer memory means;
(c) repeat this procedure until receiving a triggering signal and use the signal for triggering and synchronization of initiation of the image capturing procedure, generated with completion of the application of the diagnostic marker;
(d) store the most recently captured image, just before the arrival of the triggering signal, to be used as a reference image;
(e) initiate the capture, store and display images in time sequence at predetermined time intervals and at a predetermined time duration;
(f) align the reference image and the images captured in time-sequence;
(g) calculate and display the remitted light intensity versus time curves;
(h) smooth defuse reflectance vs. time curves using algorithms selected from a group comprising: Butterworth, Fast Fourier Transformation, single and multiple exponential fitting based filters, difference based filters or combinations thereof;
(i) calculate from the original or fitted/smoothed curves a group of dynamic optical parameters including: time integral, defined as the area under a curve of the remitted light intensity versus time curve calculated for at least in part of the predetermined time duration of the acquisition process; maximum; time-to-max the curve slopes or combinations thereof;
(j) assign pseoudocolors to the parameter value ranges, to generate a dynamic pseudocolor map representing the spatial distribution of the parameter ranges;
(k) display and overlay the dynamic pseudocolor map onto the tissue image; and
(l) align the dynamic pseudocolor map with at least the reference image for highlighting abnormal areas and for documenting dynamic optical effects through a single image.

42. The workstation of claim 41, wherein the imaging sensor is a color imaging sensor and the images captured and stored are color images and green channel images of the color imaging sensor.

43. The workstation of claim 41, wherein the computer program instructions held on the computer readable medium cause image registration employing a rigid registration algorithm based on a similarity metric selected among Fast Fourier Transform (FFT) and Normalized Mutual Information.

44. The workstation of claims 41, wherein the computer program instructions held on the computer readable medium cause image registration employing a deformable registration algorithm based on thin plate spline transformation combined with robust similarity measures and local motion tracking algorithms.

45. The workstation of claim 41, wherein the computer program instructions held on the computer readable medium cause image registration by applying a first registration algorithm to the result of a second registration algorithm.

46. The workstation of claim 41, wherein the time duration of capture and storing of images in time sequence is selected in the range of 1-4 minutes.

47. The workstation claim 41, wherein the dynamic pseudocolor map is used as a guide for manually annotating, through the user interface means, digital markings, overlaid onto the real-time displayed image and corresponding to image areas indented to be biopsied/treated for guiding biopsy sampling and documentation of the biopsy sampling procedure.

48. The workstation of claim 47, wherein the digital markings are selected automatically through segmentation and analysis of the dynamic pseudocolor map

49. The workstation of claim 47, wherein biopsy sampling/treatment procedures are recorded though a video stream together with the overlaid digital markings for documentation purposes and for evaluating biopsy sampling and treatment accuracies.

50. An integrated portable imaging workstation for improving, objectifying and documenting in vivo examinations of the uterus comprising:
an imaging head module for imaging an examination area, comprising one or more of an imaging sensor, imaging optics and/or a light source;
computer means connected to the imaging head module;
display means connected to the computer means for displaying an image of the examination area;
user interface means, and;
software means installed in the computer means, which causes the computer means to capture, store and process images obtained by the imaging head module to permit display of an image of the examination area by the display means,
wherein the imaging sensor has a first spatial resolution, the imaging optics is a lens providing a constant first magnification, the display means has a given size and a second spatial resolution and wherein the entire image captured by the sensor is displayed at lesser or equal than the first resolution on the display means providing a first magnification, and wherein a second magnification is achieved by displaying and overlaying selected image sub-areas at a resolution at least equal with the first resolution, for allowing magnification of multiple sub-areas, without moving the imaging head and without changing magnification optics, and for post examination magnification and analysis of the captured images, while maintaining the image overview.

51. The workstation of claim 50, wherein the first resolution is at least 1024×768 resolution, and has a data transfer speed at least 15 f/s, the display size is at least 14 inches diagonal size, the second resolution is at least 640×420, the first magnification is in the range of times 6 to 25 and the second magnification is in the range of times 1.5 to 2.5, for allowing magnification of multiple sub-areas, without moving the imaging head and changing magnification optics, for post examination magnification and analysis of the captured images, while maintaining the image overview.

52. The workstation of claim 50, further comprising data base means integrated in the computer memory means allowing for retrieval and play-back through the interface means of a group of data including but not limited to: patient personal data, patient referral reason and history, in vitro and in vivo test results, patient management plan, at least a subset of the acquired images, the pseudocolor map, the markings with the corresponding the parameter values and the dynamic curves, image streams documenting and documenting biopsy sampling/treatment.

53. An in vivo examination workstation for in vivo examination of a uterus, the workstation comprising:
a diagnostic marker dispenser for dispensing a diagnostic marker;
an imaging head module for acquiring an image of an examination area within the uterus, the imaging head module comprising,
an imaging sensor,
imaging optics, and
a light source;
a triggering means for generating a triggering signal for activating image acquisition of the examination area in synchronization with dispensing of the diagnostic marker from the diagnostic marker dispenser;
a computer means connected at least to the imaging head module, the computer means programmed to cause the workstation to,
(a) acquire a reference image,
(b) store the reference image,
(c) in response to the triggering signal, synchronizing the dispensing of the diagnostic marker and the acquiring of a plurality of images of the examination area, store and display the acquired images in time sequential manner at predetermined time intervals and duration,
(d) align the reference image and the images captured in time-sequence,
(e) calculate and display one or more remitted light intensity vs. time curves,
(f) smooth the remitted light intensity vs. time curves using an algorithm selected from a group consisting of: Butterworth, Fast Fourier Transformation, single and multiple exponential fitting based filters, difference based filters or combinations thereof,
(g) calculate from the original or smoothed curves a group of dynamic optical parameters having one or more values, the group including: time integral, defined as area under the curve of the remitted light intensity vs. time curve calculated for at least in part of the predetermined time duration of the acquisition process, maximum, time-to-max curve slopes, or combinations thereof,
(h) assign pseoudocolors to multiple ranges of the parameter values, to generate a dynamic pseudocolor map representing a spatial distribution of the parameter ranges,
(i) display and overlay the dynamic pseudocolor map onto one of the acquired images, and
(j) align the map with at least the reference image for highlighting abnormal areas and for documenting dynamic optical effects through a single image of the tissue,
a display means connected to the computer means for displaying an image of the examination area; and
a user interface.

54. The workstation of claim 53, wherein the dynamic optical parameter is the time integral taken over the time duration of image capturing in time sequence, defined as area under the curve of the remitted light intensity vs. time curve, and wherein a determined value of about 480-650 a.u. or higher indicates high-grade neoplasia.

55. The workstation of claims 53, wherein the dynamic optical parameter is the Max defined as the difference between maximum value of remitted light intensity vs. time curves after the application of the diagnostic marker and the value of remitted light intensity vs. time curves at t=0, both corresponding to the same pixel and wherein a determined value of Max of about 70-90 or higher indicates high-grade neoplasia.
